SEQUENCE LISTING (100 sequences)

SEQ ID NO: 1 - protein, 12 aa, Artificial Sequence
  Synthetic Sequence
LLADTTHHRPWT

SEQ ID NO: 2 - protein, 12 aa, Artificial Sequence
  Synthetic Sequence
VIGESTHHRPWS

SEQ ID NO: 3 - protein, 12 aa, Artificial Sequence
  Synthetic Sequence
LIADSTHHSPWT

SEQ ID NO: 4 - protein, 12 aa, Artificial Sequence
  Synthetic Sequence
ILAESTHHKPWT

SEQ ID NO: 5 - protein, 12 aa, Artificial Sequence
  Synthetic Sequence
ILAETTHHRPWS

SEQ ID NO: 6 - protein, 12 aa, Artificial Sequence
  Synthetic Sequence
IIGESSHHKPFT

SEQ ID NO: 7 - protein, 12 aa, Artificial Sequence
  Synthetic Sequence
GLGDTTHHRPWG

SEQ ID NO: 8 - protein, 12 aa, Artificial Sequence
  Synthetic Sequence
VLGDTTHHKPWT

SEQ ID NO: 9 - protein, 12 aa, Artificial Sequence
  Synthetic Sequence
IVADSTHHRPWT

SEQ ID NO: 10 - protein, 11 aa, Artificial Sequence
  Synthetic Sequence
STADTSHHRPS

SEQ ID NO: 11 - protein, 12 aa, Artificial Sequence
  Synthetic Sequence
TSGGESTHHRPS

SEQ ID NO: 12 - protein, 12 aa, Artificial Sequence
  Synthetic Sequence
TSGGESSHHKPS

SEQ ID NO: 13 - protein, 12 aa, Artificial Sequence
  Synthetic Sequence
TGSGDSSHHRPS

SEQ ID NO: 14 - protein, 13 aa, Artificial Sequence
  Synthetic Sequence
GSSGESTHHKPST

SEQ ID NO: 15 - protein, 12 aa, Artificial Sequence
  Synthetic Sequence
VGADSTHHRPVT

SEQ ID NO: 16 - protein, 12 aa, Artificial Sequence
  Synthetic Sequence
GAADTTHHRPVT

SEQ ID NO: 17 - protein, 12 aa, Artificial Sequence
  Synthetic Sequence
AGADTTHHRPVT

SEQ ID NO: 18 - protein, 12 aa, Artificial Sequence
  Synthetic Sequence
GGADTTHHRPAT

SEQ ID NO: 19 - protein, 12 aa, Artificial Sequence
  Synthetic Sequence
GGADTTHHRPGT

SEQ ID NO: 20 - protein, 24 aa, Artificial Sequence
  Synthetic Sequence
LLADTTHHRPWTVIGESTHHRPWS

SEQ ID NO: 21 - protein, 36 aa, Artificial Sequence
  Synthetic Sequence
LLADTTHHRPWTVIGESTHHRPWSIIGESSHHKPFT

SEQ ID NO: 22 - protein, 60 aa, Artificial Sequence
  Synthetic Sequence
LLADTTHHRPWTVIGESTHHRPWSIIGESSHHKPFTGLGDTTHHRPWGILAESTHHKPWT

SEQ ID NO: 23 - protein, 24 aa, Artificial Sequence
  Synthetic Sequence
LLADTTHHRPWTILAESTHHKPWT

SEQ ID NO: 24 - protein, 60 aa, Artificial Sequence
  Synthetic Sequence
LLADTTHHRPWTILAESTHHKPWTLLADTTHHRPWTILAESTHHKPWTLLADTTHHRPWT

SEQ ID NO: 25 - protein, 24 aa, Artificial Sequence
  Synthetic Sequence
LLADTTHHRPWTGLGDTTHHRPWG

SEQ ID NO: 26 - protein, 36 aa, Artificial Sequence
  Synthetic Sequence
LLADTTHHRPWTGLGDTTHHRPWGLLADTTHHRPWT

SEQ ID NO: 27 - protein, 60 aa, Artificial Sequence
  Synthetic Sequence
LLADTTHHRPWTGLGDTTHHRPWGLLADTTHHRPWTGLGDTTHHRPWGLLADTTHHRPWT

SEQ ID NO: 28 - protein, 84 aa, Artificial Sequence
  Synthetic Sequence
LLADTTHHRPWTGLGDTTHHRPWGLLADTTHHRPWTGLGDTTHHRPWGLLADTTHHRPWTGLGDTTHHRPWGLLADTTHHRPWT

SEQ ID NO: 29 - protein, 60 aa, Artificial Sequence
  Synthetic Sequence
STADTSHHRPSTSGGESTHHRPSTSGGESSHHKPSTGSGDSSHHRPSGSSGESTHHKPST

SEQ ID NO: 30 - protein, 60 aa, Artificial Sequence
  Synthetic Sequence
VGADSTHHRPVTGAADTTHHRPVTAGADTTHHRPVTGGADTTHHRPATGGADTTHHRPGT

SEQ ID NO: 31 - protein, 108 aa, Artificial Sequence
  Synthetic Sequence
STADTSHHRPSLLADTTHHRPWTTSGGESTHHRPSVGADSTHHRPVTTSGGESSHHKPSGAADTTHHRPVTTGSGDSSHHRPSGSSGESTHHKPSTGGADTTHHRPAT

SEQ ID NO: 32 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 33 - protein, 37 aa, Artificial Sequence
  Linker sequence
TGGSGEGGTGASTGGSAGTGGSGGTTSGEAGGSSGAG

SEQ ID NO: 34 - protein, 5 aa, Artificial Sequence
  Linker sequence
GAGTG

SEQ ID NO: 35 - protein, 12 aa, Artificial Sequence
  Consensus sequence
  X (1)..(1): Xaa=V, L, I, G, S, T or A
  X (2)..(2): Xaa=I, L, V, Q, T, S, G or A
  X (3)..(3): Xaa=G, A, V or S
  X (4)..(4): Xaa=E, D, L or G
  X (5)..(5): Xaa=S, T, P T, E or D
  X (6)..(6): Xaa=T or S
  X (7)..(7): Xaa=H, T or S
  X (8)..(8): Xaa=H or T
  X (9)..(9): Xaa=R, S, K, P or H
  X (10)..(10): Xaa=P, S, R or K
  X (11)..(11): Xaa=W, F, S, P, V, A or G
  X (12)..(12): Xaa=S, T G or A
  X (12)..(12): Xaa=is absent or is S, T G, or A
XXXXXXXXXXXX

SEQ ID NO: 36 - protein, 12 aa, Artificial Sequence
  Synthetic Sequence
AAADTTHHRPWT

SEQ ID NO: 37 - protein, 60 aa, Artificial Sequence
  Synthetic Sequence
AAADTTHHRPWTAAADTTHHRPWTAAADTTHHRPWTAAADTTHHRPWTAAADTTHHRPWT

SEQ ID NO: 38 - protein, 60 aa, Artificial Sequence
  Synthetic Sequence
LLADAAHHRPWTLLADAAHHRPWTLLADAAHHRPWTLLADAAHHRPWTLLADAAHHRPWT

SEQ ID NO: 39 - protein, 60 aa, Artificial Sequence
  Synthetic Sequence
LLADTTAARPWTLLADTTAARPWTLLADTTAARPWTLLADTTAARPWTLLADTTAARPWT

SEQ ID NO: 40 - protein, 24 aa, Artificial Sequence
  Synthetic Sequence
LLADTTHHRPWTLLADTTHHRPWT

SEQ ID NO: 41 - protein, 36 aa, Artificial Sequence
  Synthetic Sequence
LLADTTHHRPWTLLADTTHHRPWTLLADTTHHRPWT

SEQ ID NO: 42 - protein, 60 aa, Artificial Sequence
  Synthetic Sequence
LLADTTHHRPWTLLADTTHHRPWTLLADTTHHRPWTLLADTTHHRPWTLLADTTHHRPWT

SEQ ID NO: 43 - protein, 120 aa, Artificial Sequence
  Synthetic Sequence
STSGSTVIGESTHHRPWSLIADSTHHSPWTILAESTHHKPWTILAETTHHRPWSIIGESSHHKPFTGLGDTTHHRPWGVLGDTTHHKPWTIVADSTHHRPWTGQVLPTTTPSSPSTTSGS

SEQ ID NO: 44 - protein, 98 aa, Artificial Sequence
  Synthetic Sequence
LLADTTHHRPWTVIGESTHHRPWSIIGESSHHKPFTGLGDTTHHRPWGILAESTHHKPWTATGGSGEGGTGASTGGSAGTGGSGGTTSGEAGGSSGAG

SEQ ID NO: 45 - protein, 97 aa, Artificial Sequence
  Synthetic Sequence
LLADTTHHRPWTGLGDTTHHRPWGLLADTTHHRPWTGLGDTTHHRPWGLLADTTHHRPWTTGGSGEGGTGASTGGSAGTGGSGGTTSGEAGGSSGAG

SEQ ID NO: 46 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 47 - protein, 211 aa, Homo sapiens [sequence omitted]

SEQ ID NO: 48 - protein, 158 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGSTFEERK

SEQ ID NO: 49 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 50 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 51 - protein, 207 aa, Homo sapiens
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 52 - protein, 159 aa, Homo sapiens
VPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 53 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 54 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 55 - protein, 402 aa, Homo sapiens
MTALPGPLWLLGLALCALGGGGPGLRPPPGCPQRRLGARERRDVQREILAVLGLPGRPRPRAPPAASRLPASAPLFMLDLYHAMAGDDDEDGAPAERRLGRADLVMSFVNMVERDRALGHQEPHWKEFRFDLTQIPAGEAVTAAEFRIYKVPSIHLLNRTLHVSMFQVVQEQSNRESDLFFLDLQTLRAGDEGWLVLDVTAASDCWLLKRHKDLGLRLYVETEDGHSVDPGLAGLLGQRAPRSQQPFVVTFFRASPSPIRTPRAVRPLRRRQPKKSNELPQANRLPGIFDDVHGSHGRQVCRRHELYVSFQDLGWLDWVIAPQGYSAYYCEGECSFPLDSCMNATNHAILQSLVHLMMPDAVPKACCAPTKLSATSVLYYDSSNNVILRKHRNMVVKACGCH

SEQ ID NO: 56 - protein, 399 aa, Homo sapiens
MAARPGLLWLLGLALCVLGGGHLSHPPHVFPQRRLGVREPRDMQREIREVLGLPGRPRSRAPVGAAQQPASAPLFMLDLYRAMTDDSGGGTPQPHLDRADLIMSFVNIVERDRTLGYQEPHWKEFHFDLTQIPAGEAVTAAEFRIYKEPSTHPLNTTLHISMFEVVQEHSNRESDLFFLDLQTLRSGDEGWLVLDITAASDRWLLNHHKDLGLRLYVETEDGHSIDPGLAGLLGRQAPRSRQPFMVGFFRANQSPVRAPRTARPLKKKQLNQINQLPHSNKHLGILDDGHGSHGREVCRRHELYVSFRDLGWLDSVIAPQGYSAYYCAGECIYPLNSCMNSTNHATMQALVHLMKPDIIPKVCCVPTELSAISLLYYDRNNNVILRRERNMVVQACGCH

SEQ ID NO: 57 - protein, 396 aa, Homo sapiens; identical to SEQ ID NO: 32

SEQ ID NO: 58 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 59 - protein, 454 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 60 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 61 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 62 - protein, 429 aa, Homo sapiens
MCPGALWVALPLLSLLAGSLQGKPLQSWGRGSAGGNAHSPLGVPGGGLPEHTFNLKMFLENVKVDFLRSLNLSGVPSQDKTRVEPPQYMIDLYNRYTSDKSTTPASNIVRSFSMEDAISITATEDFPFQKHILLFNISIPRHEQITRAELRLYVSCQNHVDPSHDLKGSVVIYDVLDGTDAWDSATETKTFLVSQDIQDEGWETLEVSSAVKRWVRSDSTKSKNKLEVTVESHRKGCDTLDISVPPGSRNLPFFVVFSNDHSSGTKETRLELREMISHEQESVLKKLSKDGSTEAGESSHEEDTDGHVAAGSTLARRKRSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 63 - protein, 424 aa, Homo sapiens
MGSLVLTLCALFCLAAYLVSGSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRRNAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 64 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 65 - protein, 450 aa, Homo sapiens
MDLSAAAALCLWLLSACRPRDGLEAAAVLRAAGAGPVRSPGGGGGGGGGGRTLAQAAGAAAVPAAAVPRARAARRAAGSGFRNGSVVPHHFMMSLYRSLAGRAPAGAAAVSASGHGRADTITGFTDQATQDESAAETGQSFLFDVSSLNDADEVVGAELRVLRRGSPESGPGSWTSPPLLLLSTCPGAARAPRLLYSRAAEPLVGQRWEAFDVADAMRRHRREPRPPRAFCLLLRAVAGPVPSPLALRRLGFGWPGGGGSAAEERAVLVVSSRTQRKESLFREIRAQARALGAALASEPLPDPGTGTASPRAVIGGRRRRRTALAGTRTAQGSGGGAGRGHGRRGRSRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR

SEQ ID NO: 66 - protein, 455 aa, Homo sapiens
MDTPRVLLSAVFLISFLWDLPGFQQASISSSSSSAELGSTKGMRSRKEGKMQRAPRDSDAGREGQEPQPRPQDEPRAQQPRAQEPPGRGPRVVPHEYMLSIYRTYSIAEKLGINASFFQSSKSANTITSFVDRGLDDLSHTPLRRQKYLFDVSMLSDKEELVGAELRLFRQAPSAPWGPPAGPLHVQLFPCLSPLLLDARTLDPQGAPPAGWEVFDVWQGLRHQPWKQLCLELRAAWGELDAGEAEARARGPQQPPPPDLRSLGFGRRVRPPQERALLVVFTRSQRKNLFAEMREQLGSAEAAGPGAGAEGSWPPPSGAPDARPWLPSPGRRRRRTAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 67 - protein, 392 aa, Homo sapiens
MVLLSILRILFLCELVLFMEHRAQMAEGGQSSIALLAEAPTLPLIEELLEESPGEQPRKPRLLGHSLRYMLELYRRSADSHGHPRENRTIGATMVRLVKPLTNVARPHRGTWHIQILGFPLRPNRGLYQLVRATVVYRHHLQLTRFNLSCHVEPWVQKNPTNHFPSSEGDSSKPSLMSNAWKEMDITQLVQQRFWNNKGHRILRLRFMCQQQKDSGGLELWHGTSSLDIAFLLLYFNDTHKSIRKAKFLPRGMEEFMERESLLRRTRQADGISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 68 - protein, 1301 aa, Homo sapiens
MKIITYFCIWAVAWAIPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAVVQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQGDDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSNSSDSSDSSDSSNSSESSDSSDSSDSDSSDSSDSSNSNSSDSDSSNSSDSSDSSNSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSSDSSDSSDGSDSDSSNRSDSSNSSDSSDSSDSSNSSDSSDSSDSNESSNSSDSSDSSNSSDSDSSDSSNSSDSSDSSNSSDSSESSNSSDNSNSSDSSNSSDSSDSSDSSNSSDSSNSSDSSNSSDSSDSNSSDSSDSSNSSDSSDSSDSSDSSDSSDSSNSSDSSDSSDSSDSSNSSDSSNSSDSSNSSDSSDSSDSSDSSDSSDSSDSSDSSNSSDSSDSSDSSDSSDSSDSSDSSDSSESSDSSDSSNSSDSSDSSDSSDSSDSSDSSDSSDSSDSSNSSDSSDSSDSSDSSDSSNSSDSSDSSESSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD

SEQ ID NO: 69 - protein, 372 aa, Homo sapiens
MPPPQQGPCGHHLLLLLALLLPSLPLTRAPVPPGPAAALLQALGLRDEPQGAPRLRPVPPVMWRLFRRRDPQETRSGSRRTSPGVTLQPCHVEELGVAGNIVRHIPDRGAPTRASEPASAAGHCPEWTVVFDLSAVEPAERPSRARLELRFAAAAAAAPEGGWELSVAQAGQGAGADPGPVLLRQLVPALGPPVRAELLGAAWARNASWPRSLRLALALRPRAPAACARLAEASLLLVTLDPRLCHPLARPRRDAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 70 - protein, 364 aa, Homo sapiens
MLRFLPDLAFSFLLILALGQAVQFQEYVFLQFLGLDKAPSPQKFQPVPYILKKIFQDREAAATTGVSRDLCYVKELGVRGNVLRFLPDQGFFLYPKKISQASSCLQKLLYFNLSAIKEREQLTLAQLGLDLGPNSYYNLGPELELALFLVQEPHVWGQTTPKPGKMFVLRSVPWPQGAVHFNLLDVAKDWNDNPRKNFGLFLEILVKEDRDSGVNFQPEDTCARLRCSLHASLLVVTLNPDQCHPSRKRRAAIPVPKLSCKNLCHRHQLFINFRDLGWHKWIIAPKGFMANYCHGECPFSLTISLNSSNYAFMQALMHAVDPEIPQAVCIPTKLSPISMLYQDNNDNVILRHYEDMVVDECGCG

SEQ ID NO: 71 - protein, 501 aa, Homo sapiens
MRLPKLLTFLLWYLAWLDLEFICTVLGAPDLGQRPQGTRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAAPGGGRAAQLKLSSCPSGRQPAALLDVRSVPGLDGSGWEVFDIWKLFRNFKNSAQLCLELEAWERGRAVDLRGLGFDRAARQVHEKALFLVFGRTKKRDLFFNEIKARSGQDDKTVYEYLFSQRRKRRAPLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 72 - protein, 450 aa, Homo sapiens; identical to SEQ ID NO: 65

SEQ ID NO: 73 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 74 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 75 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 76 - protein, 560 aa, Homo sapiens
MFARMSDLHVLLLMALVGKTACGFSLMSLLESLDPDWTPDQYDYSYEDYNQEENTSSTLTHAENPDWYYTEDQADPCQPNPCEHGGDCLVHGSTFTCSCLAPFSGNKCQKVQNTCKDNPCGRGQCLITQSPPYYRCVCKHPYTGPSCSQVVPVCRPNPCQNGATCSRHKRRSKFTCACPDQFKGKFCEIGSDDCYVGDGYSYRGKMNRTVNQHACLYWNSHLLLQENYNMFMEDAETHGIGEHNFCRNPDADEKPWCFIKVTNDKVKWEYCDVSACSAQDVAYPEESPTEPSTKLPGFDSCGKTEIAERKIKRIYGGFKSTAGKHPWQASLQSSLPLTISMPQGHFCGGALIHPCWVLTAAHCTDIKTRHLKVVLGDQDLKKEEFHEQSFRVEKIFKYSHYNERDEIPHNDIALLKLKPVDGHCALESKYVKTVCLPDGSFPSGSECHISGWGVTETGKGSRQLLDAKVKLIANTLCNSRQLYDHMIDDSMICAGNLQKPGQDTCQGDSGGPLTCEKDGTYYVYGIVSWGLECGKRPGVYTQVTKFLNWIKATIKSESGF

SEQ ID NO: 77 - protein, 418 aa, Homo sapiens
MRSLLLLSAFCLLEAALAAEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL

SEQ ID NO: 78 - protein, 207 aa, Homo sapiens; identical to SEQ ID NO: 51

SEQ ID NO: 79 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 80 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 81 - protein, 281 aa, Homo sapiens [sequence omitted]

SEQ ID NO: 82 - protein, 370 aa, Homo sapiens
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 83 - protein, 360 aa, Homo sapiens
MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 84 - protein, 391 aa, Homo sapiens
MLRPGGAEEAAQLPLRRASAPVPVPSPAAPDGSRASARLGLACLLLLLLLTLPARVDTSWWYIGALGARVICDNIPGLVSRQRQLCQRYPDIMRSVGEGAREWIRECQHQFRHHRWNCTTLDRDHTVFGRVMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 85 - protein, 355 aa, Homo sapiens
MEPHLLGLLLGLLLGGTRVLAGYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 86 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 87 - protein, 351 aa, Homo sapiens
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 88 - protein, 380 aa, Homo sapiens
MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 89 - protein, 359 aa, Homo sapiens
MPSLLLLFTAALLSSWAQLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK

SEQ ID NO: 90 - protein, 365 aa, Homo sapiens
MLPPLPSRLGLLLLLLLCPAHVGGLWWAVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 91 - protein, 349 aa, Homo sapiens
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 92 - protein, 349 aa, Homo sapiens
MHRNFRKWIFYVFLCFGVLYVKLGALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 93 - protein, 386 aa, Homo sapiens
MLCCIQCLCLVSPFPTLTPCQGGPHCLIPIHLCLTFSLFGRSVNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVTMSNPAVLLGIRRIEAKNERVLFRLLKSSLWLQIYADK

SEQ ID NO: 94 - protein, 351 aa, Homo sapiens
MFLSKPSVYICLFTCVLQLSHSWSVNNFLMTGPKAYLIYSSSVAAGAQSGIEECKYQFAWDRWNCPERALQLSSHGGLRSANRETAFVHAISSAGVMYTLTRNCSLGDFDNCGCDDSRNGQLGGQGWLWGGCSDNVGFGEAISKQFVDALETGQDARAAMNLHNNEAGRKAVKGTMKRTCKCHGVSGSCTTQTCWLQLPEFREVGAHLKEKYHAALKVDLLQGAGNSAAGRGAIADTFRSISTRELVHLEDSPDYCLENKTLGLLGTEGRECLRRGRALGRWERRSCRRLCGDCGLAVEERRAETVSSCNCKFHWCCAVRCEQCRRRVTKYFCSRAERPRGGAAHKPGRKP

SEQ ID NO: 95 - protein, 365 aa, Homo sapiens
MLDGSPLARWLAAAFGLTLLLAALRPSAAYFGLTGSEPLTILPLTLEPEAAAQAHYKACDRLKLERKQRRMCRRDPGVAETLVEAVSMSALECQFQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETALKVGSTTNEAAGEAGAISPPRGRASGAGGSDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 96 - protein, 357 aa, Homo sapiens
MRPPPALALAGLCLLALPAAAASYFGLTGREVLTPFPGLGTAAAPAQGGAHLKQCDLLKLSRRQKQLCRREPGLAETLRDAAHLGLLECQFQFRHERWNCSLEGRMGLLKRGFKETAFLYAVSSAALTHTLARACSAGRMERCTCDDSPGLESRQAWQWGVCGDNLKYSTKFLSNFLGSKRGNKDLRARADAHNTHVGIKAVKSGLRTTCKCHGVSGSCAVRTCWKQLSPFRETGQVLKLRYDSAVKVSSATNEALGRLELWAPARQGSLTKGLAPRSGDLVYMEDSPSFCRPSKYSPGTAGRVCSREASCSSLCCGRGYDTQSRLVAFSCHCQVQWCCYVECQQCVQEELVYTCKH

SEQ ID NO: 97 - protein, 417 aa, Homo sapiens
MGSAHPRPWLRLRPQPQPRPALWVLLFFLLLLAAAMPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 98 - protein, 389 aa, Homo sapiens
MLEEPRPRPPPSGLAGLLFLALCSRALSNEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK

SEQ ID NO: 99 - protein, 354 aa, Homo sapiens
MRARPQVCEALLFALALQTGVCYGIKWLALSKTPSALALNQTQHCKQLEGLVSAQVQLCRSNLELMHTVVHAAREVMKACRRAFADMRWNCSSIELAPNYLLDLERGTRESAFVYALSAAAISHAIARACTSGDLPGCSCGPVPGEPPGPGNRWGGCADNLSYGLLMGAKFSDAPMKVKKTGSQANKLMRLHNSEVGRQALRASLEMKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 100 - protein, 364 aa, Homo sapiens
MDRAALLGLARLCALWAALLVLFPYGAQGNWMWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTC

(19) United States
(12) Patent Application Publication
(10) Pub. No.: US 2023/0203115 A1
(43) Pub. Date: Jun. 29, 2023

(54) THERAPEUTIC COMPOSITIONS COMPRISING GRAFT MATERIALS AND BETA-TCP BINDING PEPTIDES AND USES THEREOF

(71) Applicant: Theradaptive, Inc., Frederick, MD (US)

(72) Inventors: Luis ALVAREZ, Lexington, MA (US); Todd HEIL, Mt. Airy, MD (US); David Hunt STEWART, Monrovia, MD (US); Hyeon PARK, Clarksburg, MD (US)

(21) Appl. No.: 17/908,835

(22) PCT Filed: Jan. 5, 2021
(86) PCT No.: PCT/US2021/012219

§ 371 Date: Sep. 1, 2022

Related U.S. Application Data
(60) Provisional application No. 62/986,555, filed on Mar. 6, 2020.

(51) Int. Cl. C07K 14/475; C07K 14/51

(52) U.S. Cl. CPC C07K 14/475; C07K 14/51

(57) ABSTRACT

Provided herein are polypeptides that include one or more β-tricalcium phosphate (βTCP)-binding sequence(s) and uses thereof.

### CROSS-REFERENCE

This application claims the benefit of U.S. Provisional Application No. 62/986,555, filed on Mar. 6, 2020, which is incorporated herein by reference in its entirety.

### STATEMENT AS TO FEDERALLY SPONSORED RESEARCH

This invention was made with government support under W81XWH-18-C-0182 awarded by the Department of Defense. The government has certain rights in the invention.

### SEQUENCE LISTING

The instant application contains a Sequence Listing which has been submitted electronically in ASCII format and is hereby incorporated by reference in its entirety. Said ASCII copy, created on Jan. 5, 2020, is named 50222-704_601_SL.txt and is 211,462 bytes in size.

### TECHNICAL FIELD

The present disclosure relates generally to the field of molecular biology, and more specifically, to methods of treating bone and cartilage loss of defects in a mammal.

### BACKGROUND

In the U.S., an average of six million people break a bone annually. Of these bone defects, the majority heal without problems. However, 5-10% will heal poorly or not at all (American Academy of Orthopaedic Surgeons). Large defects, also known as critical-sized bone defects, may not heal spontaneously and can lead to non-unions. A non-union occurs when there is no indication of healing for at least three months and no expectation of further healing (American Academy of Orthopaedic Surgeons). Currently, bone grafting is regarded as the “gold standard” for treating large or segmental bone defects. However, there are limitations with this treatment such as donor site pain, risk of rejection, limited donor supply and risk of transmission of infectious disease.

The repair of critical and complex bone and cartilage defects is also limited by poor tissue regrowth on implanted orthopedic substrates. Reasons for poor tissue regrowth include the loss of implanted progenitor cells within 48 hours post-implantation and the scarcity of progenitor cells. Another contributing factor is that current orthopedic substrates fail to facilitate sufficient tissue regeneration.

### SUMMARY

The present invention is based on the discovery that the use of composition including β-tricalcium phosphate (β-TCP) and a chimeric polypeptide including at least one β-TCP binding sequence and a mammalian growth factor (e.g., bone morphogenetic protein 2 (BMP-2) can vastly improve bone healing and accelerate tissue regrowth. Thus, provided herein are chimeric polypeptides that include one or more β-TCP binding sequences, and a mammalian growth factor, compositions including any of these chimeric polypeptides (and optionally, β-TCP), and methods of promoting bone or cartilage formulation, methods of replacing and/or repairing bone or cartilage, and methods of treating a bone fraction or bone loss that include administration of any of these compositions. The compositions and methods provided herein can increase and sustain the number of progenitor cells at sites of bone and/or cartilage injury through stem cell capture. The compositions and methods provided herein can also be applied to soft tissue repair or localized delivery of a therapeutic.

Provided herein are compositions including (a) a chimeric polypeptide including (i) one or more β-tricalcium phosphate (β-TCP)-binding sequence(s) and (ii) a mammalian growth factor; and (b) a graft material, where the chimeric polypeptide binds specifically to the graft material.

In some embodiments, the at least one of the one or more β-TCP-binding sequence(s) is selected from the group consisting of: VIGESTHHRPWS (SEQ ID NO: 2), LIADSTHHSPWT (SEQ ID NO: 3), ILAESTHHKPWT (SEQ ID NO: 4), ILAETTHHRPWS (SEQ ID NO: 5), IIGESSHHKPFT (SEQ ID NO: 6), GLGDTTHHRPWG (SEQ ID NO: 7), VLGDTTHHKPWT (SEQ ID NO: 8), IVADSTHHRPWT (SEQ ID NO: 9); STADTSHHRPS (SEQ ID NO: 10), TSGGESTHHRPS (SEQ ID NO: 11), TSGGESSHHKPS (SEQ ID NO: 12), TGSGDSSHHRPS (SEQ ID NO: 13), GSSGESTHHKPST (SEQ ID NO: 14), VGADSTHHRPVT (SEQ ID NO: 15), GAADTTHHRPVT (SEQ ID NO: 16), AGADTTHHRPVT (SEQ ID NO: 17), GGADTTHHRPAT (SEQ ID NO: 18), and GGADTTHHRPGT (SEQ ID NO: 19).

In some embodiments, the chimeric polypeptide includes two or more β-TCP-binding sequences. In some embodiments, the each neighboring pair of the two or more β-TCP binding sequences directly abut each other. In some embodiments, the each neighboring pair of the two or more β-TCP binding sequences are separated by a linker sequence. In some embodiments, the the chimeric polypeptide includes at least two different β-TCP-binding sequences. In some embodiments, the the chimeric polypeptide includes two or more copies of the same β-TCP-binding sequence. In some embodiments, the at least one of the two or more β-TCP-binding sequences is LLADTTHHRPWT (SEQ ID NO: 1) or GQVLPTTTPSSP (SEQ ID NO: 19).

In some embodiments, the the at least two different β-TCP-binding sequences comprise LLADTTHHRPWT (SEQ ID NO: 1) and VIGESTHHRPWS (SEQ ID NO: 2). In some embodiments, the the chimeric polypeptide includes the sequence of LLADTTHHRPWTVIGESTHHRPWS (SEQ ID NO: 20). In some embodiments, the the at least two different β-TCP-binding sequences comprise LLADTTHHRPWT (SEQ ID NO: 1), VIGESTHHRPWS (SEQ ID NO: 2), and IIGESSHHKPFT (SEQ ID NO: 6). In some embodiments, the the chimeric polypeptide includes the sequence of LLADTTHHRPWTVIGESTHHRPWSIIGESSHHKPFT (SEQ ID NO: 21). In some embodiments, the the at least two different β-TCP-binding sequences comprise LLADTTHHRPWT (SEQ ID NO: 1), VIGESTHHRPWS (SEQ ID NO: 2), IIGESSHHKPFT (SEQ ID NO: 6), GLGDTTHHRPWG (SEQ ID NO: 7), and ILAESTHHKPWT (SEQ ID NO: 4). In some embodiments, the the chimeric polypeptide includes the sequence of LLADTTHHRPWTVIGESTHHRPWSIIGESSHHKPFTGLGDTTHHRPWGILAESTHHKPWT (SEQ ID NO: 22). In some embodiments, the the at least two β-TCP-binding sequences comprise LLADTTHHRPWT (SEQ ID NO: 1) and ILAESTHHKPWT (SEQ ID NO: 4). In some embodiments, the the chimeric polypeptide includes at least one copy of the sequence LLADTTHHRPWTILAESTHHKPWT (SEQ ID NO: 23). In some embodiments, the the chimeric polypeptide includes the sequence of LLADTTHHRPWTILAESTHHKPWTLLADTTHHRPWTILAESTHHKPWTLLADTTHH RPWT (SEQ ID NO: 24).

In some embodiments, the at least two β-TCP-binding sequences comprise LLADTTHHRPWT (SEQ ID NO: 1) and GLGDTTHHRPWG (SEQ ID NO: 7). In some embodiments, the the chimeric polypeptide includes at least one copy of the sequence of LLADTTHHRPWTGLGDTTHHRPWG (SEQ ID NO: 25). In some embodiments, the the chimeric polypeptide includes the sequence of LLADTTHHRPWTGLGDTTHHRPWGLLADTTHHRPWT (SEQ ID NO: 26). In some embodiments, the chimeric polypeptide includes the sequence of LLADTTHHRPWTGLGDTTHHRPWGLLADTTHHRPWTGLGDTTHHRPWGLLADTTH HRPWT (SEQ ID NO: 27). In some embodiments, the chimeric polypeptide includes the sequence of LLADTTHHRPWTGLGDTTHHRPWGLLADTTHHRPWTGLGDTTHHRPWGLLADTTH HRPWTGLGDTTHHRPWGLLADTTHHRPWT (SEQ ID NO: 28). In some embodiments, the at least two β-TCP-binding sequences comprise STADTSHHRPS (SEQ ID NO: 10), TSGGESTHHRPS (SEQ ID NO: 11), TSGGESSHHKPS (SEQ ID NO: 12), TGSGDSSHHRPS (SEQ ID NO: 13), and GSSGESTHHKPST (SEQ ID NO: 14).

In some embodiments, the chimeric polypeptide includes the sequence of STADTSHHRPSTSGGESTHHRPSTSGGESSHHKPSTGSGDSSHHRPSGSSGESTHHKPST (SEQ ID NO: 29). In some embodiments, the at least two β-TCP-binding sequences comprise VGADSTHHRPVT (SEQ ID NO: 15), GAADTTHHRPVT SEQ ID NO: 16), AGADTTHHRPVT (SEQ ID NO: 17), GGADTTHHRPAT (SEQ ID NO: 18) and GGADTTHHRPGT (SEQ ID NO: 19). In some embodiments, the chimeric polypeptide includes the sequence of VGADSTHHRPVTGAADTTHHRPVTAGADTTHHRPVTGGADTTHHRPATGGADTTHHRPGT (SEQ ID NO: 30). In some embodiments, the at least two different β-TCP-binding sequences comprise STADTSHHRPS (SEQ ID NO: 10), LLADTTHHRPWT (SEQ ID NO: 1), TSGGESTHHRPS (SEQ ID NO: 11), VGADSTHHRPVT (SEQ ID NO: 15), TSGGESSHHKPS (SEQ ID NO: 12), GAADTTHHRPVT (SEQ ID NO: 16), TGSGDSSHHRPS (SEQ ID NO: 13), GSSGESTHHKPS (SEQ ID NO: 14), and TGGADTTHHRPAT (SEQ ID NO: 18). In some embodiments, the chimeric polypeptide includes the sequence of STADTSHHRPSLLADTTHHRPWTTSGGESTHHRPSVGADSTHHRPVTTSGGESSHHK PSGAADTTHHRPVTTGSGDSSHHRPSGSSGESTHHKPSTGGADTTHHRPAT (SEQ ID NO: 31).

In some embodiments, the mammalian growth factor is selected from the group consisting of: epidermal growth factor (EGF), platelet derived growth factor (PDGF), insulin like growth factor (IGF-1), fibroblast growth factor (FGF), fibroblast growth factor 2 (FGF2), fibroblast growth factor 18 (FGF18), transforming growth factor alpha (TGF-α), transforming growth factor beta (TGF-β), transforming growth factor beta 1 (TGF-β1), transforming growth factor beta 3 (TGF-β3), osteogenic protein 1 (OP-1), osteogenic protein 2 (OP-2), osteogenic protein 3 (OP-3), bone morphogenetic protein 2 (BMP-2), bone morphogenetic protein 3 (BMP-3), bone morphogenetic protein 4 (BMP-4), bone morphogenetic protein 5 (BMP-5), bone morphogenetic protein 6 (BMP-6), bone morphogenetic protein 7 (BMP-7), bone morphogenetic protein (BMP-9), bone morphogenetic protein 10 (BMP-10), bone morphogenetic protein 11 (BMP-11), bone morphogenetic protein 12 (BMP-12) bone morphogenetic protein 13 (BMP-13), bone morphogenetic protein 15 (BMP-15), dentin phosphoprotein (DPP), vegetal related growth factor (Vgr), growth differentiation factor 1 (GDF-1), growth differentiation factor 3 (GDF-3), growth differentiation factor 5 (GDF-5), growth differentiation factor 6 (GDF-6), growth differentiation factor 7 (GDF-7), growth differentiation factor 8 (GDF8), growth differentiation factor 11 (GDF11), growth differentiation factor 15 (GDF15), vascular endothelial growth factor (VEGF), hyaluronic acid binding protein (HABP), collagen binding protein (CBP), fibroblast growth factor 18 (FGF-18), keratinocyte growth factor (KGF), tumor necrosis factor alpha (TNFα), tumor necrosis factor (TNF)- related apoptosis inducing ligand (TRAIL), wnt family member 1 (WNT1), wnt family member 2 (WNT2), wnt family member 2B (WNT2B), wnt family member 3 (WNT3), wnt family member 3A (WNT3A), wnt family member 4 (WNT4), wnt family member 5A (WNT5A), wnt family member 5B (WNT5B), wnt family member 6 (WNT6), wnt family member 7A (WNT7A), wnt family member 7B (WNT7B), wnt family member 8A (WNT8A), wnt family member 8B (WNT8B), wnt family member 9A (WNT9A), wnt family member 9B (WNT9B), wnt family member 10A (WNT10A), wnt family member 10B (WNT10B), wnt family member 11 (WNT11), and wnt family member 16 (WNT16).

In some embodiments, the the mammalian growth factor is BMP-2. In some embodiments, the BMP-2 includes an amino acid sequence of MVAGTRCLLALLLPQVLLGGAAGLVPELGRRKFAAASSGRPSSQPSDEVLSEFELRL LSMFGLKQRPTPSRDAVVPPYMLDLYRRHSGQPGSPAPDHRLERAASRANTVRSFH HEESLEELPETSGKTTRRFFFNLSSIPTEEFITSAELQVFREQMQDALGNNSSFHHRINI YEIIKPATANSKFPVTRLLDTRLVNQNASRWESFDVTPAVMRWTAQGHANHGFVVE VAHLEEKQGVSKRHVRISRSLHQDEHSWSQIRPLLVTFGHDGKGHPLHKREKRQAK HKQRKRLKSSCKRHPLYVDFSDVGWNDWIVAPPGYHAFYCHGECPFPLADHLNSTN HAIVQTLVNSVNSKIPKACCVPTELSAISMLYLDENEKVVLKNYQDMVVEGCGCR (SEQ ID NO: 32). In some embodiments, the BMP-2 consists of the amino acid of SEQ ID NO: 32. In some embodiments, the chimeric polypeptide further includes a linker sequence between a neighboring pair of a β-TCP-binding sequence and the growth factor. In some embodiments, the linker sequence includes TGGSGEGGTGASTGGSAGTGGSGGTTSGEAGGSSGAG (SEQ ID NO: 33). In some embodiments, the linker sequence consists of SEQ ID NO: 33. In some embodiments, the linker sequence includes GAGTG (SEQ ID NO: 34). In some embodiments, the linker sequence consists of SEQ ID NO: 34.

In some embodiments, the chimeric polypeptide binds to β-TCP with a KD of about 1 picomolar to about 100 micromolar. In some embodiments, the graft material is a bone graft material. In some embodiments, the bone graft material is a bone allograft. In some embodiments, the bone graft material is an autologous bone graft. In some embodiments, the bone graft material is a demineralized bone matrix. In some embodiments, the bone graft material is a comminuted bone material. In some embodiments, the comminuted bone graft material includes pulverized bone. In some embodiments, the bone graft material includes at least a portion of a fresh bone, freeze-dried bone, frozen bone, cadaveric bone, or a combination thereof. In some embodiments, the bone graft material is a cortical bone graft material. In some embodiments, the bone graft material is a cancellous bone graft material. In some embodiments, the bone graft material is shaped as a disc, a wafer, a wedge, or a custom shape designed to fill a void in a bone. In some embodiments, the composition is a powder, a putty, a paste, a coating, an aerosol, a scaffold, a substrate, a liquid, or any combinations thereof. In some embodiments, the graft material is a soft tissue graft. In some embodiments, the graft material is a synthetic graft. In some embodiments, the graft material is a hybrid graft including one or more synthetic materials and one or more naturally-derived materials. In some embodiments, the graft material is a xenograft. In some embodiments, the graft material includes hydroxyapatite.

Also provided herein are methods of promoting bone or cartilage formation in a subject in need thereof, the method including administering to the subject a therapeutically effective amount of any of the compositions described herein.

Also provided herein are methods of replacing and/or repairing bone or cartilage in a subject in need thereof, the method including administering to the subject a therapeutically effective amount of any of the compositions described herein.

Also provided herein are methods of treating a bone fracture or bone loss in a subject in need thereof, the method including administering to the subject a therapeutically effective amount of any of the compositions described herein.

In some embodiments, the subject has a bone fracture. In some embodiments, subject has a bone defect. In some embodiments, subject has a cartilage tear or cartilage defect. In some embodiments, subject has cartilage loss.

Also provided herein are kits including any of the composition described herein.

The term “subject” as used herein refers to any mammal. A subject therefore refers to, for example, mice, rats, dogs, cats, horses, cows, pigs, guinea pigs, rats, humans, monkeys, and the like. When the subject is a human, the subject may be referred to herein as a patient. In some embodiments, the subject or “subject in need of treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon), a human, or a rodent (e.g., a mouse, a guinea pig, a hamster, or a rat). In some embodiments, the subject or “subject in need of treatment” may be a non-human mammal, especially mammals that are conventionally used as models for demonstrating therapeutic efficacy in humans (e.g., murine, lapine, porcine, canine, or primate animals) may be employed.

The term “bone defect” refers to the absence or loss (e.g., partial loss) of bone at an anatomical location in a subject where it would otherwise be present in a control healthy subject. A bone defect may be the result of, e.g., an infection (e.g., osteomyelitis), a tumor, a trauma, or an adverse event of a treatment. A bone defect may also affect the muscles, soft tissue, tendons, or joints surrounding the bone defect and cause injury. In some embodiments, a bone defect includes damage to a soft tissue.

The term “cartilage defect” refers to the absence or loss (e.g., partial loss) of cartilage at an anatomical location in a subject where it would otherwise be present in a control healthy subject. A cartilage defect may be the result of, e.g., disease, osteochondritis, osteonecrosis, or trauma. For example, a cartilage defect may affect the knee joint.

“Affinity” refers to the strength of the sum total of non-covalent interactions between a β-TCP binding sequence (or a chimeric polypeptide or polypeptide including a β-TCP binding sequence) and its binding partner (e.g., β-TCP). Affinity can be measured by common methods known in the art, including those described herein. Affinity can be determined, for example, using surface plasmon resonance (SPR) technology (e.g., BIACORE®) or biolayer interferometry (e.g., FORTEBIO®). Additional methods for determining affinity are known in the art.

The term “about,” as defined herein, refers to an amount that is near the stated amount by about 10%, 5%, or 1%, including increments therein. For example, “about” can mean a range including the particular value and ranging from 10% below that particular value and spanning to 10% above that particular value.

The term “allograft,” as defined herein, refers to an allogeneic graft material from a donor (e.g., a cadaver) that is genetically dissimilar to a tissue of a recipient (e.g., a subject receiving the allograft). In some embodiments, the allograft can be a synthetic allograft, a cadaveric allograft, or a combination of both.

The term “autologous graft,” as defined herein, refers to an autologous graft material from a donor (e.g., a subject in need of a graft material) that is genetically similar to a tissue of a recipient (e.g., typically the same subject in need of a graft mateiral).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A are representative in vivo radiographs (lateromedial orientation) of animals from experimental Group A.

FIG. 1B are representative in vivo radiographs (lateromedial orientation) of animals from experimental Group B.

FIG. 1C are representative in vivo radiographs (lateromedial orientation) of animals from experimental Group C.

FIG. 2 is a representative graph of the mean radiographic score (+SEM, n=9-10) of animals from each Group at four weeks- and eight weeks-post-surgery. * P<0.01 vs Group Cl # P<0.01 vs Group V; ┼ P< 0.01 vs Group C at 4 weeks.

FIG. 3A are representative radiographs, sagittal, and transverse micro-CT images at 4-weeks post-operation from animals in Group A, B, or C.

FIG. 3B are representative radiographs, sagittal, and transverse micro-CT images at 4-weeks post-operation from animals in Group A, B, or C.

FIG. 4 is a schematic representation of the varied callus formation in animals from Group A, B, or C at 4 weeks post-surgery. The red-shaded area is an approximation of the mineralized callus volume.

FIG. 5A is a representative graph of micro-CT analyses showing total callus volume in bone specimens obtained at 8 weeks post-surgery from animals in Group A (n=9), B (=n-7), or C (n=8). The data are represented as Mean ± SEM; * P<0.01 vs B, # P<0.0.1 vs C.

FIG. 5B is a representative graph of micro-CT analyses showing bone mineral volume in bone specimens obtained 8 weeks post-surgery from animals in Group A (n=9), B (=n-7), or C (n=8). The data are represented as Mean ± SEM; * P<0.01 vs B, # P<0.0.1 vs C.

FIG. 6A is a representative graph of micro-CT analyses of the diaphyseal region volume of bone speciments obtained 4 weeks post-surgery in animals from Group A (n=10), B (n=8), or C (n=7). Bone mineral volume is expressed as the percentage of bone mineral volume to total volume. The data are represented as Mean ± SEM; * P<0.05 vs B, # P<0.0.5 vs C. A1 vs B1; p=0.059.

FIG. 6B is a representative graph of micro-CT analyses of the cortical bone volume of bone specimens obtained 4 weeks post-surgery in animals from Group A (n=10), B (n=8), or C (n=7). Bone mineral volume is expressed as the percentage of bone mineral volume to total volume. The data are represented as Mean ± SEM; * P<0.05 vs B, # P<0.0.5 vs C.

FIG. 7A is a representative graph of micro-CT analyses of total callus volume in bone specimens obtained 8 weeks post-surgergy from animals in Group A (n=9), B (n=7), or C (n=8). The data are represented as Mean ± SEM; * P<0.01 vs B, # P<0.0.1 vs C.

FIG. 7B is a representative graph of micro-CT analyses of bone mineral volume in bone specimens obtained 8 weeks post-surgery from animals in Group A (n=9), B (n=7), or C (n=8). The data are represented as Mean ± SEM; * P<0.01 vs B, # P<0.0.1 vs C.

FIG. 8 is a representative graph of ultimate load to failure (ULF) at 4- and 8-weeks post-surgery for animals in Group A, B, or C. Data are represented as the mean +/- S.E.M. *P<0.05 vs 4-week groups; #P<0.01 vs 8-week groups.

FIG. 9A are representative histological images of Von Kossa- and H&E-stained bone specimens obtained at 4 weeks post-surgery from animals in Group A, B, or C.

FIG. 9B are representative histological images of TRAP+ osteoclast-stained bone specimens obtained at 4 weeks post-surgery from animals in Groups A, B, or C. White arrows indicate osteoclasts, and yellow arrows indicate osteocytes.

FIG. 10A are representative Von Kossa- and H&E-stained histological images of bone specimens obtained at 8 weeks post-surgery from animals in Group A, B, or C.

FIG. 10B are representative TRAP+ osteoclast-stained histological images of bone specimens obtained at 8 weeks post-surgery from animals in Group A, B, or C. White arrows indicate osteoclasts, and yellow arrows indicate osteocytes.

FIG. 11 is a representative immunoblot of MCF-7 cell lysate probed with an anti-P-Akt antibody, wherein the MCF-7 cells were incubated with β-TCBP-HRG.

FIG. 12 is a representative immunoblot of MCF-7 lysate probed with an anti-P-Akt antibody, wherein the MCF-7 cells were incubated with β-TCBP-HRG (free) or β-TCBP-HRG (β-TCP-bound).

### DETAILED DESCRIPTION

Provided herein are compositions including a chimeric polypeptide further including one or more β-tricalcium phosphate (β-TCP)-binding sequence(s) (e.g., any of the exemplary β-TCP-binding sequences described herein) and a mammalian growth factor (e.g., any of the exemplary mammalian growth factors described herein or known in the art); and a graft material, where the chimeric polypeptide is in contact with the graft material.

In some embodiments, the compositions provided herein include any of the chimeric polypeptides or β-tricalcium phosphate-binding polypeptides described herein. In some embodiments, the compositions can further include a substrate including ϑTCP (e.g., (e.g., a Mastergraft strip, Vitoss Foam Pack, chronOS Strip, Vitoss Micromorsels, LifeInk500, Hyperelastic Bone, bioactive glass, ϑTCP powder, ϑTCP spray dried powder, hydroxyapaptite powder, hydroxyapatite-coated bone screw, ϑTCP granules, hydroxyapatite granules, and ReBOSSIS). In some embodiments, the compositions can further include a substrate including silicon nitride.

Also provided herein are kits that include any of these compositions. Also provided are methods of promoting bone or cartilage formation in a subject in need thereof, methods of replacing and/or repairing bone or cartilage in a subject in need thereof, and methods of treating a bone fraction or bone loss in a subject in need thereof that include administering to the subject any of the compositions described herein.

Non-limiting aspects of these polypeptides, compositions, kits, and methods are described below, and can be used in any combination without limitation. Additional aspects of these polypeptides, compositions, kits, and methods are known in the art.

### Compositions and Kits

Also provided herein are compositions (e.g., pharmaceutical compositions) that include any of the chimeric polypeptides or β-TCP-binding polypeptides described herein. In some embodiments, the compositions can further include a graft material.

In some embodiments, the compositions described herein can have a variety of forms. Examples of such forms include, but are not limited to, a powder, a putty (e.g., moldable form), a putty combined with small cuboid bone chips, a paste, a coating (e.g., a coating on a surface such as a medical device), an aerosol, a scaffold, a substrate, a liquid, a granular form, a porous form, a flowable form, a moldable pack, a malleable strip, morsels, or any combinations thereof. In addition, the provided compositions can be used in a variety of different shapes (e.g., a cross, a ladder, a sphere, an ellipsoid, a square, a triangular pyramid, a rod, a cone, a torus, or a wedge, or any combination thereof) and sizes (e.g., largest average diameter of about 1 mm, about 2 mm, about 3 mm, about 4 mm, about 5 mm, about 6 mm, about 7 mm, about 8 mm, about 9 mm, about 1 cm, about 2 cm, about 3 cm, about 4 cm, about 5 cm, about 6 cm, about 7 cm, about 8 cm, about 9 cm, or about 10 cm). In some embodiments, the compositions described herein can have a customizable shape designed to fit and/or mate with a void in a tissue (e.g., a bone) of a subject.

In some examples, the graft material is a bone graft material. In some embodiments, the graft material includes hydroxyapatite. In some embodiments, the graft material includes synthetic hydroxyapatite, synthetic calcium phosphate materials, tricalcium phosphate, sintered hydroxyapatite, settable hydroxyapatite, or any combination thereof.

In some instances, the bone graft material is a bone allograft (i.e., an allogeneic graft). In some embodiments, the bone allograft includes live cells. In some embodiments, the cells include endogenous bone forming cells (e.g., mesenchymal stem cells, osteogenic progenitor cells, cartilage-forming cells, osteoblasts, bone marrow cells, blood cells, or any combination thereof). In some embodiments, the bone allograft is decellularized. In some embodiments, the bone allograft includes endogenous growth factors. In some examples, the endogenous growth factors include any of the mammalian growth factors disclosed herein. In some examples, the bone allograft includes endogenous vascular endothelial growth factor (VEGF). In some examples, the bone allograft includes endogenous bone morphogenic protein-2 (BMP-2). In some embodiments, the bone allograft is sourced from a living or deceased donor.

In some embodiments, the bone graft material is an autologous bone graft. For example, the autologous bone graft can be sourced from a bone of a patient that does not required treatment (e.g., a healthy bone).

In some examples, the bone graft material is a demineralized bone matrix. In some embodiments, the demineralized bone matrix includes allogeneic or autologous bone material combined with a polymer carrier. In some embodiments, the polymer carrier enhances the handling properties of the demineralized bone matrix. In some embodiments, the polymer carrier is a biodegradable polymer. In some embodiments, the polymer carrier may include proteins, polysaccharides, cellulose, synthetic polymers (e.g., polylactide-co-glycolic acid (PLGA)), surface demineralized bone, mineralized bone, nondemineralized cancellous scaffolds, demineralized cancellous scaffolds, cancellous chips, particulate, demineralized, guanidine extracted, species-specific (allogeneic) bone, specially-treated, particulate, protein extracted, demineralized, xenogenic bone, collagen, synthetic hydroxyapatite, synthetic calcium phosphate materials, tricalcium phosphate, sintered hydroxyapatite, settable hydroxyapatite, polylactide polymers, polyglycolide polymers, polylactide-co-glycolide copolymers, tyrosine polycarbonate, calcium sulfate, collagen sheets, settable calcium phosphate, polymeric cements, settable polyvinyl alcohols, polyurethanes, resorbable polymers, liquid settable polymers, and other biocompatible settable materials. The polymer carrier may further include a polyol (including glycerol or other polyhydroxy compound), a polysaccharide (including starches), a hydrogel (including alginate, chitosan, dextran, pluronics, N,O-carboxymethylchitosan glucosamine (NOCC)), hydrolyzed cellulose, or a polymer (including polyethylene glycol). In some embodiments, the polymer carrier may prevent contact between the demineralized bone matrix and activating agents (e.g., water, enzymes, etc.) and/or degradatory enzymes.

In some embodiments, the bone graft material is a comminuted bone material. In some instances, the comminuted bone graft material includes pulverized bone. Comminuted bone material includes bone material having a particle size that is less than the particle size in an unprocessed bone (e.g., a bone that has not been comminuted). Non-limiting examples of methods and/or techniques to comminute bone material include grinding, pulverizing, milling, blending, sectioning, crushing, triturating, disintegrating, cutting, or any combination thereof. In some embodiments, the bone material is comminuted at a temperature that is less than 4° C. In some embodiments, the bone material is comminuted using a cryogenic tool (e.g., a cryogenic grinder) that reduces the heat generated by the pulverization of bone during the comminuting process.

In some embodiments, the comminuted bone material has an average particle size of about 1 nm to about 50 mm, about 1 nm to about 45 mm, about 1 nm to about 40 mm, about 1 nm to about 35 mm, about 1 nm to about 30 mm, about 1 nm to about 25 mm, about 1 nm to about 20 mm, about 1 nm to about 15 mm, about 1 nm to about 10 mm, about 1 nm to about 5 mm, about 1 nm to about 1 mm, about 1 nm to about 900 Tm, about 1 nm to about 800 Tm, about 1 nm to about 700 Tm, about 1 nm to about 600 Tm, about 1 nm to about 500 Tm, about 1 nm to about 400 Tm, about 1 nm to about 300 Tm, about 1 nm to about 200 Tm, about 1 nm to about 100 Tm, about 1 nm to about 50 Tm, about 1 nm to about 10 Tm, about 1 nm to about 5 Tm, about 1 nm to about 1 Tm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 300 nm, about 1 nm to about 200 nm, about 1 nm to about 100 nm, about 1 nm to about 50 nm, about 1 nm to about 25 nm, about 1 nm to about 10 nm, about 1 nm to about 5 nm, about 5 nm to about 50 mm, about 5 nm to about 45 mm, about 5 nm to about 40 mm, about 5 nm to about 35 mm, about 5 nm to about 30 mm, about 5 nm to about 25 mm, about 5 nm to about 20 mm, about 5 nm to about 15 mm, about 5 nm to about 10 mm, about 5 nm to about 5 mm, about 5 nm to about 1 mm, about 5 nm to about 900 Tm, about 5 nm to about 800 Tm, about 5 nm to about 700 Tm, about 5 nm to about 600 Tm, about 5 nm to about 500 Tm, about 5 nm to about 400 Tm, about 5 nm to about 300 Tm, about 5 nm to about 200 Tm, about 5 nm to about 100 Tm, about 5 nm to about 50 Tm, about 5 nm to about 10 Tm, about 5 nm to about 5 Tm, about 5 nm to about 1 Tm, about 5 nm to about 900 nm, about 5 nm to about 800 nm, about 5 nm to about 700 nm, about 5 nm to about 600 nm, about 5 nm to about 500 nm, about 5 nm to about 400 nm, about 5 nm to about 300 nm, about 5 nm to about 200 nm, about 5 nm to about 100 nm, about 5 nm to about 50 nm, about 5 nm to about 25 nm, about 5 nm to about 10 nm, about 10 nm to about 50 mm, about 10 nm to about 45 mm, about 10 nm to about 40 mm, about 10 nm to about 35 mm, about 10 nm to about 30 mm, about 10 nm to about 25 mm, about 10 nm to about 20 mm, about 10 nm to about 15 mm, about 10 nm to about 10 mm, about 10 nm to about 5 mm, about 10 nm to about 1 mm, about 10 nm to about 900 Tm, about 10 nm to about 800 Tm, about 10 nm to about 700 Tm, about 10 nm to about 600 Tm, about 10 nm to about 500 Tm, about 10 nm to about 400 Tm, about 10 nm to about 300 Tm, about 10 nm to about 200 Tm, about 10 nm to about 100 Tm, about 10 nm to about 50 Tm, about 10 nm to about 10 Tm, about 10 nm to about 5 Tm, about 10 nm to about 1 Tm, about 10 nm to about 900 nm, about 10 nm to about 800 nm, about 10 nm to about 700 nm, about 10 nm to about 600 nm, about 10 nm to about 500 nm, about 10 nm to about 400 nm, about 10 nm to about 300 nm, about 10 nm to about 200 nm, about 10 nm to about 100 nm, about 10 nm to about 50 nm, about 10 nm to about 25 nm, about 25 nm to about 50 mm, about 25 nm to about 45 mm, about 25 nm to about 40 mm, about 25 nm to about 35 mm, about 25 nm to about 30 mm, about 25 nm to about 25 mm, about 25 nm to about 20 mm, about 25 nm to about 15 mm, about 25 nm to about 10 mm, about 25 nm to about 5 mm, about 25 nm to about 1 mm, about 25 nm to about 900 Tm, about 25 nm to about 800 Tm, about 25 nm to about 700 Tm, about 25 nm to about 600 Tm, about 25 nm to about 500 Tm, about 25 nm to about 400 Tm, about 25 nm to about 300 Tm, about 25 nm to about 200 Tm, about 25 nm to about 100 Tm, about 25 nm to about 50 Tm, about 25 nm to about 10 Tm, about 25 nm to about 5 Tm, about 25 nm to about 1 Tm, about 25 nm to about 900 nm, about 25 nm to about 800 nm, about 25 nm to about 700 nm, about 25 nm to about 600 nm, about 25 nm to about 500 nm, about 25 nm to about 400 nm, about 25 nm to about 300 nm, about 25 nm to about 200 nm, about 25 nm to about 100 nm, about 25 nm to about 50 nm, about 50 nm to about 50 mm, about 50 nm to about 45 mm, about 50 nm to about 40 mm, about 50 nm to about 35 mm, about 50 nm to about 30 mm, about 50 nm to about 25 mm, about 50 nm to about 20 mm, about 50 nm to about 15 mm, about 50 nm to about 10 mm, about 50 nm to about 5 mm, about 50 nm to about 1 mm, about 50 nm to about 900 Tm, about 50 nm to about 800 Tm, about 50 nm to about 700 Tm, about 50 nm to about 600 Tm, about 50 nm to about 500 Tm, about 50 nm to about 400 Tm, about 50 nm to about 300 Tm, about 50 nm to about 200 Tm, about 50 nm to about 100 Tm, about 50 nm to about 50 Tm, about 50 nm to about 10 Tm, about 50 nm to about 5 Tm, about 50 nm to about 1 Tm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, about 50 nm to about 100 nm, about 100 nm to about 50 mm, about 100 nm to about 45 mm, about 100 nm to about 40 mm, about 100 nm to about 35 mm, about 100 nm to about 30 mm, about 100 nm to about 25 mm, about 100 nm to about 20 mm, about 100 nm to about 15 mm, about 100 nm to about 10 mm, about 100 nm to about 5 mm, about 100 nm to about 1 mm, about 100 nm to about 900 Tm, about 100 nm to about 800 Tm, about 100 nm to about 700 Tm, about 100 nm to about 600 Tm, about 100 nm to about 500 Tm, about 100 nm to about 400 Tm, about 100 nm to about 300 Tm, about 100 nm to about 200 Tm, about 100 nm to about 100 Tm, about 100 nm to about 50 Tm, about 100 nm to about 10 Tm, about 100 nm to about 5 Tm, about 100 nm to about 1 Tm, about 100 nm to about 900 nm, about 100 nm to about 800 nm, about 100 nm to about 700 nm, about 100 nm to about 600 nm, about 100 nm to about 500 nm, about 100 nm to about 400 nm, about 100 nm to about 300 nm, about 100 nm to about 200 nm, about 200 nm to about 50 mm, about 200 nm to about 45 mm, about 200 nm to about 40 mm, about 200 nm to about 35 mm, about 200 nm to about 30 mm, about 200 nm to about 25 mm, about 200 nm to about 20 mm, about 200 nm to about 15 mm, about 200 nm to about 10 mm, about 200 nm to about 5 mm, about 200 nm to about 1 mm, about 200 nm to about 900 Tm, about 200 nm to about 800 Tm, about 200 nm to about 700 Tm, about 200 nm to about 600 Tm, about 200 nm to about 500 Tm, about 200 nm to about 400 Tm, about 200 nm to about 300 Tm, about 200 nm to about 200 Tm, about 200 nm to about 100 Tm, about 200 nm to about 50 Tm, about 200 nm to about 10 Tm, about 200 nm to about 5 Tm, about 200 nm to about 1 Tm, about 200 nm to about 900 nm, about 200 nm to about 800 nm, about 200 nm to about 700 nm, about 200 nm to about 600 nm, about 200 nm to about 500 nm, about 200 nm to about 400 nm, about 200 nm to about 300 nm, about 300 nm to about 50 mm, about 300 nm to about 45 mm, about 300 nm to about 40 mm, about 300 nm to about 35 mm, about 300 nm to about 30 mm, about 300 nm to about 25 mm, about 300 nm to about 20 mm, about 300 nm to about 15 mm, about 300 nm to about 10 mm, about 300 nm to about 5 mm, about 300 nm to about 1 mm, about 300 nm to about 900 Tm, about 300 nm to about 800 Tm, about 300 nm to about 700 Tm, about 300 nm to about 600 Tm, about 300 nm to about 500 Tm, about 300 nm to about 400 Tm, about 300 nm to about 300 Tm, about 300 nm to about 200 Tm, about 300 nm to about 100 Tm, about 300 nm to about 50 Tm, about 300 nm to about 10 Tm, about 300 nm to about 5 Tm, about 300 nm to about 1 Tm, about 300 nm to about 900 nm, about 300 nm to about 800 nm, about 300 nm to about 700 nm, about 300 nm to about 600 nm, about 300 nm to about 500 nm, about 300 nm to about 400 nm, about 400 nm to about 50 mm, about 400 nm to about 45 mm, about 400 nm to about 40 mm, about 400 nm to about 35 mm, about 400 nm to about 30 mm, about 400 nm to about 25 mm, about 400 nm to about 20 mm, about 400 nm to about 15 mm, about 400 nm to about 10 mm, about 400 nm to about 5 mm, about 400 nm to about 1 mm, about 400 nm to about 900 Tm, about 400 nm to about 800 Tm, about 400 nm to about 700 Tm, about 400 nm to about 600 Tm, about 400 nm to about 500 Tm, about 400 nm to about 400 Tm, about 400 nm to about 300 Tm, about 400 nm to about 200 Tm, about 400 nm to about 100 Tm, about 400 nm to about 50 Tm, about 400 nm to about 10 Tm, about 400 nm to about 5 Tm, about 400 nm to about 1 Tm, about 400 nm to about 900 nm, about 400 nm to about 800 nm, about 400 nm to about 700 nm, about 400 nm to about 600 nm, about 400 nm to about 500 nm, about 500 nm to about 50 mm, about 500 nm to about 45 mm, about 500 nm to about 40 mm, about 500 nm to about 35 mm, about 500 nm to about 30 mm, about 500 nm to about 25 mm, about 500 nm to about 20 mm, about 500 nm to about 15 mm, about 500 nm to about 10 mm, about 500 nm to about 5 mm, about 500 nm to about 1 mm, about 500 nm to about 900 Tm, about 500 nm to about 800 Tm, about 500 nm to about 700 Tm, about 500 nm to about 600 Tm, about 500 nm to about 500 Tm, about 500 nm to about 400 Tm, about 500 nm to about 300 Tm, about 500 nm to about 200 Tm, about 500 nm to about 100 Tm, about 500 nm to about 50 Tm, about 500 nm to about 10 Tm, about 500 nm to about 5 Tm, about 500 nm to about 1 Tm, about 500 nm to about 900 nm, about 500 nm to about 800 nm, about 500 nm to about 700 nm, about 500 nm to about 600 nm, about 600 nm to about 50 mm, about 600 nm to about 45 mm, about 600 nm to about 40 mm, about 600 nm to about 35 mm, about 600 nm to about 30 mm, about 600 nm to about 25 mm, about 600 nm to about 20 mm, about 600 nm to about 15 mm, about 600 nm to about 10 mm, about 600 nm to about 5 mm, about 600 nm to about 1 mm, about 600 nm to about 900 Tm, about 600 nm to about 800 Tm, about 600 nm to about 700 Tm, about 600 nm to about 600 Tm, about 600 nm to about 500 Tm, about 600 nm to about 400 Tm, about 600 nm to about 300 Tm, about 600 nm to about 200 Tm, about 600 nm to about 100 Tm, about 600 nm to about 50 Tm, about 600 nm to about 10 Tm, about 600 nm to about 5 Tm, about 600 nm to about 1 Tm, about 600 nm to about 900 nm, about 600 nm to about 800 nm, about 600 nm to about 700 nm, about 700 nm to about 50 mm, about 700 nm to about 45 mm, about 700 nm to about 40 mm, about 700 nm to about 35 mm, about 700 nm to about 30 mm, about 700 nm to about 25 mm, about 700 nm to about 20 mm, about 700 nm to about 15 mm, about 700 nm to about 10 mm, about 700 nm to about 5 mm, about 700 nm to about 1 mm, about 700 nm to about 900 Tm, about 700 nm to about 800 Tm, about 700 nm to about 700 Tm, about 700 nm to about 600 Tm, about 700 nm to about 500 Tm, about 700 nm to about 400 Tm, about 700 nm to about 300 Tm, about 700 nm to about 200 Tm, about 700 nm to about 100 Tm, about 700 nm to about 50 Tm, about 700 nm to about 10 Tm, about 700 nm to about 5 Tm, about 700 nm to about 1 Tm, about 700 nm to about 900 nm, about 700 nm to about 800 nm, about 800 nm to about 50 mm, about 800 nm to about 45 mm, about 800 nm to about 40 mm, about 800 nm to about 35 mm, about 800 nm to about 30 mm, about 800 nm to about 25 mm, about 800 nm to about 20 mm, about 800 nm to about 15 mm, about 800 nm to about 10 mm, about 800 nm to about 5 mm, about 800 nm to about 1 mm, about 800 nm to about 900 Tm, about 800 nm to about 800 Tm, about 800 nm to about 700 Tm, about 800 nm to about 600 Tm, about 800 nm to about 500 Tm, about 800 nm to about 400 Tm, about 800 nm to about 300 Tm, about 800 nm to about 200 Tm, about 800 nm to about 100 Tm, about 800 nm to about 50 Tm, about 800 nm to about 10 Tm, about 800 nm to about 5 Tm, about 800 nm to about 1 Tm, about 800 nm to about 900 nm, about 900 nm to about 50 mm, about 900 nm to about 45 mm, about 900 nm to about 40 mm, about 900 nm to about 35 mm, about 900 nm to about 30 mm, about 900 nm to about 25 mm, about 900 nm to about 20 mm, about 900 nm to about 15 mm, about 900 nm to about 10 mm, about 900 nm to about 5 mm, about 900 nm to about 1 mm, about 900 nm to about 900 Tm, about 900 nm to about 800 Tm, about 900 nm to about 700 Tm, about 900 nm to about 600 Tm, about 900 nm to about 500 Tm, about 900 nm to about 400 Tm, about 900 nm to about 300 Tm, about 900 nm to about 200 Tm, about 900 nm to about 100 Tm, about 900 nm to about 50 Tm, about 900 nm to about 10 Tm, about 900 nm to about 5 Tm, about 900 nm to about 1 Tm, about 1 Tm to about 50 mm, about 1 Tm to about 45 mm, about 1 Tm to about 40 mm, about 1 Tm to about 35 mm, about 1 Tm to about 30 mm, about 1 Tm to about 25 mm, about 1 Tm to about 20 mm, about 1 Tm to about 15 mm, about 1 Tm to about 10 mm, about 1 Tm to about 5 mm, about 1 Tm to about 1 mm, about 1 Tm to about 900 Tm, about 1 Tm to about 800 Tm, about 1 Tm to about 700 Tm, about 1 Tm to about 600 Tm, about 1 Tm to about 500 Tm, about 1 Tm to about 400 Tm, about 1 Tm to about 300 Tm, about 1 Tm to about 200 Tm, about 1 Tm to about 100 Tm, about 1 Tm to about 50 Tm, about 1 Tm to about 10 Tm, about 1 Tm to about 5 Tm, about 5 Tm to about 50 mm, about 5 Tm to about 45 mm, about 5 Tm to about 40 mm, about 5 Tm to about 35 mm, about 5 Tm to about 30 mm, about 5 Tm to about 25 mm, about 5 Tm to about 20 mm, about 5 Tm to about 15 mm, about 5 Tm to about 10 mm, about 5 Tm to about 5 mm, about 5 Tm to about 1 mm, about 5 Tm to about 900 Tm, about 5 Tm to about 800 Tm, about 5 Tm to about 700 Tm, about 5 Tm to about 600 Tm, about 5 Tm to about 500 Tm, about 5 Tm to about 400 Tm, about 5 Tm to about 300 Tm, about 5 Tm to about 200 Tm, about 5 Tm to about 100 Tm, about 5 Tm to about 50 Tm, about 5 Tm to about 10 Tm, about 10 Tm to about 50 mm, about 10 Tm to about 45 mm, about 10 Tm to about 40 mm, about 10 Tm to about 35 mm, about 10 Tm to about 30 mm, about 10 Tm to about 25 mm, about 10 Tm to about 20 mm, about 10 Tm to about 15 mm, about 10 Tm to about 10 mm, about 10 Tm to about 5 mm, about 10 Tm to about 1 mm, about 10 Tm to about 900 Tm, about 10 Tm to about 800 Tm, about 10 Tm to about 700 Tm, about 10 Tm to about 600 Tm, about 10 Tm to about 500 Tm, about 10 Tm to about 400 Tm, about 10 Tm to about 300 Tm, about 10 Tm to about 200 Tm, about 10 Tm to about 100 Tm, about 10 Tm to about 50 Tm, about 50 Tm to about 50 mm, about 50 Tm to about 45 mm, about 50 Tm to about 40 mm, about 50 Tm to about 35 mm, about 50 Tm to about 30 mm, about 50 Tm to about 25 mm, about 50 Tm to about 20 mm, about 50 Tm to about 15 mm, about 50 Tm to about 10 mm, about 50 Tm to about 5 mm, about 50 Tm to about 1 mm, about 50 Tm to about 900 Tm, about 50 Tm to about 800 Tm, about 50 Tm to about 700 Tm, about 50 Tm to about 600 Tm, about 50 Tm to about 500 Tm, about 50 Tm to about 400 Tm, about 50 Tm to about 300 Tm, about 50 Tm to about 200 Tm, about 50 Tm to about 100 Tm, about 100 Tm to about 50 mm, about 100 Tm to about 45 mm, about 100 Tm to about 40 mm, about 100 Tm to about 35 mm, about 100 Tm to about 30 mm, about 100 Tm to about 25 mm, about 100 Tm to about 20 mm, about 100 Tm to about 15 mm, about 100 Tm to about 10 mm, about 100 Tm to about 5 mm, about 100 Tm to about 1 mm, about 100 Tm to about 900 Tm, about 100 Tm to about 800 Tm, about 100 Tm to about 700 Tm, about 100 Tm to about 600 Tm, about 100 Tm to about 500 Tm, about 100 Tm to about 400 Tm, about 100 Tm to about 300 Tm, about 100 Tm to about 200 Tm, about 200 Tm to about 50 mm, about 200 Tm to about 45 mm, about 200 Tm to about 40 mm, about 200 Tm to about 35 mm, about 200 Tm to about 30 mm, about 200 Tm to about 25 mm, about 200 Tm to about 20 mm, about 200 Tm to about 15 mm, about 200 Tm to about 10 mm, about 200 Tm to about 5 mm, about 200 Tm to about 1 mm, about 200 Tm to about 900 Tm, about 200 Tm to about 800 Tm, about 200 Tm to about 700 Tm, about 200 Tm to about 600 Tm, about 200 Tm to about 500 Tm, about 200 Tm to about 400 Tm, about 200 Tm to about 300 Tm, about 300 Tm to about 50 mm, about 300 Tm to about 45 mm, about 300 Tm to about 40 mm, about 300 Tm to about 35 mm, about 300 Tm to about 30 mm, about 300 Tm to about 25 mm, about 300 Tm to about 20 mm, about 300 Tm to about 15 mm, about 300 Tm to about 10 mm, about 300 Tm to about 5 mm, about 300 Tm to about 1 mm, about 300 Tm to about 900 Tm, about 300 Tm to about 800 Tm, about 300 Tm to about 700 Tm, about 300 Tm to about 600 Tm, about 300 Tm to about 500 Tm, about 300 Tm to about 400 Tm, about 400 Tm to about 50 mm, about 400 Tm to about 45 mm, about 400 Tm to about 40 mm, about 400 Tm to about 35 mm, about 400 Tm to about 30 mm, about 400 Tm to about 25 mm, about 400 Tm to about 20 mm, about 400 Tm to about 15 mm, about 400 Tm to about 10 mm, about 400 Tm to about 5 mm, about 400 Tm to about 1 mm, about 400 Tm to about 900 Tm, about 400 Tm to about 800 Tm, about 400 Tm to about 700 Tm, about 400 Tm to about 600 Tm, about 400 Tm to about 500 Tm, about 500 Tm to about 50 mm, about 500 Tm to about 45 mm, about 500 Tm to about 40 mm, about 500 Tm to about 35 mm, about 500 Tm to about 30 mm, about 500 Tm to about 25 mm, about 500 Tm to about 20 mm, about 500 Tm to about 15 mm, about 500 Tm to about 10 mm, about 500 Tm to about 5 mm, about 500 Tm to about 1 mm, about 500 Tm to about 900 Tm, about 500 Tm to about 800 Tm, about 500 Tm to about 700 Tm, about 500 Tm to about 600 Tm, about 600 Tm to about 50 mm, about 600 Tm to about 45 mm, about 600 Tm to about 40 mm, about 600 Tm to about 35 mm, about 600 Tm to about 30 mm, about 600 Tm to about 25 mm, about 600 Tm to about 20 mm, about 600 Tm to about 15 mm, about 600 Tm to about 10 mm, about 600 Tm to about 5 mm, about 600 Tm to about 1 mm, about 600 Tm to about 900 Tm, about 600 Tm to about 800 Tm, about 600 Tm to about 700 Tm, about 700 Tm to about 50 mm, about 700 Tm to about 45 mm, about 700 Tm to about 40 mm, about 700 Tm to about 35 mm, about 700 Tm to about 30 mm, about 700 Tm to about 25 mm, about 700 Tm to about 20 mm, about 700 Tm to about 15 mm, about 700 Tm to about 10 mm, about 700 Tm to about 5 mm, about 700 Tm to about 1 mm, about 700 Tm to about 900 Tm, about 700 Tm to about 800 Tm, about 800 Tm to about 50 mm, about 800 Tm to about 45 mm, about 800 Tm to about 40 mm, about 800 Tm to about 35 mm, about 800 Tm to about 30 mm, about 800 Tm to about 25 mm, about 800 Tm to about 20 mm, about 800 Tm to about 15 mm, about 800 Tm to about 10 mm, about 800 Tm to about 5 mm, about 800 Tm to about 1 mm, about 800 Tm to about 900 Tm, about 900 Tm to about 50 mm, about 900 Tm to about 45 mm, about 900 Tm to about 40 mm, about 900 Tm to about 35 mm, about 900 Tm to about 30 mm, about 900 Tm to about 25 mm, about 900 Tm to about 20 mm, about 900 Tm to about 15 mm, about 900 Tm to about 10 mm, about 900 Tm to about 5 mm, about 900 Tm to about 1 mm, about 1 mm to about 50 mm, about 1 mm to about 45 mm, about 1 mm to about 40 mm, about 1 mm to about 35 mm, about 1 mm to about 30 mm, about 1 mm to about 25 mm, about 1 mm to about 20 mm, about 1 mm to about 15 mm, about 1 mm to about 10 mm, about 1 mm to about 5 mm, about 1 mm to about 4 mm, about 1 mm to about 3 mm, about 1 mm to about 2 mm, about 5 mm to about 50 mm, about 5 mm to about 45 mm, about 5 mm to about 40 mm, about 5 mm to about 35 mm, about 5 mm to about 30 mm, about 5 mm to about 25 mm, about 5 mm to about 20 mm, about 5 mm to about 15 mm, about 5 mm to about 10 mm, about 10 mm to about 50 mm, about 10 mm to about 45 mm, about 10 mm to about 40 mm, about 10 mm to about 35 mm, about 10 mm to about 30 mm, about 10 mm to about 25 mm, about 10 mm to about 20 mm, about 10 mm to about 15 mm, about 15 mm to about 50 mm, about 15 mm to about 45 mm, about 15 mm to about 40 mm, about 15 mm to about 35 mm, about 15 mm to about 30 mm, about 15 mm to about 25 mm, about 15 mm to about 20 mm, about 20 mm to about 50 mm, about 20 mm to about 45 mm, about 20 mm to about 40 mm, about 20 mm to about 35 mm, about 20 mm to about 30 mm, about 20 mm to about 25 mm, about 25 mm to about 50 mm, about 25 mm to about 45 mm, about 25 mm to about 40 mm, about 25 mm to about 35 mm, about 25 mm to about 30 mm, about 30 mm to about 50 mm, about 30 mm to about 45 mm, about 30 mm to about 40 mm, about 30 mm to about 35 mm, about 35 mm to about 50 mm, about 35 mm to about 45 mm, about 35 mm to about 40 mm, about 40 mm to about 50 mm, about 40 mm to about 45 mm, or about 45 mm to about 50 mm.

In some embodiments, the bone graft material includes at least a portion of a fresh bone, freeze-dried bone, frozen bone, cadaveric bone, or a combination thereof. In some examples, the bone graft material is a cortical bone graft material. In some embodiments, the bone graft material is a cancellous bone graft material. In some embodiments, the bone graft material is a trabecular bone graft material. In some instances, the bone graft material is a periosteum bone graft material.

In some examples, the bone graft material is shaped as a disc, a wafer, a wedge, or a custom shape designed to fill a void in a bone. In some embodiments, the bone graft material is coated with one or more of the β-tricalcium phosphate (β-TCP)-binding sequence(s) disclosed herein and any one of the mammalian growth factors disclosed herein. In some embodiments, the bone graft material is coated with one or more of the β-tricalcium phosphate (β-TCP)-binding sequence(s) disclosed herein but does not include a mammalian growth factor. In some examples, a bone graft material shaped as a disc, a wafer, a wedge, or a custom shape designed to fill a void in a bone can be coated with one or more of the β-tricalcium phosphate (β-TCP)-binding sequence(s) disclosed herein and any one of the mammalian growth factors disclosed herein. In some embodiments, coating the graft material with one or more of the β-tricalcium phosphate (β-TCP)-binding sequence(s) disclosed herein and/or any one of the mammalian growth factors disclosed herein may enhance clinical performance (e.g., bone regrowth) and improve bio-functionality of the graft material as compared to the clinical performance and bio-functionality of graft materials that are not coated with one or more of the β-tricalcium phosphate (β-TCP)-binding sequence(s) disclosed herein and/or any one of the mammalian growth factors disclosed herein.

In some instances, the graft material is a soft tissue graft. In some embodiments, the soft tissue graft is a cartilage graft. In some embodiments, the soft tissue graft is a skin tissue, adipose tissue, placental tissue, amniotic membrane, tendon tissue, ligament tissue, gingival tissue, connective tissue, or any combination thereof.

In some examples, the graft material is a synthetic graft. In some embodiments, the synthetic graft includes a bone graft substitute. In some embodiments, the synthetic graft includes β-TCP, bioactive glass, calcium sulfate, tri-calcium phosphate ceramics, calcium phosphate cement, biphasic calcium phosphates, polymer-based bone subsitutes, or any combination thereof. Non-limiting examples of polymer-based bone subsitutes include nondegradable (e.g., poly(methylmethacrylate) or PMMA) or fully biodegradable, thus allowing a total bone replacement in time (e.g. polylactic acid (PLA)). In some embodiments, the graft material is a hybrid graft including one or more synthetic materials and one or more naturally-derived materials. In some embodiments, the graft material is a xenograft. In some embodiments, the xenograft is a porcine or bovine xenograft.

In some examples, the compositions can further include β-TCP (e.g., any of the types of β-TCP described herein). In some examples, the β-TCP is formulated as a powder, a putty (e.g., a moldable putty), a paste, a scaffold (e.g., a porous scaffold), a sponge, and/or a coating on a solid surface (e.g., a coating on a medical device). In some examples, the β-TCP can be disposed on or in a scaffold, a mesh, or a sponge (e.g., a resorbable sponge).

In some embodiments, the chimeric polypeptide is in contact with the graft material. For example, the chimeric polypeptide can be mixed in with the graft material to generate any of the variety of forms disclosed elsewhere herein. For example, the graft material may be coated with the chimeric polypeptide. For instance, the graft material may be a substrate or scaffold that may be coated with the chimeric polypeptide. In another embodiment, the graft material may be in a comminuted form and may further be partially or totally combined, mixed, blended, and/or amalgamated with the chimeric polypeptide. In yet another embodiment, a graft material in a comminuted form that has been partialy or totally combined, mixed, blended, and/or amalgamated with the chimeric polypeptide may be coated with the chimeric polypeptide after both components (i.e., the graft material and the chimeric polypeptide) have been partially or totally combined with one another.

In some instances, the compositions (e.g., pharmaceutical compositions) are disposed in a sterile vial or a pre-loaded syringe.

In some instances, the compositions (e.g., pharmaceutical compositions) are formulated for different routes of administration (e.g., intraarticular, injection into a joint, or injection proximal to a bone fissue or fracture). Single or multiple administrations of any of the pharmaceutical compositions described herein can be given to a subject depend on, for example: the dosage and frequency as required and tolerated by the subject. A dosage of the pharmaceutical composition should provide a sufficient quantity of the chimeric polypeptide to effective treat or ameliorate conditions (e.g., bone defects, bone fractures, cartilage defects, or cartilage loss), or symptoms.

Also provided herein are kits that include any of the chimeric polypeptides or any of the β-TCP-binding polypeptides described herein, or any of the compositions (e.g., pharmaceutical compositions) described herein. In some embodiments, the kits can include instructions for performing any of the methods described herein. In some instances, the kits can include at least one dose of any of the compositions (e.g., pharmaceutical compositions) described herein. In some embodiments, the kits can include a syringe for administering any of the pharmaceutical compositions described herein. The kits described herein are not so limited; other variations will be apparent to one of ordinary skill in the art.

### Methods of Making a Composition

Also provided herein are methods of producing any of the compositions described herein. Any of the compositions provided herein can be produced using the methods described herein or methods known in the art. For example, to create a composition disclosed herein (e.g., a substrate or a scaffold), granulated graft material can be combined with one or more of the chimeric polypeptides described herein, sintered, sieved, and fabricated into a desired shape (e.g., any of the shapes described herein). In another embodiment, to create a composition disclosed herein, the graft material can be sintered, sieved, and fabricated into a desired shape (e.g., any of the shapes described herein), and then coated with one or more of the chimeric polypeptides described herein. In yet another embodiment, the compositions can be incorporated with one or more of the chimeric polypeptides disclosed herein and/or coated with one or more of the chimeric polypeptides disclosed herein.

In some embodiments, the graft material can include hydroxyapatite. In some embodiments, the graft material can include β-TCP. In some examples, the purity of the β-TCP present in any of the compositions described herein can be greater than about 75%, greater than 80%, greater than 85%, greater than 90%, greater than 95%, or greater than 99% pure. In some examples, the purity of β-TCP present in any of the compositions described herein can be greater than 1%, greater than 2%, greater than 3%, greater than 4%, greater than 5%, greater than 6%, greater than 7%, greater than 8%, greater than 9%, greater than 10%, greater than 12%, greater than 14%, greater than 15%, greater than 16%, greater than 18%, greater than 20%, greater than 22%, greater than 24%, greater than 25%, greater than 26%, greater than 28%, greater than 30%, greater than 32%, greater than 34%, greater than 35%, greater than 36%, greater 38%, greater 40%, greater than 42%, greater than 44%, greater than 45%, greater than 46%, greater than 48%, greater than 50%, greater than 52%, greater than 54%, greater than 55%, greater than 56%, greater than 58%, greater than 60%, greater than 62%, greater than 64%, greater than 65%, greater than 66%, greater than 68%, greater than 70%, greater than 72%, greater than 74%. In some examples, the β-TCP is made using a similar method but as a composite with other agents, such as a biocompatible polymer, e.g., polylactide-co-glycolide.

As will be apparent to those of skill in the art, the composition can further include one or more pore forming agents. Examples of pore forming agents include, e.g., inorganic salts, such as sodium chloride, saccharides (e.g., sucrose or glucose), gelatin (e.g., gelatin spheres), or paraffin (e.g., paraffin spheres).

The compositions described herein can be generated by contacting any of the chimeric polypeptides or any of the β-TCP-binding polypeptides to any of the types of graft materials described herein. In some embodiments, the graft material can be in the form of a granular/powder form, a porous form, a putty (e.g., a moldable putty), a paste, a scaffold, and/or a coating on a solid surface (e.g., a coating on a medical device).

### Methods of Treatment

Provided herein are methods of promoting bone or cartilage formation in a subject in need thereof that include: administering to the subject a therapeutically effective amount of any of the compositions described herein. Some embodiments of these methods can further include first selecting a subject in need of bone or cartilage formation. In some embodiments, the composition is administered to the subject proximal to the desired site of bone or cartilage formation in the subject.

Also provided herein are methods of replacing and/or repairing bone or cartilage in a subject in need thereof that include: administering to the subject a therapeutically effective amount of any of the compositions described herein. Some embodiments of these methods can further include first selecting a subject in need of bone replacement, bone repair, cartilage replacement, or cartilage repair. In some embodiments, the composition is administered to the subject proximal to the desired site of bone or cartilage replacement or repair in the subject.

Also provided herein are methods of treating a bone fracture or bone loss in a subject in need thereof, the method including administering to the subject a therapeutically effective amount of any of the compositions described herein. Some embodiments of these methods can further include first selecting a subject having a bone fracture or bone loss. In some embodiments, the composition is administered to the subject proximal to the bone fracture or the site of bone loss in the subject.

Also provided herein are methods of repairing soft tissue in a subject in need thereof that include: administering to the subject a therapeutically effective amount of any of the compositions described herein. Some embodiments of these methods can further include first selecting a subject having a bone fracture or bone loss. In some embodiments, the composition is administered to the subject proximal to the bone fracture or the site of bone loss in the subject.

Also provided herein are methods of localized delivery of a therapeutic to a subject in need thereof that include: administering to the subject a therapeutically effective amount of any of the compositions described herein. Some embodiments of these methods can further include first selecting a subject having a bone fracture or bone loss. In some embodiments, the composition is administered to the subject proximal to the bone fracture or the site of bone loss in the subject.

In some instances, the subject has a bone fracture or a bone defect.

In some instances, the subject requires a vertebral fusion of the spine.

In some instances, the subject has a cartilage tear or cartilage defect.

In other instances, the subject has cartilage loss.

Methods of determining the efficacy of treatment of a bone fracture or bone loss in a subject are known in the art and include, e.g., imaging techniques (e.g., magnetic resonance imaging, X-ray, or computed tomography).

Methods of detecting bone or cartilage formation, or replacement or repair of bone or cartilage in a subject are also known in the art and include, e.g., imaging techniques (e.g., magnetic resonance imaging, X-ray, or computed tomography).

Suitable animal models for treatment of a bone fraction or bone loss, bone or cartilage formation, or bone or cartilage replacement or repair are known in the art. Non-limiting examples of such animal models are described in the Examples and in, e.g., Drosse et al., Tissue Engineering Part C 14(1):79-88, 2008; Histing et al., Bone 49:591-599, 2011; and Poser et al., Hindawi Publishing Corporation, BioMed Research International; Article ID 348635, 2014.

### Chimeric Polypeptides

The chimeric polypeptides described herein include a β-TCP binding sequence and can bind β-TCP (e.g., any of the exemplary types of β-TCP described herein or known in the art). In some examples, the chimeric polypeptides can include: (i) one or more (e.g., two, three, four, five, six, seven, eight, nine, or ten) β-tricalcium phosphate (βTCP)-binding sequence(s), where at least one of the one or more β-TCP-binding sequence(s) is selected from the group of: VIGESTHHRPWS (SEQ ID NO: 2), LIADSTHHSPWT (SEQ ID NO: 3), ILAESTHHKPWT (SEQ ID NO: 4), ILAETTHHRPWS (SEQ ID NO: 5), IIGESSHHKPFT (SEQ ID NO: 6), GLGDTTHHRPWG (SEQ ID NO: 7), VLGDTTHHKPWT (SEQ ID NO: 8), IVADSTHHRPWT (SEQ ID NO: 9); STADTSHHRPS (SEQ ID NO: 10), TSGGESTHHRPS (SEQ ID NO: 11), TSGGESSHHKPS (SEQ ID NO: 12), TGSGDSSHHRPS (SEQ ID NO: 13), GSSGESTHHKPST (SEQ ID NO: 14), VGADSTHHRPVT (SEQ ID NO: 15), GAADTTHHRPVT (SEQ ID NO: 16), AGADTTHHRPVT (SEQ ID NO: 17), GGADTTHHRPAT (SEQ ID NO: 18), and GGADTTHHRPGT (SEQ ID NO: 19); and (ii) a mammalian growth factor (e.g., any of the exemplary mammalian growth factors described herein or known in the art).

In some examples, the chimeric polypeptides can include: (i) one or more β-TCP-binding sequence(s) including an amino acid sequence of Formula I:
A₀B₀C₀D₀E₀F₀G₀H₀I₀J₀K₀L₀ (Formula I) (SEQ ID NO: 35), wherein:
   A₀ is V, L, I, G, S, T, or A;
   B₀ is I, L, V, Q, T, S, G, or A;
   C₀ is G, A, V, or S;
   D₀ is E, D, L, or G;
   E₀ is S, T, P T, E, or D;
   F₀ is T or S;
   G₀ is H, T, or S;
   H₀ is H or T;
   I₀ is R, S, K, P, or H;
   J₀ is P, S, R, or K;
   K₀ is W, F, S, P, V, A, or G; and
   L₀ is absent or is S, T, G, or A; and (ii) a mammalian growth factor (e.g., any of the exemplary mammalian growth factors described herein or known in the art), where Formula I does not include LLADTTHHRPWT (SEQ ID NO: 1).

Non-limiting examples of β-TCP binding sequences that may be present in any of the chimeric polypeptides described herein are listed in Table A below.

TABLE A

Exemplary β-TCP binding sequences

| P-TCP binding sequence | SEQ ID NO: |
|---|---|
| LLADTTHHRPWT | 1 |
| VIGESTHHRPWS | 2 |
| LIADSTHHSPWT | 3 |
| ILAESTHHKPWT | 4 |
| ILAETTHHRPWS | 5 |
| IIGESSHHKPFT | 6 |
| GLGDTTHHRPWG | 7 |
| VLGDTTHHKPWT | 8 |
| IVADSTHHRPWT | 9 |
| STADTSHHRPS | 10 |
| TSGGESTHHRPS | 11 |
| TSGGESSHHKPS | 12 |
| TGSGDSSHHRPS | 13 |
| GSSGESTHHKPST | 14 |
| VGADSTHHRPVT | 15 |
| GAADTTHHRPVT | 16 |
| AGADTTHHRPVT | 17 |
| GGADTTHHRPAT | 18 |
| GGADTTHHRPGT | 19 |
| LLADTTHHRPWTVIGESTHHRPWS | 20 |
| LLADTTHHRPWTVIGESTHHRPWSIIGESSHHKPFT | 21 |
| LLADTTHHRPWTVIGESTHHRPWSIIGESSHHKPFTGLGD TTHHRPWGILAESTHHKPWT | 22 |
| LLADTTHHRPWTILAESTHHKPWT | 23 |
| LLADTTHHRPWTILAESTHHKPWTLLADTTHHRPWT ILAESTHHKPWTLLADTTHHRPWT | 24 |
| LLADTTHHRPWTGLGDTTHHRPWG | 25 |
| LLADTTHHRPWTGLGDTTHHRPWGLLADTTHHRPWT | 26 |
| LLADTTHHRPWTGLGDTTHHRPWGLLADTTHHRPWTGLGDTTHHRPWGLLADTTHHRPWT | 27 |
| LLADTTHHRPWTGLGDTTHHRPWGLLADTTHHRPWTGLGDTTHHRPWGLLADTTHHRPWTGLGDTTHHRPWGLLADTTHHRPWT | 28 |
| STADTSHHRPSTSGGESTHHRPSTSGGESSHHKPSTGSGDSSHHRPSGSSGESTHHKPST | 29 |
| VGADSTHHRPVTGAADTTHHRPVTAGADTTHHRPVTGGADTTHHRPATGGADTTHHRPGT | 30 |
| STADTSHHRPSLLADTTHHRPWTTSGGESTHHRPSVGADSTHHRPVT TSGGESSHHKPSGAADTTHHRPVTTGSGDSSHHRPSGSSGESTHHKPS TGGADTTHHRPAT | 31 |
| AAADTTHHRPWT | 36 |
| AAADTTHHRPWTAAADTTHHRPWTAAADTTHHRPWTAAADTTHHRPWTAAADTTHHRPWT | 37 |
| LLADAAHHRPWTLLADAAHHRPWTLLADAAHHRPWTLLADAAHHRPWTLLADAAHHRPWT | 38 |
| LLADTTAARPWTLLADTTAARPWTLLADTTAARPWT LLADTTAARPWTLLADTTAARPWT | 39 |
| LLADTTHHRPWTLLADTTHHRPWT | 40 |
| LLADTTHHRPWTLLADTTHHRPWTLLADTTHHRPWT | 41 |
| LLADTTHHRPWTLLADTTHHRPWTLLADTTHHRPWTLLADTTHHRPWTLLADTTHHRPWT | 42 |
| STSGSTVIGESTHHRPWSLIADSTHHSPWTILAESTHHKPWTILAETTHHRPWSIIGESSHHKPFTGLGDTTHHRPWGVLGDTTHHKPWTIVADSTHHRPWTGQVLPTTTPSSPSTTSGS | 43 |

In some embodiments, any of the chimeric polypeptides described herein can includes two or more (e.g., three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more; or 2, 3, 4, 5, 6, 7, 8, 9, 10, 11, 12, 13, 14, 15, 16, 17, 18, 19, 20, 21, or 22) β-TCP-binding sequences (e.g., any of the exemplary β-TCP-binding sequences described herein). In some embodiments, each neighboring pair of the two or more β-TCP binding sequences directly abut each other. In some embodiments, each neighboring pair of the two or more β-TCP binding sequences (e.g., any of the exemplary β-TCP-binding sequences described herein) are separated by a linker sequence.

In some embodiments, the chimeric polypeptides can include at least two (e.g., at least three, at least four, at least five, at least six, at least seven, at least eight, at least nine, or at least ten; or 2, 3, 4, 5, 6, 7, 8, 9, or 10) different β-TCP-binding sequences (e.g., any of the exemplary β-TCP-binding sequences described herein).

In some embodiments, at least one of the two or more different β-TCP-binding sequences is LLADTTHHRPWT (SEQ ID NO: 1) or GQVLPTTTPSSP (SEQ ID NO: 19). In some embodiments, the at least two different β-TCP-binding sequences include LLADTTHHRPWT (SEQ ID NO: 1) and VIGESTHHRPWS (SEQ ID NO: 2). In some embodiments, the chimeric polypeptide includes the sequence of LLADTTHHRPWTVIGESTHHRPWS (SEQ ID NO: 20).

In some embodiments, the at least two different β-TCP-binding sequences include LLADTTHHRPWT (SEQ ID NO: 1), VIGESTHHRPWS (SEQ ID NO: 2), and IIGESSHHKPFT (SEQ ID NO: 6). In some embodiments, the chimeric polypeptide includes the sequence of LLADTTHHRPWTVIGESTHHRPWSIIGESSHHKPFT (SEQ ID NO: 21).

In some embodiments, the at least two different β-TCP-binding sequences include LLADTTHHRPWT (SEQ ID NO: 1), VIGESTHHRPWS (SEQ ID NO: 2), IIGESSHHKPFT (SEQ ID NO: 6), GLGDTTHHRPWG (SEQ ID NO: 7), and ILAESTHHKPWT (SEQ ID NO: 4). In some embodiments, the chimeric polypeptide includes the sequence of: LLADTTHHRPWTVIGESTHHRPWSIIGESSHHKPFTGLGDTTHHRPWGILAESTHHKPWT (SEQ ID NO: 22).

In some embodiments, the at least two β-TCP-binding sequences include LLADTTHHRPWT (SEQ ID NO: 1) and ILAESTHHKPWT (SEQ ID NO: 4). In some embodiments, the chimeric polypeptide includes at least one copy of the sequence LLADTTHHRPWTILAESTHHKPWT (SEQ ID NO: 23). In some embodiments, the chimeric polypeptide includes the sequence of LLADTTHHRPWTILAESTHHKPWTLLADTTHHRPWTILAESTHHKPWTLLADTTHHRPWT (SEQ ID NO: 24).

In some embodiments, the at least two β-TCP-binding sequences include LLADTTHHRPWT (SEQ ID NO: 1) and GLGDTTHHRPWG (SEQ ID NO: 7). In some embodiments, the chimeric polypeptide includes at least one copy of the sequence of LLADTTHHRPWTGLGDTTHHRPWG (SEQ ID NO: 25). In some embodiments, the chimeric polypeptide includes the sequence of LLADTTHHRPWTGLGDTTHHRPWGLLADTTHHRPWT (SEQ ID NO: 26). In some embodiments, the chimeric polypeptide includes the sequence of LLADTTHHRPWTGLGDTTHHRPWGLLADTTHHRPWTGLGDTTHHRPWGLLADTTHHRPWT (SEQ ID NO: 27). In some embodiments, the chimeric polypeptide includes the sequence of LLADTTHHRPWTGLGDTTHHRPWGLLADTTHHRPWTGLGDTTHHRPWGLLADTTHHRPWTGLGDTTHHRPWGLLADTTHHRPWT (SEQ ID NO: 28).

In some embodiments, the at least two different β-TCP-binding sequences include STADTSHHRPS (SEQ ID NO: 10), TSGGESTHHRPS (SEQ ID NO: 11), TSGGESSHHKPS (SEQ ID NO: 12), TGSGDSSHHRPS (SEQ ID NO: 13), and GSSGESTHHKPST (SEQ ID NO: 14). In some embodiments, the chimeric polypeptide includes the sequence of STADTSHHRPSTSGGESTHHRPSTSGGESSHHKPSTGSGDSSHHRPSGSSGESTHHKPST (SEQ ID NO: 29).

In some embodiments, the at least two different β-TCP-binding sequences include VGADSTHHRPVT (SEQ ID NO: 15), GAADTTHHRPVT SEQ ID NO: 16), AGADTTHHRPVT (SEQ ID NO: 17), GGADTTHHRPAT (SEQ ID NO: 18) and GGADTTHHRPGT (SEQ ID NO: 19). In some embodiments, the chimeric polypeptide includes the sequence of VGADSTHHRPVTGAADTTHHRPVTAGADTTHHRPVTGGADTTHHRPATGGADTTHHRPGT (SEQ ID NO: 30).

In some embodiments, the at least two different β-TCP-binding sequences include STADTSHHRPS (SEQ ID NO: 10), LLADTTHHRPWT (SEQ ID NO: 1),TSGGESTHHRPS (SEQ ID NO: 11), VGADSTHHRPVT (SEQ ID NO: 15),TSGGESSHHKPS (SEQ ID NO: 12), GAADTTHHRPVT (SEQ ID NO: 16),TGSGDSSHHRPS (SEQ ID NO: 13), GSSGESTHHKPS (SEQ ID NO: 14), and TGGADTTHHRPAT (SEQ ID NO: 18). In some embodiments, the chimeric polypeptide includes the sequence of: STADTSHHRPSLLADTTHHRPWTTSGGESTHHRPSVGADSTHHRPVTTSGGESSHHKPSGAADTTHHRPTGSGDSSHHRPSGSSGESTHHKPSTGGADTTHHRPAT (SEQ ID NO: 31).

In some embodiments, the chimeric polypeptides can include two or more (e.g., three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more; or 2, 3, 4, 5, 6, 7, 8, 9, 10, 11, 12, 13, 14, 15, 16, 17, 18, 19, or 20) copies of the same β-TCP-binding sequence (e.g., any of the exemplary β-TCP-binding sequences described herein).

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 1.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 2.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 3.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 4.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 5.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 6.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 7.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 8.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 9.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 10.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 11.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 12.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 13.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 14.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 15.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 16.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 17.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 18.

In some embodiments, the chimeric polypeptides can include a β-TCP-binding sequence that has or includes an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 19.

In some embodiments, the chimeric polypeptide further includes a linker sequence (e.g., any of the exemplary linker sequences described herein or known in the art) between a neighboring pair of a β-TCP-binding sequence (e.g., any of the exemplary β-TCP binding sequences described herein) and the mammalian growth factor (e.g., any of the exemplary mammalian growth factor described herein). In some embodiments, the linker sequence can include the sequence of TGGSGEGGTGASTGGSAGTGGSGGTTSGEAGGSSGAG (SEQ ID NO: 33). In some embodiments, the linker sequence consists of TGGSGEGGTGASTGGSAGTGGSGGTTSGEAGGSSGAG (SEQ ID NO: 33). In some embodiments, the linker sequence includes the sequence of GSGATG (SEQ ID NO: 34). In some embodiments, the linker sequence consists of GSGATG (SEQ ID NO: 34).

In some embodiments, where the chimeric polypeptide includes two or more β-TCP-binding sequences, a linker sequence (e.g., any of the exemplary linker sequences described herein or known in the art) can be between any of the pairs (e.g., all of the pairs) of neighboring β-TCP-binding sequences.

In some embodiments, the chimeric polypeptide can include a signal sequence at its N-terminus. In some embodiments, the chimeric polypeptide can further include a tag sequence (e.g., a poly-His tag, chitin-binding protein (CBP), maltose-binding protein (MBP), strep-tag, glutathione-S-transferase (GST), thioredoxin, or Fc region). Additional examples of tags are known in the art.

The chimeric polypeptide described herein can bind to β-TCP (e.g., any of the types of β-TCP described herein) with a dissociation equilibrium constant (K_{d}) of about 1 pM to about 100 µM (e.g., about 1 pM to about 95 µM, about 1 pM to about 90 µM, about 1 pM to about 85 µM, about 1 pM to about 80 µM, about 1 pM to about 75 µM, about 1 pM to about 70 µM, about 1 pM to about 65 µM, about 1 pM to about 60 µM, about 1 pM to about 55 µM, about 1 pM to about 50 µM, about 1 pM to about 45 µM, about 1 pM to about 40 µM, about 1 pM to about 45 µM, about 1 pM to about 40 µM, about 1 pM to about 35 µM, about 1 pM to about 30 µM, about 1 pM to about 25 µM, about 1 pM to about 20 µM, about 1 pM to about 15 µM, about 1 pM to about 10 µM, about 1 pM to about 5 µM, about 1 pM to about 2 µM, about 1 pM to about 1 µM, about 1 pM to about 950 nM, about 1 pM to about 900 nM, about 1 pM to about 850 nM, about 1 pM to about 800 nM, about 1 pM to about 750 nM, about 1 pM to about 700 nM, about 1 pM to about 650 nM, about 1 pM to about 600 nM, about 1 pM to about 550 nM, about 1 pM to about 500 nM, about 1 pM to about 450 nM, about 1 pM to about 400 nM, about 1 pM to about 350 nM, about 1 pM to about 300 nM, about 1 pM to about 250 nM, about 1 pM to about 200 nM, about 1 pM to about 150 nM, about 1 pM to about 100 nM, about 1 pM to about 95 nM, about 1 pM to about 90 nM, about 1 pM to about 85 nM, about 1 pM to about 80 nM, about 1 pM to about 75 nM, about 1 pM to about 70 nM, about 1 pM to about 75 nM, about 1 pM to about 70 nM, about 1 pM to about 65 nM, about 1 pM to about 60 nM, about 1 pM to about 55 nM, about 1 pM to about 50 nM, about 1 pM to about 45 nM, about 1 pM to about 40 nM, about 1 pM to about 35 nM, about 1 pM to about 30 nM, about 1 pM to about 25 nM, about 1 pM to about 20 nM, about 1 pM to about 15 nM, about 1 pM to about 10 nM, about 1 pM to about 5 nM, about 1 pM to about 2 nM, about 1 pM to about 1 nM, about 1 pM to about 950 pM, about 1 pM to about 900 pM, about 1 pM to about 850 pM, about 1 pM to about 800 pM, about 1 pM to about 750 pM, about 1 pM to about 700 pM, about 1 pM to about 650 pM, about 1 pM to about 600 pM, about 1 pM to about 550 pM, about 1 pM to about 500 pM, about 1 pM to about 450 pM, about 1 pM to about 400 pM, about 1 pM to about 350 pM, about 1 pM to about 300 pM, about 1 pM to about 250 pM, about 1 pM to about 200 pM, about 1 pM to about 150 pM, about 1 pM to about 100 pM, about 1 pM to about 95 pM, about 1 pM to about 90 pM, about 1 pM to about 85 pM, about 1 pM to about 80 pM, about 1 pM to about 75 pM, about 1 pM to about 70 pM, about 1 pM to about 75 pM, about 1 pM to about 70 pM, about 1 pM to about 65 pM, about 1 pM to about 60 pM, about 1 pM to about 55 pM, about 1 pM to about 50 pM, about 1 pM to about 45 pM, about 1 pM to about 40 pM, about 1 pM to about 35 pM, about 1 pM to about 30 pM, about 1 pM to about 25 pM, about 1 pM to about 20 pM, about 1 pM to about 15 pM, about 1 pM to about 10 pM, about 1 pM to about 5 pM, about 1 pM to about 2 pM,. about 2 pM to about 100 µM, about 2 pM to about 95 µM, about 2 pM to about 90 µM, about 2 pM to about 85 µM, about 2 pM to about 80 µM, about 2 pM to about 75 µM, about 2 pM to about 70 µM, about 2 pM to about 65 µM, about 2 pM to about 60 µM, about 2 pM to about 55 µM, about 2 pM to about 50 µM, about 2 pM to about 45 µM, about 2 pM to about 40 µM, about 2 pM to about 45 µM, about 2 pM to about 40 µM, about 2 pM to about 35 µM, about 2 pM to about 30 µM, about 2 pM to about 25 µM, about 2 pM to about 20 µM, about 2 pM to about 15 µM, about 2 pM to about 10 µM, about 2 pM to about 5 µM, about 2 pM to about 2 µM, about 2 pM to about 1 µM, about 2 pM to about 950 nM, about 2 pM to about 900 nM, about 2 pM to about 850 nM, about 2 pM to about 800 nM, about 2 pM to about 750 nM, about 2 pM to about 700 nM, about 2 pM to about 650 nM, about 2 pM to about 600 nM, about 2 pM to about 550 nM, about 2 pM to about 500 nM, about 2 pM to about 450 nM, about 2 pM to about 400 nM, about 2 pM to about 350 nM, about 2 pM to about 300 nM, about 2 pM to about 250 nM, about 2 pM to about 200 nM, about 2 pM to about 150 nM, about 2 pM to about 100 nM, about 2 pM to about 95 nM, about 2 pM to about 90 nM, about 2 pM to about 85 nM, about 2 pM to about 80 nM, about 2 pM to about 75 nM, about 2 pM to about 70 nM, about 2 pM to about 75 nM, about 2 pM to about 70 nM, about 2 pM to about 65 nM, about 2 pM to about 60 nM, about 2 pM to about 55 nM, about 2 pM to about 50 nM, about 2 pM to about 45 nM, about 2 pM to about 40 nM, about 2 pM to about 35 nM, about 2 pM to about 30 nM, about 2 pM to about 25 nM, about 2 pM to about 20 nM, about 2 pM to about 15 nM, about 2 pM to about 10 nM, about 2 pM to about 5 nM, about 2 pM to about 2 nM, about 2 pM to about 1 nM, about 2 pM to about 950 pM, about 2 pM to about 900 pM, about 2 pM to about 850 pM, about 2 pM to about 800 pM, about 2 pM to about 750 pM, about 2 pM to about 700 pM, about 2 pM to about 650 pM, about 2 pM to about 600 pM, about 2 pM to about 550 pM, about 2 pM to about 500 pM, about 2 pM to about 450 pM, about 2 pM to about 400 pM, about 2 pM to about 350 pM, about 2 pM to about 300 pM, about 2 pM to about 250 pM, about 2 pM to about 200 pM, about 2 pM to about 150 pM, about 2 pM to about 100 pM, about 2 pM to about 95 pM, about 2 pM to about 90 pM, about 2 pM to about 85 pM, about 2 pM to about 80 pM, about 2 pM to about 75 pM, about 2 pM to about 70 pM, about 2 pM to about 75 pM, about 2 pM to about 70 pM, about 2 pM to about 65 pM, about 2 pM to about 60 pM, about 2 pM to about 55 pM, about 2 pM to about 50 pM, about 2 pM to about 45 pM, about 2 pM to about 40 pM, about 2 pM to about 35 pM, about 2 pM to about 30 pM, about 2 pM to about 25 pM, about 2 pM to about 20 pM, about 2 pM to about 15 pM, about 2 pM to about 10 pM, about 2 pM to about 5 pM, about 5 pM to about 100 µM, about 5 pM to about 95 µM, about 5 pM to about 90 µM, about 5 pM to about 85 µM, about 5 pM to about 80 µM, about 5 pM to about 75 µM, about 5 pM to about 70 µM, about 5 pM to about 65 µM, about 5 pM to about 60 µM, about 5 pM to about 55 µM, about 5 pM to about 50 µM, about 5 pM to about 45 µM, about 5 pM to about 40 µM, about 5 pM to about 45 µM, about 5 pM to about 40 µM, about 5 pM to about 35 µM, about 5 pM to about 30 µM, about 5 pM to about 25 µM, about 5 pM to about 20 µM, about 5 pM to about 15 µM, about 5 pM to about 10 µM, about 5 pM to about 5 µM, about 5 pM to about 2 µM, about 5 pM to about 1 µM, about 5 pM to about 950 nM, about 5 pM to about 900 nM, about 5 pM to about 850 nM, about 5 pM to about 800 nM, about 5 pM to about 750 nM, about 5 pM to about 700 nM, about 5 pM to about 650 nM, about 5 pM to about 600 nM, about 5 pM to about 550 nM, about 5 pM to about 500 nM, about 5 pM to about 450 nM, about 5 pM to about 400 nM, about 5 pM to about 350 nM, about 5 pM to about 300 nM, about 5 pM to about 250 nM, about 5 pM to about 200 nM, about 5 pM to about 150 nM, about 5 pM to about 100 nM, about 5 pM to about 95 nM, about 5 pM to about 90 nM, about 5 pM to about 85 nM, about 5 pM to about 80 nM, about 5 pM to about 75 nM, about 5 pM to about 70 nM, about 5 pM to about 75 nM, about 5 pM to about 70 nM, about 5 pM to about 65 nM, about 5 pM to about 60 nM, about 5 pM to about 55 nM, about 5 pM to about 50 nM, about 5 pM to about 45 nM, about 5 pM to about 40 nM, about 5 pM to about 35 nM, about 5 pM to about 30 nM, about 5 pM to about 25 nM, about 5 pM to about 20 nM, about 5 pM to about 15 nM, about 5 pM to about 10 nM, about 5 pM to about 5 nM, about 5 pM to about 2 nM, about 5 pM to about 1 nM, about 5 pM to about 950 pM, about 5 pM to about 900 pM, about 5 pM to about 850 pM, about 5 pM to about 800 pM, about 5 pM to about 750 pM, about 5 pM to about 700 pM, about 5 pM to about 650 pM, about 5 pM to about 600 pM, about 5 pM to about 550 pM, about 5 pM to about 500 pM, about 5 pM to about 450 pM, about 5 pM to about 400 pM, about 5 pM to about 350 pM, about 5 pM to about 300 pM, about 5 pM to about 250 pM, about 5 pM to about 200 pM, about 5 pM to about 150 pM, about 5 pM to about 100 pM, about 5 pM to about 95 pM, about 5 pM to about 90 pM, about 5 pM to about 85 pM, about 5 pM to about 80 pM, about 5 pM to about 75 pM, about 5 pM to about 70 pM, about 5 pM to about 75 pM, about 5 pM to about 70 pM, about 5 pM to about 65 pM, about 5 pM to about 60 pM, about 5 pM to about 55 pM, about 5 pM to about 50 pM, about 5 pM to about 45 pM, about 5 pM to about 40 pM, about 5 pM to about 35 pM, about 5 pM to about 30 pM, about 5 pM to about 25 pM, about 5 pM to about 20 pM, about 5 pM to about 15 pM, about 5 pM to about 10 pM, about 10 pM to about 100 µM, about 10 pM to about 95 µM, about 10 pM to about 90 µM, about 10 pM to about 85 µM, about 10 pM to about 80 µM, about 10 pM to about 75 µM, about 10 pM to about 70 µM, about 10 pM to about 65 µM, about 10 pM to about 60 µM, about 10 pM to about 55 µM, about 10 pM to about 50 µM, about 10 pM to about 45 µM, about 10 pM to about 40 µM, about 10 pM to about 45 µM, about 10 pM to about 40 µM, about 10 pM to about 35 µM, about 10 pM to about 30 µM, about 10 pM to about 25 µM, about 10 pM to about 20 µM, about 10 pM to about 15 µM, about 10 pM to about 10 µM, about 10 pM to about 5 µM, about 10 pM to about 2 µM, about 10 pM to about 1 µM, about 10 pM to about 950 nM, about 10 pM to about 900 nM, about 10 pM to about 850 nM, about 10 pM to about 800 nM, about 10 pM to about 750 nM, about 10 pM to about 700 nM, about 10 pM to about 650 nM, about 10 pM to about 600 nM, about 10 pM to about 550 nM, about 10 pM to about 500 nM, about 10 pM to about 450 nM, about 10 pM to about 400 nM, about 10 pM to about 350 nM, about 10 pM to about 300 nM, about 10 pM to about 250 nM, about 10 pM to about 200 nM, about 10 pM to about 150 nM, about 10 pM to about 100 nM, about 10 pM to about 95 nM, about 10 pM to about 90 nM, about 10 pM to about 85 nM, about 10 pM to about 80 nM, about 10 pM to about 75 nM, about 10 pM to about 70 nM, about 10 pM to about 75 nM, about 10 pM to about 70 nM, about 10 pM to about 65 nM, about 10 pM to about 60 nM, about 10 pM to about 55 nM, about 10 pM to about 50 nM, about 10 pM to about 45 nM, about 10 pM to about 40 nM, about 10 pM to about 35 nM, about 10 pM to about 30 nM, about 10 pM to about 25 nM, about 10 pM to about 20 nM, about 10 pM to about 15 nM, about 10 pM to about 10 nM, about 10 pM to about 5 nM, about 10 pM to about 2 nM, about 10 pM to about 1 nM, about 10 pM to about 950 pM, about 10 pM to about 900 pM, about 10 pM to about 850 pM, about 10 pM to about 800 pM, about 10 pM to about 750 pM, about 10 pM to about 700 pM, about 10 pM to about 650 pM, about 10 pM to about 600 pM, about 10 pM to about 550 pM, about 10 pM to about 500 pM, about 10 pM to about 450 pM, about 10 pM to about 400 pM, about 10 pM to about 350 pM, about 10 pM to about 300 pM, about 10 pM to about 250 pM, about 10 pM to about 200 pM, about 10 pM to about 150 pM, about 10 pM to about 100 pM, about 10 pM to about 95 pM, about 10 pM to about 90 pM, about 10 pM to about 85 pM, about 10 pM to about 80 pM, about 10 pM to about 75 pM, about 10 pM to about 70 pM, about 10 pM to about 75 pM, about 10 pM to about 70 pM, about 10 pM to about 65 pM, about 10 pM to about 60 pM, about 10 pM to about 55 pM, about 10 pM to about 50 pM, about 10 pM to about 45 pM, about 10 pM to about 40 pM, about 10 pM to about 35 pM, about 10 pM to about 30 pM, about 10 pM to about 25 pM, about 10 pM to about 20 pM, about 10 pM to about 15 pM, about 20 pM to about 100 µM, about 20 pM to about 95 µM, about 20 pM to about 90 µM, about 20 pM to about 85 µM, about 20 pM to about 80 µM, about 20 pM to about 75 µM, about 20 pM to about 70 µM, about 20 pM to about 65 µM, about 20 pM to about 60 µM, about 20 pM to about 55 µM, about 20 pM to about 50 µM, about 20 pM to about 45 µM, about 20 pM to about 40 µM, about 20 pM to about 45 µM, about 20 pM to about 40 µM, about 20 pM to about 35 µM, about 20 pM to about 30 µM, about 20 pM to about 25 µM, about 20 pM to about 20 µM, about 20 pM to about 15 µM, about 20 pM to about 10 µM, about 20 pM to about 5 µM, about 20 pM to about 2 µM, about 20 pM to about 1 µM, about 20 pM to about 950 nM, about 20 pM to about 900 nM, about 20 pM to about 850 nM, about 20 pM to about 800 nM, about 20 pM to about 750 nM, about 20 pM to about 700 nM, about 20 pM to about 650 nM, about 20 pM to about 600 nM, about 20 pM to about 550 nM, about 20 pM to about 500 nM, about 20 pM to about 450 nM, about 20 pM to about 400 nM, about 20 pM to about 350 nM, about 20 pM to about 300 nM, about 20 pM to about 250 nM, about 20 pM to about 200 nM, about 20 pM to about 150 nM, about 20 pM to about 100 nM, about 20 pM to about 95 nM, about 20 pM to about 90 nM, about 20 pM to about 85 nM, about 20 pM to about 80 nM, about 20 pM to about 75 nM, about 20 pM to about 70 nM, about 20 pM to about 75 nM, about 20 pM to about 70 nM, about 20 pM to about 65 nM, about 20 pM to about 60 nM, about 20 pM to about 55 nM, about 20 pM to about 50 nM, about 20 pM to about 45 nM, about 20 pM to about 40 nM, about 20 pM to about 35 nM, about 20 pM to about 30 nM, about 20 pM to about 25 nM, about 20 pM to about 20 nM, about 20 pM to about 15 nM, about 20 pM to about 10 nM, about 20 pM to about 5 nM, about 20 pM to about 2 nM, about 20 pM to about 1 nM, about 20 pM to about 950 pM, about 20 pM to about 900 pM, about 20 pM to about 850 pM, about 20 pM to about 800 pM, about 20 pM to about 750 pM, about 20 pM to about 700 pM, about 20 pM to about 650 pM, about 20 pM to about 600 pM, about 20 pM to about 550 pM, about 20 pM to about 500 pM, about 20 pM to about 450 pM, about 20 pM to about 400 pM, about 20 pM to about 350 pM, about 20 pM to about 300 pM, about 20 pM to about 250 pM, about 20 pM to about 200 pM, about 20 pM to about 150 pM, about 20 pM to about 100 pM, about 20 pM to about 95 pM, about 20 pM to about 90 pM, about 20 pM to about 85 pM, about 20 pM to about 80 pM, about 20 pM to about 75 pM, about 20 pM to about 70 pM, about 20 pM to about 75 pM, about 20 pM to about 70 pM, about 20 pM to about 65 pM, about 20 pM to about 60 pM, about 20 pM to about 55 pM, about 20 pM to about 50 pM, about 20 pM to about 45 pM, about 20 pM to about 40 pM, about 20 pM to about 35 pM, about 20 pM to about 30 pM, about 20 pM to about 25 pM, about 50 pM to about 100 µM, about 50 pM to about 95 µM, about 50 pM to about 90 µM, about 50 pM to about 85 µM, about 50 pM to about 80 µM, about 50 pM to about 75 µM, about 50 pM to about 70 µM, about 50 pM to about 65 µM, about 50 pM to about 60 µM, about 50 pM to about 55 µM, about 50 pM to about 50 µM, about 50 pM to about 45 µM, about 50 pM to about 40 µM, about 50 pM to about 45 µM, about 50 pM to about 40 µM, about 50 pM to about 35 µM, about 50 pM to about 30 µM, about 50 pM to about 25 µM, about 50 pM to about 20 µM, about 50 pM to about 15 µM, about 50 pM to about 10 µM, about 50 pM to about 5 µM, about 50 pM to about 2 µM, about 50 pM to about 1 µM, about 50 pM to about 950 nM, about 50 pM to about 900 nM, about 50 pM to about 850 nM, about 50 pM to about 800 nM, about 50 pM to about 750 nM, about 50 pM to about 700 nM, about 50 pM to about 650 nM, about 50 pM to about 600 nM, about 50 pM to about 550 nM, about 50 pM to about 500 nM, about 50 pM to about 450 nM, about 50 pM to about 400 nM, about 50 pM to about 350 nM, about 50 pM to about 300 nM, about 50 pM to about 250 nM, about 50 pM to about 200 nM, about 50 pM to about 150 nM, about 50 pM to about 100 nM, about 50 pM to about 95 nM, about 50 pM to about 90 nM, about 50 pM to about 85 nM, about 50 pM to about 80 nM, about 50 pM to about 75 nM, about 50 pM to about 70 nM, about 50 pM to about 75 nM, about 50 pM to about 70 nM, about 50 pM to about 65 nM, about 50 pM to about 60 nM, about 50 pM to about 55 nM, about 50 pM to about 50 nM, about 50 pM to about 45 nM, about 50 pM to about 40 nM, about 50 pM to about 35 nM, about 50 pM to about 30 nM, about 50 pM to about 25 nM, about 50 pM to about 20 nM, about 50 pM to about 15 nM, about 50 pM to about 10 nM, about 50 pM to about 5 nM, about 50 pM to about 2 nM, about 50 pM to about 1 nM, about 50 pM to about 950 pM, about 50 pM to about 900 pM, about 50 pM to about 850 pM, about 50 pM to about 800 pM, about 50 pM to about 750 pM, about 50 pM to about 700 pM, about 50 pM to about 650 pM, about 50 pM to about 600 pM, about 50 pM to about 550 pM, about 50 pM to about 500 pM, about 50 pM to about 450 pM, about 50 pM to about 400 pM, about 50 pM to about 350 pM, about 50 pM to about 300 pM, about 50 pM to about 250 pM, about 50 pM to about 200 pM, about 50 pM to about 150 pM, about 50 pM to about 100 pM, about 50 pM to about 95 pM, about 50 pM to about 90 pM, about 50 pM to about 85 pM, about 50 pM to about 80 pM, about 50 pM to about 75 pM, about 50 pM to about 70 pM, about 50 pM to about 75 pM, about 50 pM to about 70 pM, about 50 pM to about 65 pM, about 50 pM to about 60 pM, about 50 pM to about 55 pM, about 100 pM to about 100 µM, about 100 pM to about 95 µM, about 100 pM to about 90 µM, about 100 pM to about 85 µM, about 100 pM to about 80 µM, about 100 pM to about 75 µM, about 100 pM to about 70 µM, about 100 pM to about 65 µM, about 100 pM to about 60 µM, about 100 pM to about 55 µM, about 100 pM to about 50 µM, about 100 pM to about 45 µM, about 100 pM to about 40 µM, about 100 pM to about 45 µM, about 100 pM to about 40 µM, about 100 pM to about 35 µM, about 100 pM to about 30 µM, about 100 pM to about 25 µM, about 100 pM to about 20 µM, about 100 pM to about 15 µM, about 100 pM to about 10 µM, about 100 pM to about 5 µM, about 100 pM to about 2 µM, about 100 pM to about 1 µM, about 100 pM to about 950 nM, about 100 pM to about 900 nM, about 100 pM to about 850 nM, about 100 pM to about 800 nM, about 100 pM to about 750 nM, about 100 pM to about 700 nM, about 100 pM to about 650 nM, about 100 pM to about 600 nM, about 100 pM to about 550 nM, about 100 pM to about 500 nM, about 100 pM to about 450 nM, about 100 pM to about 400 nM, about 100 pM to about 350 nM, about 100 pM to about 300 nM, about 100 pM to about 250 nM, about 100 pM to about 200 nM, about 100 pM to about 150 nM, about 100 pM to about 100 nM, about 100 pM to about 95 nM, about 100 pM to about 90 nM, about 100 pM to about 85 nM, about 100 pM to about 80 nM, about 100 pM to about 75 nM, about 100 pM to about 70 nM, about 100 pM to about 75 nM, about 100 pM to about 70 nM, about 100 pM to about 65 nM, about 100 pM to about 60 nM, about 100 pM to about 55 nM, about 100 pM to about 50 nM, about 100 pM to about 45 nM, about 100 pM to about 40 nM, about 100 pM to about 35 nM, about 100 pM to about 30 nM, about 100 pM to about 25 nM, about 100 pM to about 20 nM, about 100 pM to about 15 nM, about 100 pM to about 10 nM, about 100 pM to about 5 nM, about 100 pM to about 2 nM, about 100 pM to about 1 nM, about 100 pM to about 950 pM, about 100 pM to about 900 pM, about 100 pM to about 850 pM, about 100 pM to about 800 pM, about 100 pM to about 750 pM, about 100 pM to about 700 pM, about 100 pM to about 650 pM, about 100 pM to about 600 pM, about 100 pM to about 550 pM, about 100 pM to about 500 pM, about 100 pM to about 450 pM, about 100 pM to about 400 pM, about 100 pM to about 350 pM, about 100 pM to about 300 pM, about 100 pM to about 250 pM, about 100 pM to about 200 pM, about 100 pM to about 150 pM, about 200 pM to about 100 µM, about 200 pM to about 95 µM, about 200 pM to about 90 µM, about 200 pM to about 85 µM, about 200 pM to about 80 µM, about 200 pM to about 75 µM, about 200 pM to about 70 µM, about 200 pM to about 65 µM, about 200 pM to about 60 µM, about 200 pM to about 55 µM, about 200 pM to about 50 µM, about 200 pM to about 45 µM, about 200 pM to about 40 µM, about 200 pM to about 45 µM, about 200 pM to about 40 µM, about 200 pM to about 35 µM, about 200 pM to about 30 µM, about 200 pM to about 25 µM, about 200 pM to about 20 µM, about 200 pM to about 15 µM, about 200 pM to about 10 µM, about 200 pM to about 5 µM, about 200 pM to about 2 µM, about 200 pM to about 1 µM, about 200 pM to about 950 nM, about 200 pM to about 900 nM, about 200 pM to about 850 nM, about 200 pM to about 800 nM, about 200 pM to about 750 nM, about 200 pM to about 700 nM, about 200 pM to about 650 nM, about 200 pM to about 600 nM, about 200 pM to about 550 nM, about 200 pM to about 500 nM, about 200 pM to about 450 nM, about 200 pM to about 400 nM, about 200 pM to about 350 nM, about 200 pM to about 300 nM, about 200 pM to about 250 nM, about 200 pM to about 200 nM, about 200 pM to about 150 nM, about 200 pM to about 100 nM, about 200 pM to about 95 nM, about 200 pM to about 90 nM, about 200 pM to about 85 nM, about 200 pM to about 80 nM, about 200 pM to about 75 nM, about 200 pM to about 70 nM, about 200 pM to about 75 nM, about 200 pM to about 70 nM, about 200 pM to about 65 nM, about 200 pM to about 60 nM, about 200 pM to about 55 nM, about 200 pM to about 50 nM, about 200 pM to about 45 nM, about 200 pM to about 40 nM, about 200 pM to about 35 nM, about 200 pM to about 30 nM, about 200 pM to about 25 nM, about 200 pM to about 20 nM, about 200 pM to about 15 nM, about 200 pM to about 10 nM, about 200 pM to about 5 nM, about 200 pM to about 2 nM, about 200 pM to about 1 nM, about 200 pM to about 950 pM, about 200 pM to about 900 pM, about 200 pM to about 850 pM, about 200 pM to about 800 pM, about 200 pM to about 750 pM, about 200 pM to about 700 pM, about 200 pM to about 650 pM, about 200 pM to about 600 pM, about 200 pM to about 550 pM, about 200 pM to about 500 pM, about 200 pM to about 450 pM, about 200 pM to about 400 pM, about 200 pM to about 350 pM, about 200 pM to about 300 pM, about 200 pM to about 250 pM, about 300 pM to about 100 µM, about 300 pM to about 95 µM, about 300 pM to about 90 µM, about 300 pM to about 85 µM, about 300 pM to about 80 µM, about 300 pM to about 75 µM, about 300 pM to about 70 µM, about 300 pM to about 65 µM, about 300 pM to about 60 µM, about 300 pM to about 55 µM, about 300 pM to about 50 µM, about 300 pM to about 45 µM, about 300 pM to about 40 µM, about 300 pM to about 45 µM, about 300 pM to about 40 µM, about 300 pM to about 35 µM, about 300 pM to about 30 µM, about 300 pM to about 25 µM, about 300 pM to about 20 µM, about 300 pM to about 15 µM, about 300 pM to about 10 µM, about 300 pM to about 5 µM, about 300 pM to about 2 µM, about 300 pM to about 1 µM, about 300 pM to about 950 nM, about 300 pM to about 900 nM, about 300 pM to about 850 nM, about 300 pM to about 800 nM, about 300 pM to about 750 nM, about 300 pM to about 700 nM, about 300 pM to about 650 nM, about 300 pM to about 600 nM, about 300 pM to about 550 nM, about 300 pM to about 500 nM, about 300 pM to about 450 nM, about 300 pM to about 400 nM, about 300 pM to about 350 nM, about 300 pM to about 300 nM, about 300 pM to about 250 nM, about 300 pM to about 200 nM, about 300 pM to about 150 nM, about 300 pM to about 100 nM, about 300 pM to about 95 nM, about 300 pM to about 90 nM, about 300 pM to about 85 nM, about 300 pM to about 80 nM, about 300 pM to about 75 nM, about 300 pM to about 70 nM, about 300 pM to about 75 nM, about 300 pM to about 70 nM, about 300 pM to about 65 nM, about 300 pM to about 60 nM, about 300 pM to about 55 nM, about 300 pM to about 50 nM, about 300 pM to about 45 nM, about 300 pM to about 40 nM, about 300 pM to about 35 nM, about 300 pM to about 30 nM, about 300 pM to about 25 nM, about 300 pM to about 20 nM, about 300 pM to about 15 nM, about 300 pM to about 10 nM, about 300 pM to about 5 nM, about 300 pM to about 2 nM, about 300 pM to about 1 nM, about 300 pM to about 950 pM, about 300 pM to about 900 pM, about 300 pM to about 850 pM, about 300 pM to about 800 pM, about 300 pM to about 750 pM, about 300 pM to about 700 pM, about 300 pM to about 650 pM, about 300 pM to about 600 pM, about 300 pM to about 550 pM, about 300 pM to about 500 pM, about 300 pM to about 450 pM, about 300 pM to about 400 pM, about 300 pM to about 350 pM, about 400 pM to about 100 µM, about 400 pM to about 95 µM, about 400 pM to about 90 µM, about 400 pM to about 85 µM, about 400 pM to about 80 µM, about 400 pM to about 75 µM, about 400 pM to about 70 µM, about 400 pM to about 65 µM, about 400 pM to about 60 µM, about 400 pM to about 55 µM, about 400 pM to about 50 µM, about 400 pM to about 45 µM, about 400 pM to about 40 µM, about 400 pM to about 45 µM, about 400 pM to about 40 µM, about 400 pM to about 35 µM, about 400 pM to about 30 µM, about 400 pM to about 25 µM, about 400 pM to about 20 µM, about 400 pM to about 15 µM, about 400 pM to about 10 µM, about 400 pM to about 5 µM, about 400 pM to about 2 µM, about 400 pM to about 1 µM, about 400 pM to about 950 nM, about 400 pM to about 900 nM, about 400 pM to about 850 nM, about 400 pM to about 800 nM, about 400 pM to about 750 nM, about 400 pM to about 700 nM, about 400 pM to about 650 nM, about 400 pM to about 600 nM, about 400 pM to about 550 nM, about 400 pM to about 500 nM, about 400 pM to about 450 nM, about 400 pM to about 400 nM, about 400 pM to about 350 nM, about 400 pM to about 300 nM, about 400 pM to about 250 nM, about 400 pM to about 200 nM, about 400 pM to about 150 nM, about 400 pM to about 100 nM, about 400 pM to about 95 nM, about 400 pM to about 90 nM, about 400 pM to about 85 nM, about 400 pM to about 80 nM, about 400 pM to about 75 nM, about 400 pM to about 70 nM, about 400 pM to about 75 nM, about 400 pM to about 70 nM, about 400 pM to about 65 nM, about 400 pM to about 60 nM, about 400 pM to about 55 nM, about 400 pM to about 50 nM, about 400 pM to about 45 nM, about 400 pM to about 40 nM, about 400 pM to about 35 nM, about 400 pM to about 30 nM, about 400 pM to about 25 nM, about 400 pM to about 20 nM, about 400 pM to about 15 nM, about 400 pM to about 10 nM, about 400 pM to about 5 nM, about 400 pM to about 2 nM, about 400 pM to about 1 nM, about 400 pM to about 950 pM, about 400 pM to about 900 pM, about 400 pM to about 850 pM, about 400 pM to about 800 pM, about 400 pM to about 750 pM, about 400 pM to about 700 pM, about 400 pM to about 650 pM, about 400 pM to about 600 pM, about 400 pM to about 550 pM, about 400 pM to about 500 pM, about 400 pM to about 450 pM, about 500 pM to about 100 µM, about 500 pM to about 95 µM, about 500 pM to about 90 µM, about 500 pM to about 85 µM, about 500 pM to about 80 µM, about 500 pM to about 75 µM, about 500 pM to about 70 µM, about 500 pM to about 65 µM, about 500 pM to about 60 µM, about 500 pM to about 55 µM, about 500 pM to about 50 µM, about 500 pM to about 45 µM, about 500 pM to about 40 µM, about 500 pM to about 45 µM, about 500 pM to about 40 µM, about 500 pM to about 35 µM, about 500 pM to about 30 µM, about 500 pM to about 25 µM, about 500 pM to about 20 µM, about 500 pM to about 15 µM, about 500 pM to about 10 µM, about 500 pM to about 5 µM, about 500 pM to about 2 µM, about 500 pM to about 1 µM, about 500 pM to about 950 nM, about 500 pM to about 900 nM, about 500 pM to about 850 nM, about 500 pM to about 800 nM, about 500 pM to about 750 nM, about 500 pM to about 700 nM, about 500 pM to about 650 nM, about 500 pM to about 600 nM, about 500 pM to about 550 nM, about 500 pM to about 500 nM, about 500 pM to about 450 nM, about 500 pM to about 400 nM, about 500 pM to about 350 nM, about 500 pM to about 300 nM, about 500 pM to about 250 nM, about 500 pM to about 200 nM, about 500 pM to about 150 nM, about 500 pM to about 100 nM, about 500 pM to about 95 nM, about 500 pM to about 90 nM, about 500 pM to about 85 nM, about 500 pM to about 80 nM, about 500 pM to about 75 nM, about 500 pM to about 70 nM, about 500 pM to about 75 nM, about 500 pM to about 70 nM, about 500 pM to about 65 nM, about 500 pM to about 60 nM, about 500 pM to about 55 nM, about 500 pM to about 50 nM, about 500 pM to about 45 nM, about 500 pM to about 40 nM, about 500 pM to about 35 nM, about 500 pM to about 30 nM, about 500 pM to about 25 nM, about 500 pM to about 20 nM, about 500 pM to about 15 nM, about 500 pM to about 10 nM, about 500 pM to about 5 nM, about 500 pM to about 2 nM, about 500 pM to about 1 nM, about 500 pM to about 950 pM, about 500 pM to about 900 pM, about 500 pM to about 850 pM, about 500 pM to about 800 pM, about 500 pM to about 750 pM, about 500 pM to about 700 pM, about 500 pM to about 650 pM, about 500 pM to about 600 pM, about 500 pM to about 550 pM, about 600 pM to about 100 µM, about 600 pM to about 95 µM, about 600 pM to about 90 µM, about 600 pM to about 85 µM, about 600 pM to about 80 µM, about 600 pM to about 75 µM, about 600 pM to about 70 µM, about 600 pM to about 65 µM, about 600 pM to about 60 µM, about 600 pM to about 55 µM, about 600 pM to about 50 µM, about 600 pM to about 45 µM, about 600 pM to about 40 µM, about 600 pM to about 45 µM, about 600 pM to about 40 µM, about 600 pM to about 35 µM, about 600 pM to about 30 µM, about 600 pM to about 25 µM, about 600 pM to about 20 µM, about 600 pM to about 15 µM, about 600 pM to about 10 µM, about 600 pM to about 5 µM, about 600 pM to about 2 µM, about 600 pM to about 1 µM, about 600 pM to about 950 nM, about 600 pM to about 900 nM, about 600 pM to about 850 nM, about 600 pM to about 800 nM, about 600 pM to about 750 nM, about 600 pM to about 700 nM, about 600 pM to about 650 nM, about 600 pM to about 600 nM, about 600 pM to about 550 nM, about 600 pM to about 500 nM, about 600 pM to about 450 nM, about 600 pM to about 400 nM, about 600 pM to about 350 nM, about 600 pM to about 300 nM, about 600 pM to about 250 nM, about 600 pM to about 200 nM, about 600 pM to about 150 nM, about 600 pM to about 100 nM, about 600 pM to about 95 nM, about 600 pM to about 90 nM, about 600 pM to about 85 nM, about 600 pM to about 80 nM, about 600 pM to about 75 nM, about 600 pM to about 70 nM, about 600 pM to about 75 nM, about 600 pM to about 70 nM, about 600 pM to about 65 nM, about 600 pM to about 60 nM, about 600 pM to about 55 nM, about 600 pM to about 50 nM, about 600 pM to about 45 nM, about 600 pM to about 40 nM, about 600 pM to about 35 nM, about 600 pM to about 30 nM, about 600 pM to about 25 nM, about 600 pM to about 20 nM, about 600 pM to about 15 nM, about 600 pM to about 10 nM, about 600 pM to about 5 nM, about 600 pM to about 2 nM, about 600 pM to about 1 nM, about 600 pM to about 950 pM, about 600 pM to about 900 pM, about 600 pM to about 850 pM, about 600 pM to about 800 pM, about 600 pM to about 750 pM, about 600 pM to about 700 pM, about 600 pM to about 650 pM, about 700 pM to about 100 µM, about 700 pM to about 95 µM, about 700 pM to about 90 µM, about 700 pM to about 85 µM, about 700 pM to about 80 µM, about 700 pM to about 75 µM, about 700 pM to about 70 µM, about 700 pM to about 65 µM, about 700 pM to about 60 µM, about 700 pM to about 55 µM, about 700 pM to about 50 µM, about 700 pM to about 45 µM, about 700 pM to about 40 µM, about 700 pM to about 45 µM, about 700 pM to about 40 µM, about 700 pM to about 35 µM, about 700 pM to about 30 µM, about 700 pM to about 25 µM, about 700 pM to about 20 µM, about 700 pM to about 15 µM, about 700 pM to about 10 µM, about 700 pM to about 5 µM, about 700 pM to about 2 µM, about 700 pM to about 1 µM, about 700 pM to about 950 nM, about 700 pM to about 900 nM, about 700 pM to about 850 nM, about 700 pM to about 800 nM, about 700 pM to about 750 nM, about 700 pM to about 700 nM, about 700 pM to about 650 nM, about 700 pM to about 600 nM, about 700 pM to about 550 nM, about 700 pM to about 500 nM, about 700 pM to about 450 nM, about 700 pM to about 400 nM, about 700 pM to about 350 nM, about 700 pM to about 300 nM, about 700 pM to about 250 nM, about 700 pM to about 200 nM, about 700 pM to about 150 nM, about 700 pM to about 100 nM, about 700 pM to about 95 nM, about 700 pM to about 90 nM, about 700 pM to about 85 nM, about 700 pM to about 80 nM, about 700 pM to about 75 nM, about 700 pM to about 70 nM, about 700 pM to about 75 nM, about 700 pM to about 70 nM, about 700 pM to about 65 nM, about 700 pM to about 60 nM, about 700 pM to about 55 nM, about 700 pM to about 50 nM, about 700 pM to about 45 nM, about 700 pM to about 40 nM, about 700 pM to about 35 nM, about 700 pM to about 30 nM, about 700 pM to about 25 nM, about 700 pM to about 20 nM, about 700 pM to about 15 nM, about 700 pM to about 10 nM, about 700 pM to about 5 nM, about 700 pM to about 2 nM, about 700 pM to about 1 nM, about 700 pM to about 950 pM, about 700 pM to about 900 pM, about 700 pM to about 850 pM, about 700 pM to about 800 pM, about 700 pM to about 750 pM, about 800 pM to about 100 µM, about 800 pM to about 95 µM, about 800 pM to about 90 µM, about 800 pM to about 85 µM, about 800 pM to about 80 µM, about 800 pM to about 75 µM, about 800 pM to about 70 µM, about 800 pM to about 65 µM, about 800 pM to about 60 µM, about 800 pM to about 55 µM, about 800 pM to about 50 µM, about 800 pM to about 45 µM, about 800 pM to about 40 µM, about 800 pM to about 45 µM, about 800 pM to about 40 µM, about 800 pM to about 35 µM, about 800 pM to about 30 µM, about 800 pM to about 25 µM, about 800 pM to about 20 µM, about 800 pM to about 15 µM, about 800 pM to about 10 µM, about 800 pM to about 5 µM, about 800 pM to about 2 µM, about 800 pM to about 1 µM, about 800 pM to about 950 nM, about 800 pM to about 900 nM, about 800 pM to about 850 nM, about 800 pM to about 800 nM, about 800 pM to about 750 nM, about 800 pM to about 700 nM, about 800 pM to about 650 nM, about 800 pM to about 600 nM, about 800 pM to about 550 nM, about 800 pM to about 500 nM, about 800 pM to about 450 nM, about 800 pM to about 400 nM, about 800 pM to about 350 nM, about 800 pM to about 300 nM, about 800 pM to about 250 nM, about 800 pM to about 200 nM, about 800 pM to about 150 nM, about 800 pM to about 100 nM, about 800 pM to about 95 nM, about 800 pM to about 90 nM, about 800 pM to about 85 nM, about 800 pM to about 80 nM, about 800 pM to about 75 nM, about 800 pM to about 70 nM, about 800 pM to about 75 nM, about 800 pM to about 70 nM, about 800 pM to about 65 nM, about 800 pM to about 60 nM, about 800 pM to about 55 nM, about 800 pM to about 50 nM, about 800 pM to about 45 nM, about 800 pM to about 40 nM, about 800 pM to about 35 nM, about 800 pM to about 30 nM, about 800 pM to about 25 nM, about 800 pM to about 20 nM, about 800 pM to about 15 nM, about 800 pM to about 10 nM, about 800 pM to about 5 nM, about 800 pM to about 2 nM, about 800 pM to about 1 nM, about 800 pM to about 950 pM, about 800 pM to about 900 pM, about 800 pM to about 850 pM, about 900 pM to about 100 µM, about 900 pM to about 95 µM, about 900 pM to about 90 µM, about 900 pM to about 85 µM, about 900 pM to about 80 µM, about 900 pM to about 75 µM, about 900 pM to about 70 µM, about 900 pM to about 65 µM, about 900 pM to about 60 µM, about 900 pM to about 55 µM, about 900 pM to about 50 µM, about 900 pM to about 45 µM, about 900 pM to about 40 µM, about 900 pM to about 45 µM, about 900 pM to about 40 µM, about 900 pM to about 35 µM, about 900 pM to about 30 µM, about 900 pM to about 25 µM, about 900 pM to about 20 µM, about 900 pM to about 15 µM, about 900 pM to about 10 µM, about 900 pM to about 5 µM, about 900 pM to about 2 µM, about 900 pM to about 1 µM, about 900 pM to about 950 nM, about 900 pM to about 900 nM, about 900 pM to about 850 nM, about 900 pM to about 800 nM, about 900 pM to about 750 nM, about 900 pM to about 700 nM, about 900 pM to about 650 nM, about 900 pM to about 600 nM, about 900 pM to about 550 nM, about 900 pM to about 500 nM, about 900 pM to about 450 nM, about 900 pM to about 400 nM, about 900 pM to about 350 nM, about 900 pM to about 300 nM, about 900 pM to about 250 nM, about 900 pM to about 200 nM, about 900 pM to about 150 nM, about 900 pM to about 100 nM, about 900 pM to about 95 nM, about 900 pM to about 90 nM, about 900 pM to about 85 nM, about 900 pM to about 80 nM, about 900 pM to about 75 nM, about 900 pM to about 70 nM, about 900 pM to about 75 nM, about 900 pM to about 70 nM, about 900 pM to about 65 nM, about 900 pM to about 60 nM, about 900 pM to about 55 nM, about 900 pM to about 50 nM, about 900 pM to about 45 nM, about 900 pM to about 40 nM, about 900 pM to about 35 nM, about 900 pM to about 30 nM, about 900 pM to about 25 nM, about 900 pM to about 20 nM, about 900 pM to about 15 nM, about 900 pM to about 10 nM, about 900 pM to about 5 nM, about 900 pM to about 2 nM, about 900 pM to about 1 nM, about 900 pM to about 950 pM, about 1 nM to about 100 µM, about 1 nM to about 95 µM, about 1 nM to about 90 µM, about 1 nM to about 85 µM, about 1 nM to about 80 µM, about 1 nM to about 75 µM, about 1 nM to about 70 µM, about 1 nM to about 65 µM, about 1 nM to about 60 µM, about 1 nM to about 55 µM, about 1 nM to about 50 µM, about 1 nM to about 45 µM, about 1 nM to about 40 µM, about 1 nM to about 45 µM, about 1 nM to about 40 µM, about 1 nM to about 35 µM, about 1 nM to about 30 µM, about 1 nM to about 25 µM, about 1 nM to about 20 µM, about 1 nM to about 15 µM, about 1 nM to about 10 µM, about 1 nM to about 5 µM, about 1 nM to about 2 µM, about 1 nM to about 1 µM, about 1 nM to about 950 nM, about 1 nM to about 900 nM, about 1 nM to about 850 nM, about 1 nM to about 800 nM, about 1 nM to about 750 nM, about 1 nM to about 700 nM, about 1 nM to about 650 nM, about 1 nM to about 600 nM, about 1 nM to about 550 nM, about 1 nM to about 500 nM, about 1 nM to about 450 nM, about 1 nM to about 400 nM, about 1 nM to about 350 nM, about 1 nM to about 300 nM, about 1 nM to about 250 nM, about 1 nM to about 200 nM, about 1 nM to about 150 nM, about 1 nM to about 100 nM, about 1 nM to about 95 nM, about 1 nM to about 90 nM, about 1 nM to about 85 nM, about 1 nM to about 80 nM, about 1 nM to about 75 nM, about 1 nM to about 70 nM, about 1 nM to about 75 nM, about 1 nM to about 70 nM, about 1 nM to about 65 nM, about 1 nM to about 60 nM, about 1 nM to about 55 nM, about 1 nM to about 50 nM, about 1 nM to about 45 nM, about 1 nM to about 40 nM, about 1 nM to about 35 nM, about 1 nM to about 30 nM, about 1 nM to about 25 nM, about 1 nM to about 20 nM, about 1 nM to about 15 nM, about 1 nM to about 10 nM, about 1 nM to about 5 nM, about 1 nM to about 2 nM, about 2 nM to about 100 µM, about 2 nM to about 95 µM, about 2 nM to about 90 µM, about 2 nM to about 85 µM, about 2 nM to about 80 µM, about 2 nM to about 75 µM, about 2 nM to about 70 µM, about 2 nM to about 65 µM, about 2 nM to about 60 µM, about 2 nM to about 55 µM, about 2 nM to about 50 µM, about 2 nM to about 45 µM, about 2 nM to about 40 µM, about 2 nM to about 45 µM, about 2 nM to about 40 µM, about 2 nM to about 35 µM, about 2 nM to about 30 µM, about 2 nM to about 25 µM, about 2 nM to about 20 µM, about 2 nM to about 15 µM, about 2 nM to about 10 µM, about 2 nM to about 5 µM, about 2 nM to about 2 µM, about 2 nM to about 1 µM, about 2 nM to about 950 nM, about 2 nM to about 900 nM, about 2 nM to about 850 nM, about 2 nM to about 800 nM, about 2 nM to about 750 nM, about 2 nM to about 700 nM, about 2 nM to about 650 nM, about 2 nM to about 600 nM, about 2 nM to about 550 nM, about 2 nM to about 500 nM, about 2 nM to about 450 nM, about 2 nM to about 400 nM, about 2 nM to about 350 nM, about 2 nM to about 300 nM, about 2 nM to about 250 nM, about 2 nM to about 200 nM, about 2 nM to about 150 nM, about 2 nM to about 100 nM, about 2 nM to about 95 nM, about 2 nM to about 90 nM, about 2 nM to about 85 nM, about 2 nM to about 80 nM, about 2 nM to about 75 nM, about 2 nM to about 70 nM, about 2 nM to about 75 nM, about 2 nM to about 70 nM, about 2 nM to about 65 nM, about 2 nM to about 60 nM, about 2 nM to about 55 nM, about 2 nM to about 50 nM, about 2 nM to about 45 nM, about 2 nM to about 40 nM, about 2 nM to about 35 nM, about 2 nM to about 30 nM, about 2 nM to about 25 nM, about 2 nM to about 20 nM, about 2 nM to about 15 nM, about 2 nM to about 10 nM, about 2 nM to about 5 nM, about 5 nM to about 100 µM, about 5 nM to about 95 µM, about 5 nM to about 90 µM, about 5 nM to about 85 µM, about 5 nM to about 80 µM, about 5 nM to about 75 µM, about 5 nM to about 70 µM, about 5 nM to about 65 µM, about 5 nM to about 60 µM, about 5 nM to about 55 µM, about 5 nM to about 50 µM, about 5 nM to about 45 µM, about 5 nM to about 40 µM, about 5 nM to about 45 µM, about 5 nM to about 40 µM, about 5 nM to about 35 µM, about 5 nM to about 30 µM, about 5 nM to about 25 µM, about 5 nM to about 20 µM, about 5 nM to about 15 µM, about 5 nM to about 10 µM, about 5 nM to about 5 µM, about 5 nM to about 2 µM, about 5 nM to about 1 µM, about 5 nM to about 950 nM, about 5 nM to about 900 nM, about 5 nM to about 850 nM, about 5 nM to about 800 nM, about 5 nM to about 750 nM, about 5 nM to about 700 nM, about 5 nM to about 650 nM, about 5 nM to about 600 nM, about 5 nM to about 550 nM, about 5 nM to about 500 nM, about 5 nM to about 450 nM, about 5 nM to about 400 nM, about 5 nM to about 350 nM, about 5 nM to about 300 nM, about 5 nM to about 250 nM, about 5 nM to about 200 nM, about 5 nM to about 150 nM, about 5 nM to about 100 nM, about 5 nM to about 95 nM, about 5 nM to about 90 nM, about 5 nM to about 85 nM, about 5 nM to about 80 nM, about 5 nM to about 75 nM, about 5 nM to about 70 nM, about 5 nM to about 75 nM, about 5 nM to about 70 nM, about 5 nM to about 65 nM, about 5 nM to about 60 nM, about 5 nM to about 55 nM, about 5 nM to about 50 nM, about 5 nM to about 45 nM, about 5 nM to about 40 nM, about 5 nM to about 35 nM, about 5 nM to about 30 nM, about 5 nM to about 25 nM, about 5 nM to about 20 nM, about 5 nM to about 15 nM, about 5 nM to about 10 nM, about 10 nM to about 100 µM, about 10 nM to about 95 µM, about 10 nM to about 90 µM, about 10 nM to about 85 µM, about 10 nM to about 80 µM, about 10 nM to about 75 µM, about 10 nM to about 70 µM, about 10 nM to about 65 µM, about 10 nM to about 60 µM, about 10 nM to about 55 µM, about 10 nM to about 50 µM, about 10 nM to about 45 µM, about 10 nM to about 40 µM, about 10 nM to about 45 µM, about 10 nM to about 40 µM, about 10 nM to about 35 µM, about 10 nM to about 30 µM, about 10 nM to about 25 µM, about 10 nM to about 20 µM, about 10 nM to about 15 µM, about 10 nM to about 10 µM, about 10 nM to about 5 µM, about 10 nM to about 2 µM, about 10 nM to about 1 µM, about 10 nM to about 950 nM, about 10 nM to about 900 nM, about 10 nM to about 850 nM, about 10 nM to about 800 nM, about 10 nM to about 750 nM, about 10 nM to about 700 nM, about 10 nM to about 650 nM, about 10 nM to about 600 nM, about 10 nM to about 550 nM, about 10 nM to about 500 nM, about 10 nM to about 450 nM, about 10 nM to about 400 nM, about 10 nM to about 350 nM, about 10 nM to about 300 nM, about 10 nM to about 250 nM, about 10 nM to about 200 nM, about 10 nM to about 150 nM, about 10 nM to about 100 nM, about 10 nM to about 95 nM, about 10 nM to about 90 nM, about 10 nM to about 85 nM, about 10 nM to about 80 nM, about 10 nM to about 75 nM, about 10 nM to about 70 nM, about 10 nM to about 75 nM, about 10 nM to about 70 nM, about 10 nM to about 65 nM, about 10 nM to about 60 nM, about 10 nM to about 55 nM, about 10 nM to about 50 nM, about 10 nM to about 45 nM, about 10 nM to about 40 nM, about 10 nM to about 35 nM, about 10 nM to about 30 nM, about 10 nM to about 25 nM, about 10 nM to about 20 nM, about 10 nM to about 15 nM, about 20 nM to about 100 µM, about 20 nM to about 95 µM, about 20 nM to about 90 µM, about 20 nM to about 85 µM, about 20 nM to about 80 µM, about 20 nM to about 75 µM, about 20 nM to about 70 µM, about 20 nM to about 65 µM, about 20 nM to about 60 µM, about 20 nM to about 55 µM, about 20 nM to about 50 µM, about 20 nM to about 45 µM, about 20 nM to about 40 µM, about 20 nM to about 45 µM, about 20 nM to about 40 µM, about 20 nM to about 35 µM, about 20 nM to about 30 µM, about 20 nM to about 25 µM, about 20 nM to about 20 µM, about 20 nM to about 15 µM, about 20 nM to about 10 µM, about 20 nM to about 5 µM, about 20 nM to about 2 µM, about 20 nM to about 1 µM, about 20 nM to about 950 nM, about 20 nM to about 900 nM, about 20 nM to about 850 nM, about 20 nM to about 800 nM, about 20 nM to about 750 nM, about 20 nM to about 700 nM, about 20 nM to about 650 nM, about 20 nM to about 600 nM, about 20 nM to about 550 nM, about 20 nM to about 500 nM, about 20 nM to about 450 nM, about 20 nM to about 400 nM, about 20 nM to about 350 nM, about 20 nM to about 300 nM, about 20 nM to about 250 nM, about 20 nM to about 200 nM, about 20 nM to about 150 nM, about 20 nM to about 100 nM, about 20 nM to about 95 nM, about 20 nM to about 90 nM, about 20 nM to about 85 nM, about 20 nM to about 80 nM, about 20 nM to about 75 nM, about 20 nM to about 70 nM, about 20 nM to about 75 nM, about 20 nM to about 70 nM, about 20 nM to about 65 nM, about 20 nM to about 60 nM, about 20 nM to about 55 nM, about 20 nM to about 50 nM, about 20 nM to about 45 nM, about 20 nM to about 40 nM, about 20 nM to about 35 nM, about 20 nM to about 30 nM, about 20 nM to about 25 nM, about 50 nM to about 100 µM, about 50 nM to about 95 µM, about 50 nM to about 90 µM, about 50 nM to about 85 µM, about 50 nM to about 80 µM, about 50 nM to about 75 µM, about 50 nM to about 70 µM, about 50 nM to about 65 µM, about 50 nM to about 60 µM, about 50 nM to about 55 µM, about 50 nM to about 50 µM, about 50 nM to about 45 µM, about 50 nM to about 40 µM, about 50 nM to about 45 µM, about 50 nM to about 40 µM, about 50 nM to about 35 µM, about 50 nM to about 30 µM, about 50 nM to about 25 µM, about 50 nM to about 20 µM, about 50 nM to about 15 µM, about 50 nM to about 10 µM, about 50 nM to about 5 µM, about 50 nM to about 2 µM, about 50 nM to about 1 µM, about 50 nM to about 950 nM, about 50 nM to about 900 nM, about 50 nM to about 850 nM, about 50 nM to about 800 nM, about 50 nM to about 750 nM, about 50 nM to about 700 nM, about 50 nM to about 650 nM, about 50 nM to about 600 nM, about 50 nM to about 550 nM, about 50 nM to about 500 nM, about 50 nM to about 450 nM, about 50 nM to about 400 nM, about 50 nM to about 350 nM, about 50 nM to about 300 nM, about 50 nM to about 250 nM, about 50 nM to about 200 nM, about 50 nM to about 150 nM, about 50 nM to about 100 nM, about 50 nM to about 95 nM, about 50 nM to about 90 nM, about 50 nM to about 85 nM, about 50 nM to about 80 nM, about 50 nM to about 75 nM, about 50 nM to about 70 nM, about 50 nM to about 75 nM, about 50 nM to about 70 nM, about 50 nM to about 65 nM, about 50 nM to about 60 nM, about 50 nM to about 55 nM, about 100 nM to about 100 µM, about 100 nM to about 95 µM, about 100 nM to about 90 µM, about 100 nM to about 85 µM, about 100 nM to about 80 µM, about 100 nM to about 75 µM, about 100 nM to about 70 µM, about 100 nM to about 65 µM, about 100 nM to about 60 µM, about 100 nM to about 55 µM, about 100 nM to about 50 µM, about 100 nM to about 45 µM, about 100 nM to about 40 µM, about 100 nM to about 45 µM, about 100 nM to about 40 µM, about 100 nM to about 35 µM, about 100 nM to about 30 µM, about 100 nM to about 25 µM, about 100 nM to about 20 µM, about 100 nM to about 15 µM, about 100 nM to about 10 µM, about 100 nM to about 5 µM, about 100 nM to about 2 µM, about 100 nM to about 1 µM, about 100 nM to about 950 nM, about 100 nM to about 900 nM, about 100 nM to about 850 nM, about 100 nM to about 800 nM, about 100 nM to about 750 nM, about 100 nM to about 700 nM, about 100 nM to about 650 nM, about 100 nM to about 600 nM, about 100 nM to about 550 nM, about 100 nM to about 500 nM, about 100 nM to about 450 nM, about 100 nM to about 400 nM, about 100 nM to about 350 nM, about 100 nM to about 300 nM, about 100 nM to about 250 nM, about 100 nM to about 200 nM, about 100 nM to about 150 nM, about 200 nM to about 100 µM, about 200 nM to about 95 µM, about 200 nM to about 90 µM, about 200 nM to about 85 µM, about 200 nM to about 80 µM, about 200 nM to about 75 µM, about 200 nM to about 70 µM, about 200 nM to about 65 µM, about 200 nM to about 60 µM, about 200 nM to about 55 µM, about 200 nM to about 50 µM, about 200 nM to about 45 µM, about 200 nM to about 40 µM, about 200 nM to about 45 µM, about 200 nM to about 40 µM, about 200 nM to about 35 µM, about 200 nM to about 30 µM, about 200 nM to about 25 µM, about 200 nM to about 20 µM, about 200 nM to about 15 µM, about 200 nM to about 10 µM, about 200 nM to about 5 µM, about 200 nM to about 2 µM, about 200 nM to about 1 µM, about 200 nM to about 950 nM, about 200 nM to about 900 nM, about 200 nM to about 850 nM, about 200 nM to about 800 nM, about 200 nM to about 750 nM, about 200 nM to about 700 nM, about 200 nM to about 650 nM, about 200 nM to about 600 nM, about 200 nM to about 550 nM, about 200 nM to about 500 nM, about 200 nM to about 450 nM, about 200 nM to about 400 nM, about 200 nM to about 350 nM, about 200 nM to about 300 nM, about 200 nM to about 250 nM, about 300 nM to about 100 µM, about 300 nM to about 95 µM, about 300 nM to about 90 µM, about 300 nM to about 85 µM, about 300 nM to about 80 µM, about 300 nM to about 75 µM, about 300 nM to about 70 µM, about 300 nM to about 65 µM, about 300 nM to about 60 µM, about 300 nM to about 55 µM, about 300 nM to about 50 µM, about 300 nM to about 45 µM, about 300 nM to about 40 µM, about 300 nM to about 45 µM, about 300 nM to about 40 µM, about 300 nM to about 35 µM, about 300 nM to about 30 µM, about 300 nM to about 25 µM, about 300 nM to about 20 µM, about 300 nM to about 15 µM, about 300 nM to about 10 µM, about 300 nM to about 5 µM, about 300 nM to about 2 µM, about 300 nM to about 1 µM, about 300 nM to about 950 nM, about 300 nM to about 900 nM, about 300 nM to about 850 nM, about 300 nM to about 800 nM, about 300 nM to about 750 nM, about 300 nM to about 700 nM, about 300 nM to about 650 nM, about 300 nM to about 600 nM, about 300 nM to about 550 nM, about 300 nM to about 500 nM, about 300 nM to about 450 nM, about 300 nM to about 400 nM, about 300 nM to about 350 nM, about 400 nM to about 100 µM, about 400 nM to about 95 µM, about 400 nM to about 90 µM, about 400 nM to about 85 µM, about 400 nM to about 80 µM, about 400 nM to about 75 µM, about 400 nM to about 70 µM, about 400 nM to about 65 µM, about 400 nM to about 60 µM, about 400 nM to about 55 µM, about 400 nM to about 50 µM, about 400 nM to about 45 µM, about 400 nM to about 40 µM, about 400 nM to about 45 µM, about 400 nM to about 40 µM, about 400 nM to about 35 µM, about 400 nM to about 30 µM, about 400 nM to about 25 µM, about 400 nM to about 20 µM, about 400 nM to about 15 µM, about 400 nM to about 10 µM, about 400 nM to about 5 µM, about 400 nM to about 2 µM, about 400 nM to about 1 µM, about 400 nM to about 950 nM, about 400 nM to about 900 nM, about 400 nM to about 850 nM, about 400 nM to about 800 nM, about 400 nM to about 750 nM, about 400 nM to about 700 nM, about 400 nM to about 650 nM, about 400 nM to about 600 nM, about 400 nM to about 550 nM, about 400 nM to about 500 nM, about 400 nM to about 450 nM, about 500 nM to about 100 µM, about 500 nM to about 95 µM, about 500 nM to about 90 µM, about 500 nM to about 85 µM, about 500 nM to about 80 µM, about 500 nM to about 75 µM, about 500 nM to about 70 µM, about 500 nM to about 65 µM, about 500 nM to about 60 µM, about 500 nM to about 55 µM, about 500 nM to about 50 µM, about 500 nM to about 45 µM, about 500 nM to about 40 µM, about 500 nM to about 45 µM, about 500 nM to about 40 µM, about 500 nM to about 35 µM, about 500 nM to about 30 µM, about 500 nM to about 25 µM, about 500 nM to about 20 µM, about 500 nM to about 15 µM, about 500 nM to about 10 µM, about 500 nM to about 5 µM, about 500 nM to about 2 µM, about 500 nM to about 1 µM, about 500 nM to about 950 nM, about 500 nM to about 900 nM, about 500 nM to about 850 nM, about 500 nM to about 800 nM, about 500 nM to about 750 nM, about 500 nM to about 700 nM, about 500 nM to about 650 nM, about 500 nM to about 600 nM, about 500 nM to about 550 nM, about 600 nM to about 100 µM, about 600 nM to about 95 µM, about 600 nM to about 90 µM, about 600 nM to about 85 µM, about 600 nM to about 80 µM, about 600 nM to about 75 µM, about 600 nM to about 70 µM, about 600 nM to about 65 µM, about 600 nM to about 60 µM, about 600 nM to about 55 µM, about 600 nM to about 50 µM, about 600 nM to about 45 µM, about 600 nM to about 40 µM, about 600 nM to about 45 µM, about 600 nM to about 40 µM, about 600 nM to about 35 µM, about 600 nM to about 30 µM, about 600 nM to about 25 µM, about 600 nM to about 20 µM, about 600 nM to about 15 µM, about 600 nM to about 10 µM, about 600 nM to about 5 µM, about 600 nM to about 2 µM, about 600 nM to about 1 µM, about 600 nM to about 950 nM, about 600 nM to about 900 nM, about 600 nM to about 850 nM, about 600 nM to about 800 nM, about 600 nM to about 750 nM, about 600 nM to about 700 nM, about 600 nM to about 650 nM, about 700 nM to about 100 µM, about 700 nM to about 95 µM, about 700 nM to about 90 µM, about 700 nM to about 85 µM, about 700 nM to about 80 µM, about 700 nM to about 75 µM, about 700 nM to about 70 µM, about 700 nM to about 65 µM, about 700 nM to about 60 µM, about 700 nM to about 55 µM, about 700 nM to about 50 µM, about 700 nM to about 45 µM, about 700 nM to about 40 µM, about 700 nM to about 45 µM, about 700 nM to about 40 µM, about 700 nM to about 35 µM, about 700 nM to about 30 µM, about 700 nM to about 25 µM, about 700 nM to about 20 µM, about 700 nM to about 15 µM, about 700 nM to about 10 µM, about 700 nM to about 5 µM, about 700 nM to about 2 µM, about 700 nM to about 1 µM, about 700 nM to about 950 nM, about 700 nM to about 900 nM, about 700 nM to about 850 nM, about 700 nM to about 800 nM, about 700 nM to about 750 nM, about 800 nM to about 100 µM, about 800 nM to about 95 µM, about 800 nM to about 90 µM, about 800 nM to about 85 µM, about 800 nM to about 80 µM, about 800 nM to about 75 µM, about 800 nM to about 70 µM, about 800 nM to about 65 µM, about 800 nM to about 60 µM, about 800 nM to about 55 µM, about 800 nM to about 50 µM, about 800 nM to about 45 µM, about 800 nM to about 40 µM, about 800 nM to about 45 µM, about 800 nM to about 40 µM, about 800 nM to about 35 µM, about 800 nM to about 30 µM, about 800 nM to about 25 µM, about 800 nM to about 20 µM, about 800 nM to about 15 µM, about 800 nM to about 10 µM, about 800 nM to about 5 µM, about 800 nM to about 2 µM, about 800 nM to about 1 µM, about 800 nM to about 950 nM, about 800 nM to about 900 nM, about 800 nM to about 850 nM, about 900 nM to about 100 µM, about 900 nM to about 95 µM, about 900 nM to about 90 µM, about 900 nM to about 85 µM, about 900 nM to about 80 µM, about 900 nM to about 75 µM, about 900 nM to about 70 µM, about 900 nM to about 65 µM, about 900 nM to about 60 µM, about 900 nM to about 55 µM, about 900 nM to about 50 µM, about 900 nM to about 45 µM, about 900 nM to about 40 µM, about 900 nM to about 45 µM, about 900 nM to about 40 µM, about 900 nM to about 35 µM, about 900 nM to about 30 µM, about 900 nM to about 25 µM, about 900 nM to about 20 µM, about 900 nM to about 15 µM, about 900 nM to about 10 µM, about 900 nM to about 5 µM, about 900 nM to about 2 µM, about 900 nM to about 1 µM, about 900 nM to about 950 nM, about 1 µM to about 100 µM, about 1 µM to about 95 µM, about 1 µM to about 90 µM, about 1 µM to about 85 µM, about 1 µM to about 80 µM, about 1 µM to about 75 µM, about 1 µM to about 70 µM, about 1 µM to about 65 µM, about 1 µM to about 60 µM, about 1 µM to about 55 µM, about 1 µM to about 50 µM, about 1 µM to about 45 µM, about 1 µM to about 40 µM, about 1 µM to about 45 µM, about 1 µM to about 40 µM, about 1 µM to about 35 µM, about 1 µM to about 30 µM, about 1 µM to about 25 µM, about 1 µM to about 20 µM, about 1 µM to about 15 µM, about 1 µM to about 10 µM, about 1 µM to about 5 µM, about 1 µM to about 2 µM, about µM to about 100 µM, about µM to about 95 µM, about µM to about 90 µM, about µM to about 85 µM, about µM to about 80 µM, about µM to about 75 µM, about µM to about 70 µM, about µM to about 65 µM, about µM to about 60 µM, about µM to about 55 µM, about µM to about 50 µM, about µM to about 45 µM, about µM to about 40 µM, about µM to about 45 µM, about µM to about 40 µM, about µM to about 35 µM, about µM to about 30 µM, about µM to about 25 µM, about µM to about 20 µM, about µM to about 15 µM, about µM to about 10 µM, about µM to about 5 µM, about 5 µM to about 100 µM, about 5 µM to about 95 µM, about 5 µM to about 90 µM, about 5 µM to about 85 µM, about 5 µM to about 80 µM, about 5 µM to about 75 µM, about 5 µM to about 70 µM, about 5 µM to about 65 µM, about 5 µM to about 60 µM, about 5 µM to about 55 µM, about 5 µM to about 50 µM, about 5 µM to about 45 µM, about 5 µM to about 40 µM, about 5 µM to about 45 µM, about 5 µM to about 40 µM, about 5 µM to about 35 µM, about 5 µM to about 30 µM, about 5 µM to about 25 µM, about 5 µM to about 20 µM, about 5 µM to about 15 µM, about 5 µM to about 10 µM, about 10 µM to about 100 µM, about 10 µM to about 95 µM, about 10 µM to about 90 µM, about 10 µM to about 85 µM, about 10 µM to about 80 µM, about 10 µM to about 75 µM, about 10 µM to about 70 µM, about 10 µM to about 65 µM, about 10 µM to about 60 µM, about 10 µM to about 55 µM, about 10 µM to about 50 µM, about 10 µM to about 45 µM, about 10 µM to about 40 µM, about 10 µM to about 45 µM, about 10 µM to about 40 µM, about 10 µM to about 35 µM, about 10 µM to about 30 µM, about 10 µM to about 25 µM, about 10 µM to about 20 µM, about 10 µM to about 15 µM, about 20 µM to about 100 µM, about 20 µM to about 95 µM, about 20 µM to about 90 µM, about 20 µM to about 85 µM, about 20 µM to about 80 µM, about 20 µM to about 75 µM, about 20 µM to about 70 µM, about 20 µM to about 65 µM, about 20 µM to about 60 µM, about 20 µM to about 55 µM, about 20 µM to about 50 µM, about 20 µM to about 45 µM, about 20 µM to about 40 µM, about 20 µM to about 45 µM, about 20 µM to about 40 µM, about 20 µM to about 35 µM, about 20 µM to about 30 µM, about 20 µM to about 25 µM, about 30 µM to about 100 µM, about 30 µM to about 95 µM, about 30 µM to about 90 µM, about 30 µM to about 85 µM, about 30 µM to about 80 µM, about 30 µM to about 75 µM, about 30 µM to about 70 µM, about 30 µM to about 65 µM, about 30 µM to about 60 µM, about 30 µM to about 55 µM, about 30 µM to about 50 µM, about 30 µM to about 45 µM, about 30 µM to about 40 µM, about 30 µM to about 45 µM, about 30 µM to about 40 µM, about 30 µM to about 35 µM, about 40 µM to about 100 µM, about 40 µM to about 95 µM, about 40 µM to about 90 µM, about 40 µM to about 85 µM, about 40 µM to about 80 µM, about 40 µM to about 75 µM, about 40 µM to about 70 µM, about 40 µM to about 65 µM, about 40 µM to about 60 µM, about 40 µM to about 55 µM, about 40 µM to about 50 µM, about 40 µM to about 45 µM, about 50 µM to about 100 µM, about 50 µM to about 95 µM, about 50 µM to about 90 µM, about 50 µM to about 85 µM, about 50 µM to about 80 µM, about 50 µM to about 75 µM, about 50 µM to about 70 µM, about 50 µM to about 65 µM, about 50 µM to about 60 µM, about 50 µM to about 55 µM, about 60 µM to about 100 µM, about 60 µM to about 95 µM, about 60 µM to about 90 µM, about 60 µM to about 85 µM, about 60 µM to about 80 µM, about 60 µM to about 75 µM, about 60 µM to about 70 µM, about 60 µM to about 65 µM, about 70 µM to about 100 µM, about 70 µM to about 95 µM, about 70 µM to about 90 µM, about 70 µM to about 85 µM, about 70 µM to about 80 µM, about 70 µM to about 75 µM, about 80 µM to about 100 µM, about 80 µM to about 95 µM, about 80 µM to about 90 µM, about 80 µM to about 85 µM, about 90 µM to about 100 µM, or about 90 µM to about 95 µM) (e.g., as measured using SPR in phosphate buffered saline).

In some embodiments, the chimeric polypeptides described herein can have a total length of about 20 amino acids to about 1400 amino acids, about 20 amino acids to about 1350 amino acids, about 20 amino acids to about 1300 amino acids, about 20 amino acids to about 1250 amino acids, about 20 amino acids to about 1200 amino acids, about 20 amino acids to about 1150 amino acids, about 20 amino acids to about 1100 amino acids, about 20 amino acid to about 1050 amino acids, about 20 amino acids to about 1000 amino acids, about 20 amino acids to about 950 amino acids, about 20 amino acids to about 900 amino acids, about 20 amino acids to about 850 amino acids, about 20 amino acids to about 800 amino acids, about 20 amino acids to about 750 amino acids, about 20 amino acids to about 700 amino acids, about 20 amino acids to about 650 amino acids, about 20 amino acids to about 600 amino acids, about 20 amino acids to about 550 amino acids, about 20 amino acids to about 500 amino acids, about 20 amino acids to about 450 amino acids, about 20 amino acids to about 400 amino acids, about 20 amino acids to about 350 amino acids, about 20 amino acids to about 300 amino acids, about 20 amino acids to about 250 amino acids, about 20 amino acids to about 200 amino acids, about 20 amino acids to about 150 amino acids, about 20 amino acids to about 100 amino acids, about 20 amino acids to about 90 amino acids, about 20 amino acids to about 80 amino acids, about 20 amino acids to about 70 amino acids, about 20 amino acids to about 60 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 1400 amino acids, about 25 amino acids to about 1350 amino acids, about 25 amino acids to about 1300 amino acids, about 25 amino acids to about 1250 amino acids, about 25 amino acids to about 1200 amino acids, about 25 amino acids to about 1150 amino acids, about 25 amino acids to about 1100 amino acids, about 25 amino acid to about 1050 amino acids, about 25 amino acids to about 1000 amino acids, about 25 amino acids to about 950 amino acids, about 25 amino acids to about 900 amino acids, about 25 amino acids to about 850 amino acids, about 25 amino acids to about 800 amino acids, about 25 amino acids to about 750 amino acids, about 25 amino acids to about 700 amino acids, about 25 amino acids to about 650 amino acids, about 25 amino acids to about 600 amino acids, about 25 amino acids to about 550 amino acids, about 25 amino acids to about 500 amino acids, about 25 amino acids to about 450 amino acids, about 25 amino acids to about 400 amino acids, about 25 amino acids to about 350 amino acids, about 25 amino acids to about 300 amino acids, about 25 amino acids to about 250 amino acids, about 25 amino acids to about 200 amino acids, about 25 amino acids to about 150 amino acids, about 25 amino acids to about 100 amino acids, about 25 amino acids to about 90 amino acids, about 25 amino acids to about 80 amino acids, about 25 amino acids to about 70 amino acids, about 25 amino acids to about 60 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 1400 amino acids, about 30 amino acids to about 1350 amino acids, about 30 amino acids to about 1300 amino acids, about 30 amino acids to about 1250 amino acids, about 30 amino acids to about 1200 amino acids, about 30 amino acids to about 1150 amino acids, about 30 amino acids to about 1100 amino acids, about 30 amino acid to about 1050 amino acids, about 30 amino acids to about 1000 amino acids, about 30 amino acids to about 950 amino acids, about 30 amino acids to about 900 amino acids, about 30 amino acids to about 850 amino acids, about 30 amino acids to about 800 amino acids, about 30 amino acids to about 750 amino acids, about 30 amino acids to about 700 amino acids, about 30 amino acids to about 650 amino acids, about 30 amino acids to about 600 amino acids, about 30 amino acids to about 550 amino acids, about 30 amino acids to about 500 amino acids, about 30 amino acids to about 450 amino acids, about 30 amino acids to about 400 amino acids, about 30 amino acids to about 350 amino acids, about 30 amino acids to about 300 amino acids, about 30 amino acids to about 250 amino acids, about 30 amino acids to about 200 amino acids, about 30 amino acids to about 150 amino acids, about 30 amino acids to about 100 amino acids, about 30 amino acids to about 90 amino acids, about 30 amino acids to about 80 amino acids, about 30 amino acids to about 70 amino acids, about 30 amino acids to about 60 amino acids, about 30 amino acids to about 50 amino acids, about 30 amino acids to about 45 amino acids, about 30 amino acids to about 40 amino acids, about 30 amino acids to about 35 amino acids, about 35 amino acids to about 1400 amino acids, about 35 amino acids to about 1350 amino acids, about 35 amino acids to about 1300 amino acids, about 35 amino acids to about 1250 amino acids, about 35 amino acids to about 1200 amino acids, about 35 amino acids to about 1150 amino acids, about 35 amino acids to about 1100 amino acids, about 35 amino acid to about 1050 amino acids, about 35 amino acids to about 1000 amino acids, about 35 amino acids to about 950 amino acids, about 35 amino acids to about 900 amino acids, about 35 amino acids to about 850 amino acids, about 35 amino acids to about 800 amino acids, about 35 amino acids to about 750 amino acids, about 35 amino acids to about 700 amino acids, about 35 amino acids to about 650 amino acids, about 35 amino acids to about 600 amino acids, about 35 amino acids to about 550 amino acids, about 35 amino acids to about 500 amino acids, about 35 amino acids to about 450 amino acids, about 35 amino acids to about 400 amino acids, about 35 amino acids to about 350 amino acids, about 35 amino acids to about 300 amino acids, about 35 amino acids to about 250 amino acids, about 35 amino acids to about 200 amino acids, about 35 amino acids to about 150 amino acids, about 35 amino acids to about 100 amino acids, about 35 amino acids to about 90 amino acids, about 35 amino acids to about 80 amino acids, about 35 amino acids to about 70 amino acids, about 35 amino acids to about 60 amino acids, about 35 amino acids to about 50 amino acids, about 35 amino acids to about 45 amino acids, about 35 amino acids to about 40 amino acids, about 40 amino acids to about 1400 amino acids, about 40 amino acids to about 1350 amino acids, about 40 amino acids to about 1300 amino acids, about 40 amino acids to about 1250 amino acids, about 40 amino acids to about 1200 amino acids, about 40 amino acids to about 1150 amino acids, about 40 amino acids to about 1100 amino acids, about 40 amino acid to about 1050 amino acids, about 40 amino acids to about 1000 amino acids, about 40 amino acids to about 950 amino acids, about 40 amino acids to about 900 amino acids, about 40 amino acids to about 850 amino acids, about 40 amino acids to about 800 amino acids, about 40 amino acids to about 750 amino acids, about 40 amino acids to about 700 amino acids, about 40 amino acids to about 650 amino acids, about 40 amino acids to about 600 amino acids, about 40 amino acids to about 550 amino acids, about 40 amino acids to about 500 amino acids, about 40 amino acids to about 450 amino acids, about 40 amino acids to about 400 amino acids, about 40 amino acids to about 350 amino acids, about 40 amino acids to about 300 amino acids, about 40 amino acids to about 250 amino acids, about 40 amino acids to about 200 amino acids, about 40 amino acids to about 150 amino acids, about 40 amino acids to about 100 amino acids, about 40 amino acids to about 90 amino acids, about 40 amino acids to about 80 amino acids, about 40 amino acids to about 70 amino acids, about 40 amino acids to about 60 amino acids, about 40 amino acids to about 50 amino acids, about 40 amino acids to about 45 amino acids, about 45 amino acids to about 1400 amino acids, about 45 amino acids to about 1350 amino acids, about 45 amino acids to about 1300 amino acids, about 45 amino acids to about 1250 amino acids, about 45 amino acids to about 1200 amino acids, about 45 amino acids to about 1150 amino acids, about 45 amino acids to about 1100 amino acids, about 45 amino acid to about 1050 amino acids, about 45 amino acids to about 1000 amino acids, about 45 amino acids to about 950 amino acids, about 45 amino acids to about 900 amino acids, about 45 amino acids to about 850 amino acids, about 45 amino acids to about 800 amino acids, about 45 amino acids to about 750 amino acids, about 45 amino acids to about 700 amino acids, about 45 amino acids to about 650 amino acids, about 45 amino acids to about 600 amino acids, about 45 amino acids to about 550 amino acids, about 45 amino acids to about 500 amino acids, about 45 amino acids to about 450 amino acids, about 45 amino acids to about 400 amino acids, about 45 amino acids to about 350 amino acids, about 45 amino acids to about 300 amino acids, about 45 amino acids to about 250 amino acids, about 45 amino acids to about 200 amino acids, about 45 amino acids to about 150 amino acids, about 45 amino acids to about 100 amino acids, about 45 amino acids to about 90 amino acids, about 45 amino acids to about 80 amino acids, about 45 amino acids to about 70 amino acids, about 45 amino acids to about 60 amino acids, about 45 amino acids to about 50 amino acids, about 50 amino acids to about 1400 amino acids, about 50 amino acids to about 1350 amino acids, about 50 amino acids to about 1300 amino acids, about 50 amino acids to about 1250 amino acids, about 50 amino acids to about 1200 amino acids, about 50 amino acids to about 1150 amino acids, about 50 amino acids to about 1100 amino acids, about 50 amino acid to about 1050 amino acids, about 50 amino acids to about 1000 amino acids, about 50 amino acids to about 950 amino acids, about 50 amino acids to about 900 amino acids, about 50 amino acids to about 850 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 250 amino acids, about 50 amino acids to about 200 amino acids, about 50 amino acids to about 150 amino acids, about 50 amino acids to about 100 amino acids, about 50 amino acids to about 90 amino acids, about 50 amino acids to about 80 amino acids, about 50 amino acids to about 70 amino acids, about 50 amino acids to about 60 amino acids, about 60 amino acids to about 1400 amino acids, about 60 amino acids to about 1350 amino acids, about 60 amino acids to about 1300 amino acids, about 60 amino acids to about 1250 amino acids, about 60 amino acids to about 1200 amino acids, about 60 amino acids to about 1150 amino acids, about 60 amino acids to about 1100 amino acids, about 60 amino acid to about 1050 amino acids, about 60 amino acids to about 1000 amino acids, about 60 amino acids to about 950 amino acids, about 60 amino acids to about 900 amino acids, about 60 amino acids to about 850 amino acids, about 60 amino acids to about 800 amino acids, about 60 amino acids to about 750 amino acids, about 60 amino acids to about 700 amino acids, about 60 amino acids to about 650 amino acids, about 60 amino acids to about 600 amino acids, about 60 amino acids to about 550 amino acids, about 60 amino acids to about 500 amino acids, about 60 amino acids to about 450 amino acids, about 60 amino acids to about 400 amino acids, about 60 amino acids to about 350 amino acids, about 60 amino acids to about 300 amino acids, about 60 amino acids to about 250 amino acids, about 60 amino acids to about 200 amino acids, about 60 amino acids to about 150 amino acids, about 60 amino acids to about 100 amino acids, about 60 amino acids to about 90 amino acids, about 60 amino acids to about 80 amino acids, about 60 amino acids to about 70 amino acids, about 70 amino acids to about 1400 amino acids, about 70 amino acids to about 1350 amino acids, about 70 amino acids to about 1300 amino acids, about 70 amino acids to about 1250 amino acids, about 70 amino acids to about 1200 amino acids, about 70 amino acids to about 1150 amino acids, about 70 amino acids to about 1100 amino acids, about 70 amino acid to about 1050 amino acids, about 70 amino acids to about 1000 amino acids, about 70 amino acids to about 950 amino acids, about 70 amino acids to about 900 amino acids, about 70 amino acids to about 850 amino acids, about 70 amino acids to about 800 amino acids, about 70 amino acids to about 750 amino acids, about 70 amino acids to about 700 amino acids, about 70 amino acids to about 650 amino acids, about 70 amino acids to about 600 amino acids, about 70 amino acids to about 550 amino acids, about 70 amino acids to about 500 amino acids, about 70 amino acids to about 450 amino acids, about 70 amino acids to about 400 amino acids, about 70 amino acids to about 350 amino acids, about 70 amino acids to about 300 amino acids, about 70 amino acids to about 250 amino acids, about 70 amino acids to about 200 amino acids, about 70 amino acids to about 150 amino acids, about 70 amino acids to about 100 amino acids, about 70 amino acids to about 90 amino acids, about 70 amino acids to about 80 amino acids, about 80 amino acids to about 1400 amino acids, about 80 amino acids to about 1350 amino acids, about 80 amino acids to about 1300 amino acids, about 80 amino acids to about 1250 amino acids, about 80 amino acids to about 1200 amino acids, about 80 amino acids to about 1150 amino acids, about 80 amino acids to about 1100 amino acids, about 80 amino acid to about 1050 amino acids, about 80 amino acids to about 1000 amino acids, about 80 amino acids to about 950 amino acids, about 80 amino acids to about 900 amino acids, about 80 amino acids to about 850 amino acids, about 80 amino acids to about 800 amino acids, about 80 amino acids to about 750 amino acids, about 80 amino acids to about 700 amino acids, about 80 amino acids to about 650 amino acids, about 80 amino acids to about 600 amino acids, about 80 amino acids to about 550 amino acids, about 80 amino acids to about 500 amino acids, about 80 amino acids to about 450 amino acids, about 80 amino acids to about 400 amino acids, about 80 amino acids to about 350 amino acids, about 80 amino acids to about 300 amino acids, about 80 amino acids to about 250 amino acids, about 80 amino acids to about 200 amino acids, about 80 amino acids to about 150 amino acids, about 80 amino acids to about 100 amino acids, about 80 amino acids to about 90 amino acids, about 90 amino acids to about 1400 amino acids, about 90 amino acids to about 1350 amino acids, about 90 amino acids to about 1300 amino acids, about 90 amino acids to about 1250 amino acids, about 90 amino acids to about 1200 amino acids, about 90 amino acids to about 1150 amino acids, about 90 amino acids to about 1100 amino acids, about 90 amino acid to about 1050 amino acids, about 90 amino acids to about 1000 amino acids, about 90 amino acids to about 950 amino acids, about 90 amino acids to about 900 amino acids, about 90 amino acids to about 850 amino acids, about 90 amino acids to about 800 amino acids, about 90 amino acids to about 750 amino acids, about 90 amino acids to about 700 amino acids, about 90 amino acids to about 650 amino acids, about 90 amino acids to about 600 amino acids, about 90 amino acids to about 550 amino acids, about 90 amino acids to about 500 amino acids, about 90 amino acids to about 450 amino acids, about 90 amino acids to about 400 amino acids, about 90 amino acids to about 350 amino acids, about 90 amino acids to about 300 amino acids, about 90 amino acids to about 250 amino acids, about 90 amino acids to about 200 amino acids, about 90 amino acids to about 150 amino acids, about 90 amino acids to about 100 amino acids, about 100 amino acids to about 1400 amino acids, about 100 amino acids to about 1350 amino acids, about 100 amino acids to about 1300 amino acids, about 100 amino acids to about 1250 amino acids, about 100 amino acids to about 1200 amino acids, about 100 amino acids to about 1150 amino acids, about 100 amino acids to about 1100 amino acids, about 100 amino acid to about 1050 amino acids, about 100 amino acids to about 1000 amino acids, about 100 amino acids to about 950 amino acids, about 100 amino acids to about 900 amino acids, about 100 amino acids to about 850 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 250 amino acids, about 100 amino acids to about 200 amino acids, about 100 amino acids to about 150 amino acids, about 150 amino acids to about 1400 amino acids, about 150 amino acids to about 1350 amino acids, about 150 amino acids to about 1300 amino acids, about 150 amino acids to about 1250 amino acids, about 150 amino acids to about 1200 amino acids, about 150 amino acids to about 1150 amino acids, about 150 amino acids to about 1100 amino acids, about 150 amino acid to about 1050 amino acids, about 150 amino acids to about 1000 amino acids, about 150 amino acids to about 950 amino acids, about 150 amino acids to about 900 amino acids, about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 1400 amino acids, about 200 amino acids to about 1350 amino acids, about 200 amino acids to about 1300 amino acids, about 200 amino acids to about 1250 amino acids, about 200 amino acids to about 1200 amino acids, about 200 amino acids to about 1150 amino acids, about 200 amino acids to about 1100 amino acids, about 200 amino acid to about 1050 amino acids, about 200 amino acids to about 1000 amino acids, about 200 amino acids to about 950 amino acids, about 200 amino acids to about 900 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 1400 amino acids, about 250 amino acids to about 1350 amino acids, about 250 amino acids to about 1300 amino acids, about 250 amino acids to about 1250 amino acids, about 250 amino acids to about 1200 amino acids, about 250 amino acids to about 1150 amino acids, about 250 amino acids to about 1100 amino acids, about 250 amino acid to about 1050 amino acids, about 250 amino acids to about 1000 amino acids, about 250 amino acids to about 950 amino acids, about 250 amino acids to about 900 amino acids, about 250 amino acids to about 850 amino acids, about 250 amino acids to about 800 amino acids, about 250 amino acids to about 750 amino acids, about 250 amino acids to about 700 amino acids, about 250 amino acids to about 650 amino acids, about 250 amino acids to about 600 amino acids, about 250 amino acids to about 550 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 1400 amino acids, about 300 amino acids to about 1350 amino acids, about 300 amino acids to about 1300 amino acids, about 300 amino acids to about 1250 amino acids, about 300 amino acids to about 1200 amino acids, about 300 amino acids to about 1150 amino acids, about 300 amino acids to about 1100 amino acids, about 300 amino acid to about 1050 amino acids, about 300 amino acids to about 1000 amino acids, about 300 amino acids to about 950 amino acids, about 300 amino acids to about 900 amino acids, about 300 amino acids to about 850 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 1400 amino acids, about 350 amino acids to about 1350 amino acids, about 350 amino acids to about 1300 amino acids, about 350 amino acids to about 1250 amino acids, about 350 amino acids to about 1200 amino acids, about 350 amino acids to about 1150 amino acids, about 350 amino acids to about 1100 amino acids, about 350 amino acid to about 1050 amino acids, about 350 amino acids to about 1000 amino acids, about 350 amino acids to about 950 amino acids, about 350 amino acids to about 900 amino acids, about 350 amino acids to about 850 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 1400 amino acids, about 400 amino acids to about 1350 amino acids, about 400 amino acids to about 1300 amino acids, about 400 amino acids to about 1250 amino acids, about 400 amino acids to about 1200 amino acids, about 400 amino acids to about 1150 amino acids, about 400 amino acids to about 1100 amino acids, about 400 amino acid to about 1050 amino acids, about 400 amino acids to about 1000 amino acids, about 400 amino acids to about 950 amino acids, about 400 amino acids to about 900 amino acids, about 400 amino acids to about 850 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 1400 amino acids, about 450 amino acids to about 1350 amino acids, about 450 amino acids to about 1300 amino acids, about 450 amino acids to about 1250 amino acids, about 450 amino acids to about 1200 amino acids, about 450 amino acids to about 1150 amino acids, about 450 amino acids to about 1100 amino acids, about 450 amino acid to about 1050 amino acids, about 450 amino acids to about 1000 amino acids, about 450 amino acids to about 950 amino acids, about 450 amino acids to about 900 amino acids, about 450 amino acids to about 850 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 1400 amino acids, about 500 amino acids to about 1350 amino acids, about 500 amino acids to about 1300 amino acids, about 500 amino acids to about 1250 amino acids, about 500 amino acids to about 1200 amino acids, about 500 amino acids to about 1150 amino acids, about 500 amino acids to about 1100 amino acids, about 500 amino acid to about 1050 amino acids, about 500 amino acids to about 1000 amino acids, about 500 amino acids to about 950 amino acids, about 500 amino acids to about 900 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 1400 amino acids, about 550 amino acids to about 1350 amino acids, about 550 amino acids to about 1300 amino acids, about 550 amino acids to about 1250 amino acids, about 550 amino acids to about 1200 amino acids, about 550 amino acids to about 1150 amino acids, about 550 amino acids to about 1100 amino acids, about 550 amino acid to about 1050 amino acids, about 550 amino acids to about 1000 amino acids, about 550 amino acids to about 950 amino acids, about 550 amino acids to about 900 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 1400 amino acids, about 600 amino acids to about 1350 amino acids, about 600 amino acids to about 1300 amino acids, about 600 amino acids to about 1250 amino acids, about 600 amino acids to about 1200 amino acids, about 600 amino acids to about 1150 amino acids, about 600 amino acids to about 1100 amino acids, about 600 amino acid to about 1050 amino acids, about 600 amino acids to about 1000 amino acids, about 600 amino acids to about 950 amino acids, about 600 amino acids to about 900 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 1400 amino acids, about 650 amino acids to about 1350 amino acids, about 650 amino acids to about 1300 amino acids, about 650 amino acids to about 1250 amino acids, about 650 amino acids to about 1200 amino acids, about 650 amino acids to about 1150 amino acids, about 650 amino acids to about 1100 amino acids, about 650 amino acid to about 1050 amino acids, about 650 amino acids to about 1000 amino acids, about 650 amino acids to about 950 amino acids, about 650 amino acids to about 900 amino acids, about 650 amino acids to about 850 amino acids, about 650 amino acids to about 800 amino acids, about 650 amino acids to about 750 amino acids, about 650 amino acids to about 700 amino acids, about 700 amino acids to about 1400 amino acids, about 700 amino acids to about 1350 amino acids, about 700 amino acids to about 1300 amino acids, about 700 amino acids to about 1250 amino acids, about 700 amino acids to about 1200 amino acids, about 700 amino acids to about 1150 amino acids, about 700 amino acids to about 1100 amino acids, about 700 amino acid to about 1050 amino acids, about 700 amino acids to about 1000 amino acids, about 700 amino acids to about 950 amino acids, about 700 amino acids to about 900 amino acids, about 700 amino acids to about 850 amino acids, about 700 amino acids to about 800 amino acids, about 700 amino acids to about 750 amino acids, about 750 amino acids to about 1400 amino acids, about 750 amino acids to about 1350 amino acids, about 750 amino acids to about 1300 amino acids, about 750 amino acids to about 1250 amino acids, about 750 amino acids to about 1200 amino acids, about 750 amino acids to about 1150 amino acids, about 750 amino acids to about 1100 amino acids, about 750 amino acid to about 1050 amino acids, about 750 amino acids to about 1000 amino acids, about 750 amino acids to about 950 amino acids, about 750 amino acids to about 900 amino acids, about 750 amino acids to about 850 amino acids, about 750 amino acids to about 800 amino acids, about 800 amino acids to about 1400 amino acids, about 800 amino acids to about 1350 amino acids, about 800 amino acids to about 1300 amino acids, about 800 amino acids to about 1250 amino acids, about 800 amino acids to about 1200 amino acids, about 800 amino acids to about 1150 amino acids, about 800 amino acids to about 1100 amino acids, about 800 amino acid to about 1050 amino acids, about 800 amino acids to about 1000 amino acids, about 800 amino acids to about 950 amino acids, about 800 amino acids to about 900 amino acids, about 800 amino acids to about 850 amino acids, about 850 amino acids to about 1400 amino acids, about 850 amino acids to about 1350 amino acids, about 850 amino acids to about 1300 amino acids, about 850 amino acids to about 1250 amino acids, about 850 amino acids to about 1200 amino acids, about 850 amino acids to about 1150 amino acids, about 850 amino acids to about 1100 amino acids, about 850 amino acid to about 1050 amino acids, about 850 amino acids to about 1000 amino acids, about 850 amino acids to about 950 amino acids, about 850 amino acids to about 900 amino acids, about 900 amino acids to about 1400 amino acids, about 900 amino acids to about 1350 amino acids, about 900 amino acids to about 1300 amino acids, about 900 amino acids to about 1250 amino acids, about 900 amino acids to about 1200 amino acids, about 900 amino acids to about 1150 amino acids, about 900 amino acids to about 1100 amino acids, about 900 amino acid to about 1050 amino acids, about 900 amino acids to about 1000 amino acids, about 900 amino acids to about 950 amino acids, about 950 amino acids to about 1400 amino acids, about 950 amino acids to about 1350 amino acids, about 950 amino acids to about 1300 amino acids, about 950 amino acids to about 1250 amino acids, about 950 amino acids to about 1200 amino acids, about 950 amino acids to about 1150 amino acids, about 950 amino acids to about 1100 amino acids, about 950 amino acid to about 1050 amino acids, about 950 amino acids to about 1000 amino acids, about 1000 amino acids to about 1400 amino acids, about 1000 amino acids to about 1350 amino acids, about 1000 amino acids to about 1300 amino acids, about 1000 amino acids to about 1250 amino acids, about 1000 amino acids to about 1200 amino acids, about 1000 amino acids to about 1150 amino acids, about 1000 amino acids to about 1100 amino acids, about 1000 amino acid to about 1050 amino acids, about 1050 amino acids to about 1400 amino acids, about 1050 amino acids to about 1350 amino acids, about 1050 amino acids to about 1300 amino acids, about 1050 amino acids to about 1250 amino acids, about 1050 amino acids to about 1200 amino acids, about 1050 amino acids to about 1150 amino acids, about 1050 amino acids to about 1100 amino acids, about 1100 amino acids to about 1400 amino acids, about 1100 amino acids to about 1350 amino acids, about 1100 amino acids to about 1300 amino acids, about 1100 amino acids to about 1250 amino acids, about 1100 amino acids to about 1200 amino acids, about 1100 amino acids to about 1150 amino acids, about 1150 amino acids to about 1400 amino acids, about 1150 amino acids to about 1350 amino acids, about 1150 amino acids to about 1300 amino acids, about 1150 amino acids to about 1250 amino acids, about 1150 amino acids to about 1200 amino acids, about 1200 amino acids to about 1400 amino acids, about 1200 amino acids to about 1350 amino acids, about 1200 amino acids to about 1300 amino acids, about 1200 amino acids to about 1250 amino acids, about 1250 amino acids to about 1400 amino acids, about 1250 amino acids to about 1350 amino acids, about 1250 amino acids to about 1300 amino acids, about 1300 amino acids to about 1400 amino acids, about 1300 amino acids to about 1350 amino acids, or about 1350 amino acids to about 1400 amino acids.

For example, the chimeric polypeptide may include: LLADTTHHRPWTVIGESTHHRPWSIIGESSHHKPFTGLGDTTHHRPWGILAESTHHKPWTA▫TGGSGEGGTGASTGGSAGTGGSGGTTSGEAGGSSGAG (SEQ ID NO: 44). In some instances the chimeric polypeptide includes a β-TCP binding sequence and a linker that has an amino acid sequence that has at least 70% (e.g., at least 75%, at least 80%, at least 85%, at least 90%, at least 95%, at least 96%, at least 97%, at least 98%, at least 99%) sequence identity to SEQ ID NO: 44.

For example, the chimeric polypeptide may include: LLADTTHHRPWTGLGDTTHHRPWGLLADTTHHRPWTGLGDTTHHRPWGLLADTTHHRPWTTGGSGEGGTGASTGGSAGTGGSGGTTSGEAGGSSGAG (SEQ ID NO: 45.In some instances the chimeric polypeptide includes a β-TCP binding sequence and a linker that has an amino acid sequence that has at least 70% (e.g., at least 75%, at least 80%, at least 85%, at least 90%, at least 95%, at least 96%, at least 97%, at least 98%, at least 99%) sequence identity to SEQ ID NO: 45.

A variety of different methods known in the art can be used to determine the K_{D} values of any of the chimeric polypeptides described herein for binding to β-TCP (e.g., any of the types of β-TCP described herein) (e.g., an electrophoretic mobility shift assay, a filter binding assay, surface plasmon resonance, and a biomolecular binding kinetics assay, etc.)

The chimeric polypeptides provided herein are useful orthopedic materials, and can be used as bone void fillers and for bone reconstructions. The chimeric polypeptides described herein are osteo-conductive and easily resorbed.

### Linkers

In some instances, a neighboring pair (or each neighboring pair) of two β-TCP binding sequences directly abut each other. In other instances, a neighboring pair (or each neighboring pair) of two β-TCP binding sequences are separated by a linker sequence. For example, a linker sequence can be 1 amino acid to about 100 amino acids, 1 amino acid to about 95 amino acids, 1 amino acid to about 90 amino acids, 1 amino acid to about 85 amino acids, 1 amino acid to about 80 amino acids, 1 amino acid to about 75 amino acids, 1 amino acid to about 70 amino acids, 1 amino acid to about 65 amino acids, 1 amino acid to about 60 amino acids, 1 amino acid to about 55 amino acids, 1 amino acid to about 50 amino acids, 1 amino acid to about 45 amino acids, 1 amino acid to about 40 amino acids, 1 amino acid to about 38 amino acids, 1 amino acid to about 36 amino acids, 1 amino acid to about 34 amino acids, 1 amino acid to about 32 amino acids, 1 amino acid to about 30 amino acids, 1 amino acid to about 28 amino acids, 1 amino acid to about 26 amino acids, 1 amino acid to about 24 amino acids, 1 amino acid to about 22 amino acids, 1 amino acid to about 20 amino acids, 1 amino acid to about 18 amino acids, 1 amino acid to about 16 amino acids, 1 amino acid to about 14 amino acids, 1 amino acid to about 12 amino acids, 1 amino acid to about 10 amino acids, 1 amino acid to about 8 amino acids, 1 amino acid to about 6 amino acids, 1 amino acid to about 4 amino acids, about 2 amino acids to about 100 amino acids, about 2 amino acids to about 95 amino acids, about 2 amino acids to about 90 amino acids, about 2 amino acids to about 85 amino acids, about 2 amino acids to about 80 amino acids, about 2 amino acids to about 75 amino acids, about 2 amino acids to about 70 amino acids, about 2 amino acids to about 65 amino acids, about 2 amino acids to about 60 amino acids, about 2 amino acids to about 55 amino acids, about 2 amino acids to about 50 amino acids, about 2 amino acids to about 45 amino acids, about 2 amino acids to about 40 amino acids, about 2 amino acids to about 38 amino acids, about 2 amino acids to about 36 amino acids, about 2 amino acids to about 34 amino acids, about 2 amino acids to about 32 amino acids, about 2 amino acids to about 30 amino acids, about 2 amino acids to about 28 amino acids, about 2 amino acids to about 26 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 4 amino acids, about 4 amino acids to about 100 amino acids, about 4 amino acids to about 95 amino acids, about 4 amino acids to about 90 amino acids, about 4 amino acids to about 85 amino acids, about 4 amino acids to about 80 amino acids, about 4 amino acids to about 75 amino acids, about 4 amino acids to about 70 amino acids, about 4 amino acids to about 65 amino acids, about 4 amino acids to about 60 amino acids, about 4 amino acids to about 55 amino acids, about 4 amino acids to about 50 amino acids, about 4 amino acids to about 45 amino acids, about 4 amino acids to about 40 amino acids, about 4 amino acids to about 38 amino acids, about 4 amino acids to about 36 amino acids, about 4 amino acids to about 34 amino acids, about 4 amino acids to about 32 amino acids, about 4 amino acids to about 30 amino acids, about 4 amino acids to about 28 amino acids, about 4 amino acids to about 26 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 6 amino acids to about 100 amino acids, about 6 amino acids to about 95 amino acids, about 6 amino acids to about 90 amino acids, about 6 amino acids to about 85 amino acids, about 6 amino acids to about 80 amino acids, about 6 amino acids to about 75 amino acids, about 6 amino acids to about 70 amino acids, about 6 amino acids to about 65 amino acids, about 6 amino acids to about 60 amino acids, about 6 amino acids to about 55 amino acids, about 6 amino acids to about 50 amino acids, about 6 amino acids to about 45 amino acids, about 6 amino acids to about 40 amino acids, about 6 amino acids to about 38 amino acids, about 6 amino acids to about 36 amino acids, about 6 amino acids to about 34 amino acids, about 6 amino acids to about 32 amino acids, about 6 amino acids to about 30 amino acids, about 6 amino acids to about 28 amino acids, about 6 amino acids to about 26 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 14 amino acids, about 6 amino acids to about 12 amino acids, about 6 amino acids to about 10 amino acids, about 6 amino acids to about 8 amino acids, about 8 amino acids to about 100 amino acids, about 8 amino acids to about 95 amino acids, about 8 amino acids to about 90 amino acids, about 8 amino acids to about 85 amino acids, about 8 amino acids to about 80 amino acids, about 8 amino acids to about 75 amino acids, about 8 amino acids to about 70 amino acids, about 8 amino acids to about 65 amino acids, about 8 amino acids to about 60 amino acids, about 8 amino acids to about 55 amino acids, about 8 amino acids to about 50 amino acids, about 8 amino acids to about 45 amino acids, about 8 amino acids to about 40 amino acids, about 8 amino acids to about 38 amino acids, about 8 amino acids to about 36 amino acids, about 8 amino acids to about 34 amino acids, about 8 amino acids to about 32 amino acids, about 8 amino acids to about 30 amino acids, about 8 amino acids to about 28 amino acids, about 8 amino acids to about 26 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 14 amino acids, about 8 amino acids to about 12 amino acids, about 8 amino acids to about 10 amino acids, about 10 amino acids to about 100 amino acids, about 10 amino acids to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 38 amino acids, about 10 amino acids to about 36 amino acids, about 10 amino acids to about 34 amino acids, about 10 amino acids to about 32 amino acids, about 10 amino acids to about 30 amino acids, about 10 amino acids to about 28 amino acids, about 10 amino acids to about 26 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 14 amino acids, about 10 amino acids to about 12 amino acids, about 12 amino acids to about 100 amino acids, about 12 amino acids to about 95 amino acids, about 12 amino acids to about 90 amino acids, about 12 amino acids to about 85 amino acids, about 12 amino acids to about 80 amino acids, about 12 amino acids to about 75 amino acids, about 12 amino acids to about 70 amino acids, about 12 amino acids to about 65 amino acids, about 12 amino acids to about 60 amino acids, about 12 amino acids to about 55 amino acids, about 12 amino acids to about 50 amino acids, about 12 amino acids to about 45 amino acids, about 12 amino acids to about 40 amino acids, about 12 amino acids to about 38 amino acids, about 12 amino acids to about 36 amino acids, about 12 amino acids to about 34 amino acids, about 12 amino acids to about 32 amino acids, about 12 amino acids to about 30 amino acids, about 12 amino acids to about 28 amino acids, about 12 amino acids to about 26 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 14 amino acids, about 14 amino acids to about 100 amino acids, about 14 amino acids to about 95 amino acids, about 14 amino acids to about 90 amino acids, about 14 amino acids to about 85 amino acids, about 14 amino acids to about 80 amino acids, about 14 amino acids to about 75 amino acids, about 14 amino acids to about 70 amino acids, about 14 amino acids to about 65 amino acids, about 14 amino acids to about 60 amino acids, about 14 amino acids to about 55 amino acids, about 14 amino acids to about 50 amino acids, about 14 amino acids to about 45 amino acids, about 14 amino acids to about 40 amino acids, about 14 amino acids to about 38 amino acids, about 14 amino acids to about 36 amino acids, about 14 amino acids to about 34 amino acids, about 14 amino acids to about 32 amino acids, about 14 amino acids to about 30 amino acids, about 14 amino acids to about 28 amino acids, about 14 amino acids to about 26 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 16 amino acids to about 100 amino acids, about 16 amino acids to about 95 amino acids, about 16 amino acids to about 90 amino acids, about 16 amino acids to about 85 amino acids, about 16 amino acids to about 80 amino acids, about 16 amino acids to about 75 amino acids, about 16 amino acids to about 70 amino acids, about 16 amino acids to about 65 amino acids, about 16 amino acids to about 60 amino acids, about 16 amino acids to about 55 amino acids, about 16 amino acids to about 50 amino acids, about 16 amino acids to about 45 amino acids, about 16 amino acids to about 40 amino acids, about 16 amino acids to about 38 amino acids, about 16 amino acids to about 36 amino acids, about 16 amino acids to about 34 amino acids, about 16 amino acids to about 32 amino acids, about 16 amino acids to about 30 amino acids, about 16 amino acids to about 28 amino acids, about 16 amino acids to about 26 amino acids, about 16 amino acids to about 24 amino acids, about 16 amino acids to about 22 amino acids, about 16 amino acids to about 20 amino acids, about 16 amino acids to about 18 amino acids, about 18 amino acids to about 100 amino acids, about 18 amino acids to about 95 amino acids, about 18 amino acids to about 90 amino acids, about 18 amino acids to about 85 amino acids, about 18 amino acids to about 80 amino acids, about 18 amino acids to about 75 amino acids, about 18 amino acids to about 70 amino acids, about 18 amino acids to about 65 amino acids, about 18 amino acids to about 60 amino acids, about 18 amino acids to about 55 amino acids, about 18 amino acids to about 50 amino acids, about 18 amino acids to about 45 amino acids, about 18 amino acids to about 40 amino acids, about 18 amino acids to about 38 amino acids, about 18 amino acids to about 36 amino acids, about 18 amino acids to about 34 amino acids, about 18 amino acids to about 32 amino acids, about 18 amino acids to about 30 amino acids, about 18 amino acids to about 28 amino acids, about 18 amino acids to about 26 amino acids, about 18 amino acids to about 24 amino acids, about 18 amino acids to about 22 amino acids, about 18 amino acids to about 20 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 38 amino acids, about 20 amino acids to about 36 amino acids, about 20 amino acids to about 34 amino acids, about 20 amino acids to about 32 amino acids, about 20 amino acids to about 30 amino acids, about 20 amino acids to about 28 amino acids, about 20 amino acids to about 26 amino acids, about 20 amino acids to about 24 amino acids, about 20 amino acids to about 22 amino acids, about 22 amino acids to about 100 amino acids, about 22 amino acids to about 95 amino acids, about 22 amino acids to about 90 amino acids, about 22 amino acids to about 85 amino acids, about 22 amino acids to about 80 amino acids, about 22 amino acids to about 75 amino acids, about 22 amino acids to about 70 amino acids, about 22 amino acids to about 65 amino acids, about 22 amino acids to about 60 amino acids, about 22 amino acids to about 55 amino acids, about 22 amino acids to about 50 amino acids, about 22 amino acids to about 45 amino acids, about 22 amino acids to about 40 amino acids, about 22 amino acids to about 38 amino acids, about 22 amino acids to about 36 amino acids, about 22 amino acids to about 34 amino acids, about 22 amino acids to about 32 amino acids, about 22 amino acids to about 30 amino acids, about 22 amino acids to about 28 amino acids, about 22 amino acids to about 26 amino acids, about 22 amino acids to about 24 amino acids, about 24 amino acids to about 100 amino acids, about 24 amino acids to about 95 amino acids, about 24 amino acids to about 90 amino acids, about 24 amino acids to about 85 amino acids, about 24 amino acids to about 80 amino acids, about 24 amino acids to about 75 amino acids, about 24 amino acids to about 70 amino acids, about 24 amino acids to about 65 amino acids, about 24 amino acids to about 60 amino acids, about 24 amino acids to about 55 amino acids, about 24 amino acids to about 50 amino acids, about 24 amino acids to about 45 amino acids, about 24 amino acids to about 40 amino acids, about 24 amino acids to about 38 amino acids, about 24 amino acids to about 36 amino acids, about 24 amino acids to about 34 amino acids, about 24 amino acids to about 32 amino acids, about 24 amino acids to about 30 amino acids, about 24 amino acids to about 28 amino acids, about 24 amino acids to about 26 amino acids, about 26 amino acids to about 100 amino acids, about 26 amino acids to about 95 amino acids, about 26 amino acids to about 90 amino acids, about 26 amino acids to about 85 amino acids, about 26 amino acids to about 80 amino acids, about 26 amino acids to about 75 amino acids, about 26 amino acids to about 70 amino acids, about 26 amino acids to about 65 amino acids, about 26 amino acids to about 60 amino acids, about 26 amino acids to about 55 amino acids, about 26 amino acids to about 50 amino acids, about 26 amino acids to about 45 amino acids, about 26 amino acids to about 40 amino acids, about 26 amino acids to about 38 amino acids, about 26 amino acids to about 36 amino acids, about 26 amino acids to about 34 amino acids, about 26 amino acids to about 32 amino acids, about 26 amino acids to about 30 amino acids, about 26 amino acids to about 28 amino acids, about 28 amino acids to about 100 amino acids, about 28 amino acids to about 95 amino acids, about 28 amino acids to about 90 amino acids, about 28 amino acids to about 85 amino acids, about 28 amino acids to about 80 amino acids, about 28 amino acids to about 75 amino acids, about 28 amino acids to about 70 amino acids, about 28 amino acids to about 65 amino acids, about 28 amino acids to about 60 amino acids, about 28 amino acids to about 55 amino acids, about 28 amino acids to about 50 amino acids, about 28 amino acids to about 45 amino acids, about 28 amino acids to about 40 amino acids, about 28 amino acids to about 38 amino acids, about 28 amino acids to about 36 amino acids, about 28 amino acids to about 34 amino acids, about 28 amino acids to about 32 amino acids, about 28 amino acids to about 30 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 38 amino acids, about 30 amino acids to about 36 amino acids, about 30 amino acids to about 34 amino acids, about 30 amino acids to about 32 amino acids, about 32 amino acids to about 100 amino acids, about 32 amino acids to about 95 amino acids, about 32 amino acids to about 90 amino acids, about 32 amino acids to about 85 amino acids, about 32 amino acids to about 80 amino acids, about 32 amino acids to about 75 amino acids, about 32 amino acids to about 70 amino acids, about 32 amino acids to about 65 amino acids, about 32 amino acids to about 60 amino acids, about 32 amino acids to about 55 amino acids, about 32 amino acids to about 50 amino acids, about 32 amino acids to about 45 amino acids, about 32 amino acids to about 40 amino acids, about 32 amino acids to about 38 amino acids, about 32 amino acids to about 36 amino acids, about 32 amino acids to about 34 amino acids, about 34 amino acids to about 100 amino acids, about 34 amino acids to about 95 amino acids, about 34 amino acids to about 90 amino acids, about 34 amino acids to about 85 amino acids, about 34 amino acids to about 80 amino acids, about 34 amino acids to about 75 amino acids, about 34 amino acids to about 70 amino acids, about 34 amino acids to about 65 amino acids, about 34 amino acids to about 60 amino acids, about 34 amino acids to about 55 amino acids, about 34 amino acids to about 50 amino acids, about 34 amino acids to about 45 amino acids, about 34 amino acids to about 40 amino acids, about 34 amino acids to about 38 amino acids, about 34 amino acids to about 36 amino acids, about 36 amino acids to about 100 amino acids, about 36 amino acids to about 95 amino acids, about 36 amino acids to about 90 amino acids, about 36 amino acids to about 85 amino acids, about 36 amino acids to about 80 amino acids, about 36 amino acids to about 75 amino acids, about 36 amino acids to about 70 amino acids, about 36 amino acids to about 65 amino acids, about 36 amino acids to about 60 amino acids, about 36 amino acids to about 55 amino acids, about 36 amino acids to about 50 amino acids, about 36 amino acids to about 45 amino acids, about 36 amino acids to about 40 amino acids, about 36 amino acids to about 38 amino acids, about 38 amino acids to about 100 amino acids, about 38 amino acids to about 95 amino acids, about 38 amino acids to about 90 amino acids, about 38 amino acids to about 85 amino acids, about 38 amino acids to about 80 amino acids, about 38 amino acids to about 75 amino acids, about 38 amino acids to about 70 amino acids, about 38 amino acids to about 65 amino acids, about 38 amino acids to about 60 amino acids, about 38 amino acids to about 55 amino acids, about 38 amino acids to about 50 amino acids, about 38 amino acids to about 45 amino acids, about 38 amino acids to about 40 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 45 amino acids to about 100 amino acids, about 45 amino acids to about 95 amino acids, about 45 amino acids to about 90 amino acids, about 45 amino acids to about 85 amino acids, about 45 amino acids to about 80 amino acids, about 45 amino acids to about 75 amino acids, about 45 amino acids to about 70 amino acids, about 45 amino acids to about 65 amino acids, about 45 amino acids to about 60 amino acids, about 45 amino acids to about 55 amino acids, about 45 amino acids to about 50 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 55 amino acids to about 100 amino acids, about 55 amino acids to about 95 amino acids, about 55 amino acids to about 90 amino acids, about 55 amino acids to about 85 amino acids, about 55 amino acids to about 80 amino acids, about 55 amino acids to about 75 amino acids, about 55 amino acids to about 70 amino acids, about 55 amino acids to about 65 amino acids, about 55 amino acids to about 60 amino acids, about 60 amino acids to about 100 amino acids, about 60 amino acids to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 65 amino acids to about 100 amino acids, about 65 amino acids to about 95 amino acids, about 65 amino acids to about 90 amino acids, about 65 amino acids to about 85 amino acids, about 65 amino acids to about 80 amino acids, about 65 amino acids to about 75 amino acids, about 65 amino acids to about 70 amino acids, about 70 amino acids to about 100 amino acids, about 70 amino acids to about 95 amino acids, about 70 amino acids to about 90 amino acids, about 70 amino acids to about 85 amino acids, about 70 amino acids to about 80 amino acids, about 70 amino acids to about 75 amino acids, about 75 amino acids to about 100 amino acids, about 75 amino acids to about 95 amino acids, about 75 amino acids to about 90 amino acids, about 75 amino acids to about 85 amino acids, about 75 amino acids to about 80 amino acids, about 80 amino acids to about 100 amino acids, about 80 amino acids to about 95 amino acids, about 80 amino acids to about 90 amino acids, about 80 amino acids to about 85 amino acids, about 85 amino acids to about 100 amino acids, about 85 amino acids to about 95 amino acids, about 85 amino acids to about 90 amino acids, about 90 amino acids to about 100 amino acids, about 90 amino acids to about 95 amino acids, or about 95 amino acids to about 100 amino acids.

For example, a linker sequence can comprise or consist of TGGSGEGGTGASTGGSAGTGGSGGTTSGEAGGSSGAG (SEQ ID NO: 33) or GSGATG (SEQ ID NO: 34). In some instances the chimeric polypeptide includes a linker that has an amino acid sequence that has at least 70% (e.g., at least 75%, at least 80%, at least 85%, at least 90%, at least 95%, at least 96%, at least 97%, at least 98%, at least 99%) sequence identity to SEQ ID NO: 33. For example, a linker sequence can comprise or consist of the sequences of GSGS, GSGSGS, SGSG, SGSGSG, GSSG, SS, or GGGGS.

### Growth Factors

A β-TCP-binding sequence as described herein can be tethered to a mammalian growth factor. In some instances, a linker sequence (e.g., any of the linker sequences described herein or known in the art) is disposed between the β-TCP-binding sequence and the mammalian growth factor.

Mammalian growth factors can be osteoinductive molecules that are capable of initiating and enhancing the bone repair process. Bone morphogenetic proteins (BMP) represent a distinct subset of the transforming growth factor-β family. A number of these BMP (BMP-2, BMP-7, and BMP-14) enhance the speed of bone healing in defects and non-unions.

Non-limiting examples of mammalian growth factors are described herein. In some instances, the mammalian growth factor is selected from the group of: epidermal growth factor (EGF), platelet derived growth factor (PDGF), insulin like growth factor (IGF-1), fibroblast growth factor (FGF), fibroblast growth factor 2 (FGF2), fibroblast growth factor 18 (FGF18), transforming growth factor alpha (TGF-α), transforming growth factor beta (TGF-β), transforming growth factor beta 1 (TGF-β1), transforming growth factor beta 3 (TGF-β3), osteogenic protein 1 (OP-1), osteogenic protein 2 (OP-2), osteogenic protein 3 (OP-3), bone morphogenetic protein 2 (BMP-2), bone morphogenetic protein 3 (BMP-3), bone morphogenetic protein 4 (BMP-4), bone morphogenetic protein 5 (BMP-5), bone morphogenetic protein 6 (BMP-6), bone morphogenetic protein 7 (BMP-7), bone morphogenetic protein (BMP-9), bone morphogenetic protein 10 (BMP-10), bone morphogenetic protein 11 (BMP-11), bone morphogenetic protein 12 (BMP-12), bone morphogenetic protein 13 (BMP-13), bone morphogenetic protein 15 (BMP-15), dentin phosphoprotein (DPP), vegetal related growth factor (Vgr), growth differentiation factor 1 (GDF-1), growth differentiation factor 3 (GDF-3), growth differentiation factor 5 (GDF-5), growth differentiation factor 6 (GDF-6), growth differentiation factor 7 (GDF-7), growth differentiation factor 8 (GDF8), growth differentiation factor 11 (GDF11), growth differentiation factor 15 (GDF15), vascular endothelial growth factor (VEGF), hyaluronic acid binding protein (HABP), and collagen binding protein (CBP), fibroblast growth factor 18 (FGF-18), keratinocyte growth factor (KGF), tumor necrosis factor alpha (TNFα), tumor necrosis factor (TNF)- related apoptosis inducing ligand (TRAIL), wnt family member 1 (WNT1), wnt family member 2 (WNT2), wnt family member 2B (WNT2B), wnt family member 3 (WNT3), wnt family member 3A (WNT3A), wnt family member 4 (WNT4), wnt family member 5A (WNT5A), wnt family member 5B (WNT5B), wnt family member 6 (WNT6), wnt family member 7A (WNT7A), wnt family member 7B (WNT7B), wnt family member 8A (WNT8A), wnt family member 8B (WNT8B), wnt family member 9A (WNT9A), wnt family member 9B (WNT9B), wnt family member 10A (WNT10A), wnt family member 10B (WNT10B), wnt family member 11 (WNT11), and wnt family member 16 (WNT16).

For example, the mammalian growth factor can be a human growth factor. Non-limiting examples of human growth factors are provided in Table B.

TABLE B

Exemplary human growth factors

| Name | Exemplary Human Protein Sequence | SEQ ID NO: |
|---|---|---|
| EGF | MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLD | 46 |
| | KRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSW ECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRAL DPPHQMELTQ | |
| PDGF | MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTGRPRESGKKRKRKRLKPT | 47 |
| IGF-1 | MGKISSLPTQLFKCCFCDFLKVKMHTMSSSHLFYLALCLLTFTSSATAGPETLCGAELVDALQFVCGDRGFYFNKPTGYGSSSR | 48 |
| | RAPQTGIVDECCFRSCDLRRLEMYCAPLKPAKSARSVRAQRHTDMPKTQKYQPPSTNKNTKSQRRKGSTFEERK | |
| FGF | MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD | 49 |
| FGF2 | MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNT YRSRKYTSWYVALKRTGQYKLGSKTGPGQKAILFLPMSAKS | 50 |
| FGF18 | MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA | 51 |
| TGF-α | MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV | 52 |
| TGF-β1 | MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNC | 53 |
| | CVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS | |
| TGF-β3 | MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS | 54 |
| OP-2 (BMP-8) | MTALPGPLWLLGLALCALGGGGPGLRPPPGCPQRRLGARERRDVQREILAVLGLPGRPRPRAPPAASRLPASAPLFMLDLYHAMAGDDDEDGAPAERRLGRADLVMSFVNMVERDRALGHQEPHWKEFRFDLTQIPAGEAVTAAEFRIYKVPSIHLLNRTLHVSMFQVVQEQSNRESDLFFLDLQTLRAGDEGWLVLDVTAASDCWLLKRHKDLGLRLYVETEDGHSVDPGLAGLLGQRAPRSQQPFVVTFFRASPSPIRTPRAVRPLRRRQPKKSNELPQANRLPGIFDDVHGSHGRQVCRRHELYVSFQDLGWLDWVIAPQGYSAYYCEGECSFPLDSCMNATNHAILQSLVHLMMPDAVPKACCAPTKLSATSVLYYDSSNNVILRKHRNMVVKACGCH | 55 |
| OP-3 | MAARPGLLWLLGLALCVLGGGHLSHPPHVFPQRRLGVREPRDMQREIREVLGLPGRPRSRAPVGAAQQPASAPLFMLDLYRAMTDDSGGGTPQPHLDRADLIMSFVNIVERDRTLGYQEPHWKEFHFDLTQIPAGEAVTAAEFRIYKEPSTHPLNTTLHISMFEVVQEHSNRESDLFFLDLQTLRSGDEGWLVLDITAASDRWLLNHHKDLGLRLYVETEDGHSIDPGLAGLLGRQAPRSRQPFMVGFFRANQSPVRAPRTARPLKKKQLNQINQLPHSNKHLGILDDGHGSHGREVCRRHELYVSFRDLGWLDSVIAPQGYSAYYCAGEC | 56 |
| | IYPLNSCMNSTNHATMQALVHLMKPDIIPKVCCVPTELSAISLLYYDRNNNVILRRERNMVVQACGCH | |
| BMP-2 | MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR | 32 |
| BMP-3 | MAGASRLLFLWLGCFCVSLAQGERPKPPFPELRKAVPGDRTAGGGPDSELQPQDKVSEHMLRLYDRYSTVQAARTPGSLEGGSQPWRPRLLREGNTVRSFRAAAAETLERKGLYIFNLTSLTKSENILSATLYFCIGELGNISLSCPVSGGCSHHAQRKHIQIDLSAWTLKFSRNQSQLLGHLSVDMAKSHRDIMSWLSKDITQFLRKAKENEEFLIGFNITSKGRQLPKRRLPFPEPYILVYANDAAISEPESVVSSLQGHRNFPTGTVPKWDSHIRAALSIERRKKRSTGVLLPLQNNELPGAEYQYKKDEVWEERKPYKTLQAQAPEKSKNKKKQRKGPHRKSQTLQFDEQTLKKARRKQWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR | 57 |
| BMP-4 | MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKR | 58 |
| | SPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR | |
| BMP-5 | MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH | 59 |
| BMP-6/VGR | MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEF SPRQRHHKEFKFNLSQIPEG EVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH | 60 |
| BMP-7/OP-1 | MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGE | 61 |
| | AVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILK KYRNMVVRACGCH | |
| BMP-9 | MCPGALWVALPLLSLLAGSLQGKPLQSWGRGSAGGNAHSPLGVPGGGLPEHTFNLKMFLENVKVDFLRSLNLSGVPSQDKTRVEPPQYMIDLYNRYTSDKSTTPASNIVRSFSMEDAISITATEDFPFQKHILLFNISIPRHEQITRAELRLYVSCQNHVDPSHDLKGSVVIYDVLDGTDAWDSATETKTFLVSQDIQDEGWETLEVSSAVKRWVRSDSTKSKNKLEVTVESHRKGCDTLDISVPPGSRNLPFFVVFSNDHSSGTKETRLELREMISHEQESVLKKLSKDGSTEAGESSHEEDTDGHVAAGSTLARRKRSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR | 62 |
| BMP-10 | MGSLVLTLCALFCLAAYLVSGSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLI VFSDDQSSDKERKEELNEMISHEQLPELDNLGLDSFSSGPGEEALLQMRSNIIYDSTARIRRNAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR | 63 |
| BMP-11/GDF-11 | MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS | 64 |
| BMP-12 | MDLSAAAALCLWLLSACRPRDGLEAAAVLRAAGAGPVRSPGGGGGGGGGGRTLAQAAGAAAVPAAAVPRARAARRAAGSGFRNGSVVPHHFMMSLYRSLAGRAPAGAAAVSASGHGRADTITGFTDQATQDESAAETGQSFLFDVSSLNDADEVVGAELRVLRRGSPESGPGSWTSPPLLLLSTCPGAARAPRLLYSRAAEPLVGQRWEAFDVADAMRRHRREPRPPRAFCLLLRAVAGPVPSPLALRRLGFGWPGGGGSAAEERAVLVVSSRTQRKESLFREIRAQARALGAALASEPLPDPGTGTASPRAVIGGRRRRRTALAGTRTAQGSGGGAGRGHGRRGRSRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR | 65 |
| BMP-13/GDF-6 | MDTPRVLLSAVFLISFLWDLPGFQQASISSSSSSAELGSTKGMRSRKEGKMQRAPRDSDAGREGQEPQPRPQDEPRAQQPRAQEPPGRGPRVVPHEYMLSIYRTYSIAEKLGINASFFQSSKSANTITSFVDRGLDDLSHTPLRRQKYLFDVSMLSDKEELVGAELRLFRQAPSAPWGPPAGPLHVQLFPCLSPLLLDARTLDPQGAPPAGWEVFDVWQGLRHQPWKQLCLELRAAWGELDAGEAEARAR GPQQPPPPDLRSLGFGRRVRPPQERALLVVFTRSQRKNLFAEMREQLGSAEAAGPGAGAEGSWPPPSGAPDARPWLPSPGRRRRRTAFASRHGKRHGKKSRLRCSKKPLHVNFKELGWDDWIIA | 66 |
| | PLEYEAYHCEGVCDFPLRSHLEPTNHAIIQTLMNSMDPGSTPPSCCVPTKLTPISILYIDAGNNVVYKQYEDMVVESCGCR | |
| BMP-15 | MVLLSILRILFLCELVLFMEHRAQMAEGGQSSIALLAEAPTLPLIEELLEESPGEQPRKPRLLGHSLRYMLELYRRSADSHGHPRENRTIGATMVRLVKPLTNVARPHRGTWHIQILGFPLRPNRGLYQLVRATVVYRHHLQLTRFNLSCHVEPWVQKNPTNHFPSSEGDSSKPSLMSNAWKEMDITQLVQQRFWNNKGHRILRLRFMCQQQKDSGGLELWHGTSSLDIAFLLLYFNDTHKSIRKAKFLPRGMEEFMERESLLRRTRQADGISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR | 67 |
| DPP | MKIITYFCIWAVAWAIPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAWQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQGDDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSNSSDSSDSSDSSNSSESSDSSDSSDSDSSDSSDSSNSNSSDSDSSNSSDSSDSSNSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSS | 68 |
| | DSSDSSDGSDSDSSNRSDSSNSSDSSDSSDSSNSSDSSDSSDSNESSNSSDSSDSSNSSDSDSSDSSNSSDSSDSSNSSDSSESSNSSDNSNSSDSSNSSDSSDSSDSSNSSDSSNSSDSSNSSDSSDSNSSDSSDSSNSSDSSDSSDSSDSSDSSDSSNSSDSSDSSDSSDSSNSSDSSNSSDSSNSSDSSDSSDSSDSSDSSDSSDSSDSSNSSDSSDSSDSSDSSDSSDSSDSSDSSESSDSSDSSNSSDSSDSSDSSDSSDSSDSSDSSDSSDSSNSSDSSDSSDSSDSSDSSNSSDSSDSSESSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD | |
| GDF-1 | MPPPQQGPCGHHLLLLLALLLPSLPLTRAPVPPGPAAALLQALGLRDEPQGAPRLRPVPPVMWRLFRRRDPQETRSGSRRTSPGVTLQPCHVEELGVAGNIVRHIPDRGAPTRASEPASAAGHCPEWTVVFDLSAVEPAERPSRARLELRFAAAAAAAPEGGWELSVAQAGQGAGADPGPVLLRQLVPALGPPVRAELLGAAWARNASWPRSLRLALALRPRAPAACARLAEASLLLVTLDPRLCHPLARPRRDAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMWDECGCR | 69 |
| GDF-3 | MLRFLPDLAFSFLLILALGQAVQFQEYVFLQFLGLDKAPSPQKFQPVPYILKKIFQDREAAATTGVSRDLCYVKELGVRGNVLRFLPDQGFFLYPKKISQASSCLQKLLYFNLSAIKEREQLTLAQLGLDLGPNSYYNLGPELELALFLVQEPHVWGQTTPKPGKMFVLRSVPWPQGAVHFNLLDVAKDWNDNPRKNFGLFLEILVKEDRDSGVNFQPEDTCARLRCSLHASLLVVTLNPDQCHPSRKRRAAIPVPKLSCKNLCHRHQLFINFRDLGWHKWIIAPKGFMANYCHGECPFSLTISLNSSNYAFMQALMHAVDPEIPQAVCIPTKLSPISMLYQDNNDNVILRHYEDMVVDECGCG | 70 |
| GDF-5 | MRLPKLLTFLLWYLAWLDLEFICTVLGAPDLGQRPQGTRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAAPGGGRAAQLKLSSCPSGRQPAALLDVRSVPGLDGSGWEVFDIWKLFRNFKNSAQLCLELEAWERGRAVDLRGLGFDRAARQVHEKALFLVFGRTKKRDLFFNEIKARSGQDDKTVYEYLFSQRRKRRAPLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR | 71 |
| GDF-7 | MDLSAAAALCLWLLSACRPRDGLEAAAVLRAAGAGPVRSPGGGGGGGGGGRTLAQAAGAAAVPAAAVPRARAARRAAGSGFRNGSVVPHHFMMSLYRSLAGRAPAGAAAVSASGHGRADTITGFTDQATQDESAAETGQSFLFDVSSLNDADEVVGAELRVLRRGSPESGPGSWTSPPLLLLSTCPGAARAPRLLYSRAAEPLVGQRWEAFDVADAMRRHRREPRPPRAFCLLLRAVAGPVPSPLALRRGFGWPGGGGSAAEERAVLVVSSRTQRKESLFREIRAQARALGAALASEPLPDPGTGTASPRAVIGGRRRRRTALAGTRTAQGSGGGAGRGHGRRGRSRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR | 72 |
| GDF8 | MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFG | 73 |
| | WDWIIAPKRYKANYCSGECEFVFLQKYPHTHLVHQANPRGSAGPCCTPTKMSPINMLYFNGKEQIIYGKIPAMVVDRCGCS | |
| GDF15 | MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI | 74 |
| VEGF | MTDRQTDTAPSPSYBLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR | 75 |
| HABP | MFARMSDLHVLLLMALVGKTACGFSLMSLLESLDPDWTPDQYDYSYEDYNQEENTSSTLTHAENPDWYYTEDQADPCQPNPCEHGGDCLVHGSTFTCSCLAPFSGNKCQKVQNTCKDNPCGRGQCLITQSPPYYRCVCKHPYTGPSCSQVVPVCRPNPCQNGATCSRHKRRSKFTCACPDQFKGKFCEIGSDDCYVGDGYSYRGKMNRTVNQHACLYWNSHLLLQENYNMFMEDAETHGIGEHNFCRNPDADEKPWCFIKVTNDKVKWEYCDVSACSAQDVAYPEESPTEPSTKLPGFDSCGKTEIAERKIKRIYGGFKSTAGKHPWQASLQSSLPLTISMPQGHFCGGALIHPCWVLTAAHCTDIKTRHLKVVLGDQDLKKEEFHEQSFRVEKIFKYSHYNERDEIPHNDI | 76 |
| | ALLKLKPVDGHCALESKYVKTVCLPDGSFPSGSECHISGWGVTETGKGSRQLLDAKVKLIANTLCNSRQLYDHMIDDSMICAGNLQKPGQDTCQGDSGGPLTCEKDGTYYVYGIVSWGLECGKRPGVYTQVTKFLNWIKATIKSESGF | |
| CBP | MRSLLLLSAFCLLEAALAAEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL | 77 |
| FGF-18 | MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA | 78 |
| KGF | MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT | 79 |
| TNFα | MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLL | 80 |
| | SAIKSPCQRETPEGAEAKPWYEPIYLGGVFQLEKGDRLSAEINRPDYL DFAESGQVYFGIIAL | |
| TRAIL | MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFEL KENDRIFVSVTNEHLIDMDHEASFFGAFLVG | 81 |
| WNT1 | MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL | 82 |
| WNT2 | MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT | 83 |
| WNT2B | MLRPGGAEEAAQLPLRRASAPVPVPSPAAPDGSRASARLGLACLLLLLLLTLPARVDTSWWYIGALGARVICDNIPGLVSRQRQLCQRYPDIMRSVGEGAEWIRECQHQFRHHRWNCTTLDRDHTVFGRVMLRSSREAAFVYAISSAGVVHAITRACSQGELSV | 84 |
| | CSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT | |
| WNT3 | MEPHLLGLLLGLLLGGTRVLAGYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK | 85 |
| WNT3A | MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK | 86 |
| WNT4 | MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVL | 87 |
| | GTRGRTCNKTSKAIDGCELLCCGRGFHTAQVELAERCSCKFHWCCFVKCRQCQRLVELHTCR | |
| WNT5A | MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK | 88 |
| WNT5B | MPSLLLLFTAALLSSWAQLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK | 89 |
| WNT6 | MLPPLPSRLGLLLLLLLCPAHVGGLWWAVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL | 90 |
| WNT7A | MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK | 91 |
| WNT7B | MHRNFRKWIFYVFLCFGVLYVKLGALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK | 92 |
| WNT8A | MLCCIQCLCLVSPFPTLTPCQGGPHCLIPIHLCLTFSLFGRSVNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVTMSNPAVLLGIRRIEAKNERVLFRLLKSSLWLQIYADK | 93 |
| WNT8B | MFLSKPSVYICLFTCVLQLSHSWSVNNFLMTGPKAYLIYSSSVAAGAQSGIEECKYQFAWDRWNCPERALQLSSHGGLRSANRETAFVHAISSAGVMYTLTRNCSLGDFDNCGCDDSRNGQLG | 94 |
| | GQGWLWGGCSDNVGFGEAISKQFVDALETGQDARAAMNLHNNEAGRKAVKGTMKRTCKCHGVSGSCTTQTCWLQLPEFREVGAHLKEKYHAALKVDLLQGAGNSAAGRGAIADTFRSISTRELVHLEDSPDYCLENKTLGLLGTEGRECLRRGRALGRWERRSCRRLCGDCGLAVEERRAETVSSCNCKFHWCCAVRCEQCRRRVTKYFCSRAERPRGGAAHKPGRKP | |
| WNT9A | MLDGSPLARWLAAAFGLTLLLAALRPSAAYFGLTGSEPLTILPLTLEPEAAAQAHYKACDRLKLERKQRRMCRRDPGVAETLVEAVSMSALECQFQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETALKVGSTTNEAAGEAGAISPPRGRASGAGGSDPLPRTPELVHLDDSPSFCLAGRFSPGTAGRRCHREKNCESICCGRGHNTQSRVVTRPCQCQVRWCCYVECRQCTQREEVYTCKG | 95 |
| WNT9B | MRPPPALALAGLCLLALPAAAASYFGLTGREVLTPFPGLGTAAAPAQGGAHLKQCDLLKLSRRQKQLCRREPGLAETLRDAAHLGLLECQFQFRHERWNCSLEGRMGLLKRGFKETAFLYAVSSAALTHTLARACSAGRMERCTCDDSPGLESRQAWQWGVCGDNLKYSTKFLSNFLGSKRGNKDLRARADAHNTHVGIKAVKSGLRTTCKCHGVSGSCAVRTCWKQLSPFRETGQVLKLRYDSAVKVSSATNEALGRLELWAPARQGSLTKGLAPRSGDLVYMEDSPSFCRPSKYSPGTAGRVCSREASCSSLCCGRGYDTQSRLVAFSCHCQVQWCCYVECQQCVQEELVYTCKH | 96 |
| WNT10A | MGSAHPRPWLRLRPQPQPRPALWVLLFFLLLLAAAMPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGA | 97 |
| | LLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK | |
| WNT10B | MLEEPRPRPPPSGLAGLLFLALCSRALSNEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK | 98 |
| WNT 11 | MRARPQVCEALLFALALQTGVCYGIKWLALSKTPSALALNQTQHCKQLEGLVSAQVQLCRSNLELMHTVVHAAREVMKACRRAFADMRWNCSSIELAPNYLLDLERGTRESAFVYALSAAAISHAIARACTSGDLPGCSCGPVPGEPPGPGNRWGGCADNLSYGLLMGAKFSDAPMKVKKTGSQANKLMRLHNSEVGRQALRASLEMKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK | 99 |
| WNT16 | MDRAALLGLARLCALWAALLVLFPYGAQGNWMWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK | 100 |

In some embodiments, the mammalian growth factor is a secreted human growth factor. In some embodiments, the mammalian growth factor is an active fragment (e.g., a fragment of a secreted human growth factor, e.g., any of the exemplary growth factors described herein) that has osteogenic activity). Non-limiting in vitro assays to determine whether a fragment of a secreted human growth factor, e.g., any of the exemplary growth factors described herein, has osteogenic activity are described in Kim et al., Amino Acids 42:1455-1465, 2012; Lee et al., ACSMed. Chem. Lett. 2(3):248-251, 2011; and Wang et al., Genetics Mol. Res. 13(2):4456-4465, 2014.

In some embodiments, a fragment of a secreted human growth factor having osteogenic activity can have 1 amino to about 40 amino acids (e.g., 1 amino acid to about 38 amino acids, 1 amino acid to about 36 amino acids, 1 amino acid to about 34 amino acids, 1 amino acid to about 32 amino acids, 1 amino acid to about 30 amino acids, 1 amino acid to about 28 amino acids, 1 amino acid to about 26 amino acid, 1 amino acid to about 24 amino acids, 1 amino acid to about 22 amino acids, 1 amino acid to about 20 amino acids, 1 amino acid to about 18 amino acids, 1 amino acid to about 16 amino acids, 1 amino acid to about 14 amino acids, 1 amino acid to about 12 amino acids, 1 amino acid to about 10 amino acids, 1 amino acid to about 8 amino acids, 1 amino acid to about 6 amino acids, 1 amino acid to about 4 amino acids, or about 1 amino acid to about 3 amino acids) removed from the N-terminus of the secreted human growth factor and/or 1 amino acid to about 20 amino acids (e.g., 1 amino acid to about 18 amino acids, 1 amino acid to about 16 amino acids, 1 amino acid to about 14 amino acids, 1 amino acid to about 12 amino acids, 1 amino acid to about 10 amino acids, 1 amino acid to about 8 amino acids, 1 amino acid to about 6 amino acids, 1 amino acid to about 4 amino acids, or about 1 amino acid to about 3 amino acids) removed from the C-terminus of the secreted human growth factor.

In some embodiments, the mammalian growth factor can have a sequence that is at least 70% identical (e.g., at least 75% identical, at least 80% identical, at least 85% identical, at least 90% identical, at least 95% identical, or at least 99% identical) to any of the sequences in Table B or any secreted human growth factor, and has osteogenic activity. As one skilled in the art can appreciate, the amino acids in a mammalian growth factor that are conserved between different species are likely important for osteogenic activity and should not be mutated, while amino acids in a mammalian growth factor that are not conserved between different species are not likely important for osteogenic activity and can be mutated.

### β-TCP-Binding Polypeptides

Also provided herein are β-tricalcium phosphate (β-TCP)-binding polypeptides that include at least one (e.g., two, three, four, five, six, seven, eight, nine, ten, eleven, twelve, thirteen, fourteen, fifteen, sixteen, seventeen, eighteen, nineteen, or twenty) sequence selected from the group consisting of: VIGESTHHRPWS (SEQ ID NO: 2), LIADSTHHSPWT (SEQ ID NO: 3), ILAESTHHKPWT (SEQ ID NO: 4), ILAETTHHRPWS (SEQ ID NO: 5), IIGESSHHKPFT (SEQ ID NO: 6), GLGDTTHHRPWG (SEQ ID NO: 7), VLGDTTHHKPWT (SEQ ID NO: 8), IVADSTHHRPWT (SEQ ID NO: 9); STADTSHHRPS (SEQ ID NO: 10), TSGGESTHHRPS (SEQ ID NO: 11), TSGGESSHHKPS (SEQ ID NO: 12), TGSGDSSHHRPS (SEQ ID NO: 13), GSSGESTHHKPST (SEQ ID NO: 14), VGADSTHHRPVT (SEQ ID NO: 15), GAADTTHHRPVT (SEQ ID NO: 16), AGADTTHHRPVT (SEQ ID NO: 17), GGADTTHHRPAT (SEQ ID NO: 18), and GGADTTHHRPGT (SEQ ID NO: 19).

Also provided herein are β-TCP-binding polypeptides that include an amino acid sequence of Formula I:
A₀B₀C₀D₀E₀F₀G₀H₀I₀J₀K₀L₀ (Formula I) (SEQ ID NO: 35), where:
   A₀ is V, L, I, G, S, T, or A;
   B₀ is I, L, V, Q, T, S, G, or A;
   C₀ is G, A, V, or S;
   D₀ is E, D, L, or G;
   E₀ is S, T, P T, E, or D;
   F₀ is TorS;
   G₀ is H, T, or S;
   H₀ is H or T;
   I₀ is R, S, K, P, or H;
   J₀ is P, S, R, or K;
   K₀ is W, F, S, P, V, A, or G; and
   L₀ is absent or is S, T, G, (or A);
wherein Formula I does not include LLADTTHHRPWT (SEQ ID NO: 1).

In some embodiments, any of the β-TCP-binding polypeptides described herein can include two or more (e.g., three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more; or 2, 3, 4, 5, 6, 7, 8, 9, 10, 11, 12, 13, 14, 15, 16, 17, 18, 19, 20, 21, or 22) of any of SEQ ID NOs: 2-19 and a sequence of Formula I (not including SEQ ID NO: 1). In some embodiments, each neighboring pair of SEQ ID NOs: 2-19 and a sequence of Formula I (not including SEQ ID NO: 1) directly abut each other. In some embodiments, each neighboring pair of any of SEQ ID NOs: 2-19 and a sequence of Formula I (not including SEQ ID NO: 1) are separated by a linker sequence.

In some embodiments, the chimeric polypeptides can include at least two (e.g., at least three, at least four, at least five, at least six, at least seven, at least eight, at least nine, or at least ten; or 2, 3, 4, 5, 6, 7, 8, 9, or 10) different sequences selected from SEQ ID NOs: 2-19 and a sequence of Formula I (not including SEQ ID NO: 1).

In some embodiments, the β-TCP-binding polypeptide includes LLADTTHHRPWT (SEQ ID NO: 1) or GQVLPTTTPSSP (SEQ ID NO: 19). In some embodiments, the β-TCP-binding polypeptide includes LLADTTHHRPWT (SEQ ID NO: 1) and VIGESTHHRPWS (SEQ ID NO: 2). In some embodiments, the β-TCP-binding polypeptide includes the sequence of LLADTTHHRPWTVIGESTHHRPWS (SEQ ID NO: 20).

In some embodiments, the β-TCP-binding polypeptide includes LLADTTHHRPWT (SEQ ID NO: 1), VIGESTHHRPWS (SEQ ID NO: 2), and IIGESSHHKPFT (SEQ ID NO: 6). In some embodiments, the β-TCP-binding polypeptide includes the sequence of LLADTTHHRPWTVIGESTHHRPWSIIGESSHHKPFT (SEQ ID NO: 21).

In some embodiments, the β-TCP-binding polypeptide includes LLADTTHHRPWT (SEQ ID NO: 1), VIGESTHHRPWS (SEQ ID NO: 2), IIGESSHHKPFT (SEQ ID NO: 6), GLGDTTHHRPWG (SEQ ID NO: 7), and ILAESTHHKPWT (SEQ ID NO: 4). In some embodiments, the β-TCP-binding polypeptide includes the sequence of: LLADTTHHRPWTVIGESTHHRPWSIIGESSHHKPFTGLGDTTHHRPWGILAESTHHKP WT (SEQ ID NO: 22).

In some embodiments, the β-TCP-binding polypeptide includes LLADTTHHRPWT (SEQ ID NO: 1) and ILAESTHHKPWT (SEQ ID NO: 4). In some embodiments, the β-TCP-binding polypeptide includes at least one copy of the sequence LLADTTHHRPWTILAESTHHKPWT (SEQ ID NO: 23). In some embodiments, the β-TCP-binding polypeptide includes the sequence of LLADTTHHRPWTILAESTHHKPWTLLADTTHHRPWTILAESTHHKPWTLLADTTHHRPWT (SEQ ID NO: 24).

In some embodiments, the β-TCP-binding polypeptide includes LLADTTHHRPWT (SEQ ID NO: 1) and GLGDTTHHRPWG (SEQ ID NO: 7). In some embodiments, the β-TCP-binding polypeptide includes at least one copy of the sequence of LLADTTHHRPWTGLGDTTHHRPWG (SEQ ID NO: 25). In some embodiments, the β-TCP-binding polypeptide includes the sequence of LLADTTHHRPWTGLGDTTHHRPWGLLADTTHHRPWT (SEQ ID NO: 26). In some embodiments, the β-TCP-binding polypeptide includes the sequence of LLADTTHHRPWTGLGDTTHHRPWGLLADTTHHRPWTGLGDTTHHRPWGLLADTTH HRPWT (SEQ ID NO: 27). In some embodiments, the β-TCP-binding polypeptide includes the sequence of LLADTTHHRPWTGLGDTTHHRPWGLLADTTHHRPWTGLGDTTHHRPWGLLADTTHHRPWTGLGDTTHHRPWGLLADTTHHRPWT (SEQ ID NO: 28).

In some embodiments, the β-TCP-binding polypeptide includes STADTSHHRPS (SEQ ID NO: 10), TSGGESTHHRPS (SEQ ID NO: 11), TSGGESSHHKPS (SEQ ID NO: 12), TGSGDSSHHRPS (SEQ ID NO: 13), and GSSGESTHHKPST (SEQ ID NO: 14). In some embodiments, the β-TCP-binding polypeptide includes the sequence of STADTSHHRPSTSGGESTHHRPSTSGGESSHHKPSTGSGDSSHHRPSGSSGESTHHKPST (SEQ ID NO: 29).

In some embodiments, the β-TCP-binding polypeptide includes VGADSTHHRPVT (SEQ ID NO: 15), GAADTTHHRPVT SEQ ID NO: 16), AGADTTHHRPVT (SEQ ID NO: 17), GGADTTHERPAT (SEQ ID NO: 18) and GGADTTHHRPGT (SEQ ID NO: 19). In some embodiments, the β-TCP-binding polypeptide includes the sequence of VGADSTHHRPVTGAADTTHHRPVTAGADTTHHRPVTGGADTTHHRPATGGADTTH HRPGT (SEQ ID NO: 30).

In some embodiments, the β-TCP-binding polypeptide includes STADTSHHRPS (SEQ ID NO: 10), LLADTTHHRPWT (SEQ ID NO: 1), TSGGESTHHRPS (SEQ ID NO: 11), VGADSTHHRPVT (SEQ ID NO: 15), TSGGESSHHKPS (SEQ ID NO: 12), GAADTTHHRPVT (SEQ ID NO: 16), TGSGDSSHHRPS (SEQ ID NO: 13), GSSGESTHHKPS (SEQ ID NO: 14), and TGGADTTHHRPAT (SEQ ID NO: 18). In some embodiments, the β-TCP-binding polypeptide includes the sequence of: STADTSHHRPSLLADTTHHRPWTTSGGESTHHRPSVGADSTHHRPVTTSGGESSHHKPSGAADTTHHRPVTTGSGDSSHHRPSGSSGESTHHKPSTGGADTTHHRPAT (SEQ ID NO: 31).

In some embodiments, the β-TCP-binding polypeptides can include two or more (e.g., three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more; or 2, 3, 4, 5, 6, 7, 8, 9, 10, 11, 12, 13, 14, 15, 16, 17, 18, 19, or 20) copies of any one of SEQ ID NOs: 2-19 or a sequence of Formula I (excluding SEQ ID NO: 1).

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 1 (but excluding SEQ ID NO: 1).

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 2.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 3.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 4.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 5.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 6.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 7.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 8.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 9.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 10.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 11.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 12.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 13.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 14.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 15.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 16.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 17.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 18.

In some embodiments, the β-TCP-binding polypeptides can include an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 19.

In some embodiments, the β-TCP-binding polypeptide further includes a linker sequence (e.g., any of the exemplary linker sequences described herein or known in the art) between a neighboring pair of any of SEQ ID NOs. 2-19 and a sequence of Formula I (excluding SEQ ID NO: 1). In some embodiments, the linker sequence can include the sequence of TGGSGEGGTGASTGGSAGTGGSGGTTSGEAGGSSGAG (SEQ ID NO: 33). In some embodiments, the linker sequence consists of TGGSGEGGTGASTGGSAGTGGSGGTTSGEAGGSSGAG (SEQ ID NO: 33). In some embodiments, the linker sequence includes the sequence of GSGATG (SEQ ID NO: 34). In some embodiments, the linker sequence consists of GSGATG (SEQ ID NO: 34).

In some embodiments, where the β-TCP-binding polypeptide includes two or more or any of SEQ ID Nos. 2-19 and a sequence of Formula I (excluding SEQ ID NO: 1), a linker sequence (e.g., any of the exemplary linker sequences described herein or known in the art) can be between any of the pairs (e.g., all of the pairs) of neighboring sequences selected from the group of SEQ ID NOs: 2-19 and a sequence of Formula I (excluding SEQ ID NO: 1).

In some embodiments, the β-TCP-binding polypeptide can include a signal sequence at its N-terminus. In some embodiments, the β-TCP-binding polypeptide can further include a tag sequence (e.g., a poly-His tag, chitin-binding protein (CBP), maltose-binding protein (MBP), strep-tag, glutathione-S-transferase (GST), thioredoxin, or Fc region). Additional examples of tags are known in the art.

The β-TCP-binding polypeptides described herein can bind to β-TCP (e.g., any of the types of β-TCP described herein) with a dissociation equilibrium constant (K_{d}) of about 1 pM to about 100 µM (or any of the subranges of this range described herein) (e.g., as measured using SPR in phosphate buffered saline).

In some embodiments, the β-TCP-binding polypeptides described herein can have a total length of about 11 amino acids to about 1400 amino acids, about 11 amino acids to about 1350 amino acids, about 11 amino acids to about 1300 amino acids, about 11 amino acids to about 1250 amino acids, about 11 amino acids to about 1200 amino acids, about 11 amino acids to about 1150 amino acids, about 11 amino acids to about 1100 amino acids, about 11 amino acid to about 1050 amino acids, about 11 amino acids to about 1000 amino acids, about 11 amino acids to about 950 amino acids, about 11 amino acids to about 900 amino acids, about 11 amino acids to about 850 amino acids, about 11 amino acids to about 800 amino acids, about 11 amino acids to about 750 amino acids, about 11 amino acids to about 700 amino acids, about 11 amino acids to about 650 amino acids, about 11 amino acids to about 600 amino acids, about 11 amino acids to about 550 amino acids, about 11 amino acids to about 500 amino acids, about 11 amino acids to about 450 amino acids, about 11 amino acids to about 400 amino acids, about 11 amino acids to about 350 amino acids, about 11 amino acids to about 300 amino acids, about 11 amino acids to about 250 amino acids, about 11 amino acids to about 200 amino acids, about 11 amino acids to about 150 amino acids, about 11 amino acids to about 100 amino acids, about 11 amino acids to about 90 amino acids, about 11 amino acids to about 80 amino acids, about 11 amino acids to about 70 amino acids, about 11 amino acids to about 60 amino acids, about 11 amino acids to about 50 amino acids, about 11 amino acids to about 45 amino acids, about 11 amino acids to about 40 amino acids, about 11 amino acids to about 35 amino acids, about 11 amino acids to about 30 amino acids, about 11 amino acids to about 25 amino acids, about 11 amino acids to about 20 amino acids, about 11 amino acids to about 18 amino acids, about 11 amino acids to about 16 amino acids, about 11 amino acids to about 14 amino acids, about 12 amino acids to about 1400 amino acids, about 12 amino acids to about 1350 amino acids, about 12 amino acids to about 1300 amino acids, about 12 amino acids to about 1250 amino acids, about 12 amino acids to about 1200 amino acids, about 12 amino acids to about 1150 amino acids, about 12 amino acids to about 1100 amino acids, about 12 amino acid to about 1050 amino acids, about 12 amino acids to about 1000 amino acids, about 12 amino acids to about 950 amino acids, about 12 amino acids to about 900 amino acids, about 12 amino acids to about 850 amino acids, about 12 amino acids to about 800 amino acids, about 12 amino acids to about 750 amino acids, about 12 amino acids to about 700 amino acids, about 12 amino acids to about 650 amino acids, about 12 amino acids to about 600 amino acids, about 12 amino acids to about 550 amino acids, about 12 amino acids to about 500 amino acids, about 12 amino acids to about 450 amino acids, about 12 amino acids to about 400 amino acids, about 12 amino acids to about 350 amino acids, about 12 amino acids to about 300 amino acids, about 12 amino acids to about 250 amino acids, about 12 amino acids to about 200 amino acids, about 12 amino acids to about 150 amino acids, about 12 amino acids to about 100 amino acids, about 12 amino acids to about 90 amino acids, about 12 amino acids to about 80 amino acids, about 12 amino acids to about 70 amino acids, about 12 amino acids to about 60 amino acids, about 12 amino acids to about 50 amino acids, about 12 amino acids to about 45 amino acids, about 12 amino acids to about 40 amino acids, about 12 amino acids to about 35 amino acids, about 12 amino acids to about 30 amino acids, about 12 amino acids to about 25 amino acids, about 12 amino acids to about 20 amino acids, about 12 amino acids to about 18 amino acids, about 12 amino acids to about 16 amino acids, about 12 amino acids to about 14 amino acids, about 14 amino acids to about 1400 amino acids, about 14 amino acids to about 1350 amino acids, about 14 amino acids to about 1300 amino acids, about 14 amino acids to about 1250 amino acids, about 14 amino acids to about 1200 amino acids, about 14 amino acids to about 1150 amino acids, about 14 amino acids to about 1100 amino acids, about 14 amino acid to about 1050 amino acids, about 14 amino acids to about 1000 amino acids, about 14 amino acids to about 950 amino acids, about 14 amino acids to about 900 amino acids, about 14 amino acids to about 850 amino acids, about 14 amino acids to about 800 amino acids, about 14 amino acids to about 750 amino acids, about 14 amino acids to about 700 amino acids, about 14 amino acids to about 650 amino acids, about 14 amino acids to about 600 amino acids, about 14 amino acids to about 550 amino acids, about 14 amino acids to about 500 amino acids, about 14 amino acids to about 450 amino acids, about 14 amino acids to about 400 amino acids, about 14 amino acids to about 350 amino acids, about 14 amino acids to about 300 amino acids, about 14 amino acids to about 250 amino acids, about 14 amino acids to about 200 amino acids, about 14 amino acids to about 150 amino acids, about 14 amino acids to about 100 amino acids, about 14 amino acids to about 90 amino acids, about 14 amino acids to about 80 amino acids, about 14 amino acids to about 70 amino acids, about 14 amino acids to about 60 amino acids, about 14 amino acids to about 50 amino acids, about 14 amino acids to about 45 amino acids, about 14 amino acids to about 40 amino acids, about 14 amino acids to about 35 amino acids, about 14 amino acids to about 30 amino acids, about 14 amino acids to about 25 amino acids, about 14 amino acids to about 20 amino acids, about 14 amino acids to about 18 amino acids, about 14 amino acids to about 16 amino acids, about 16 amino acids to about 1400 amino acids, about 16 amino acids to about 1350 amino acids, about 16 amino acids to about 1300 amino acids, about 16 amino acids to about 1250 amino acids, about 16 amino acids to about 1200 amino acids, about 16 amino acids to about 1150 amino acids, about 16 amino acids to about 1100 amino acids, about 16 amino acid to about 1050 amino acids, about 16 amino acids to about 1000 amino acids, about 16 amino acids to about 950 amino acids, about 16 amino acids to about 900 amino acids, about 16 amino acids to about 850 amino acids, about 16 amino acids to about 800 amino acids, about 16 amino acids to about 750 amino acids, about 16 amino acids to about 700 amino acids, about 16 amino acids to about 650 amino acids, about 16 amino acids to about 600 amino acids, about 16 amino acids to about 550 amino acids, about 16 amino acids to about 500 amino acids, about 16 amino acids to about 450 amino acids, about 16 amino acids to about 400 amino acids, about 16 amino acids to about 350 amino acids, about 16 amino acids to about 300 amino acids, about 16 amino acids to about 250 amino acids, about 16 amino acids to about 200 amino acids, about 16 amino acids to about 150 amino acids, about 16 amino acids to about 100 amino acids, about 16 amino acids to about 90 amino acids, about 16 amino acids to about 80 amino acids, about 16 amino acids to about 70 amino acids, about 16 amino acids to about 60 amino acids, about 16 amino acids to about 50 amino acids, about 16 amino acids to about 45 amino acids, about 16 amino acids to about 40 amino acids, about 16 amino acids to about 35 amino acids, about 16 amino acids to about 30 amino acids, about 16 amino acids to about 25 amino acids, about 16 amino acids to about 20 amino acids, about 16 amino acids to about 18 amino acids, about 18 amino acids to about 1400 amino acids, about 18 amino acids to about 1350 amino acids, about 18 amino acids to about 1300 amino acids, about 18 amino acids to about 1250 amino acids, about 18 amino acids to about 1200 amino acids, about 18 amino acids to about 1150 amino acids, about 18 amino acids to about 1100 amino acids, about 18 amino acid to about 1050 amino acids, about 18 amino acids to about 1000 amino acids, about 18 amino acids to about 950 amino acids, about 18 amino acids to about 900 amino acids, about 18 amino acids to about 850 amino acids, about 18 amino acids to about 800 amino acids, about 18 amino acids to about 750 amino acids, about 18 amino acids to about 700 amino acids, about 18 amino acids to about 650 amino acids, about 18 amino acids to about 600 amino acids, about 18 amino acids to about 550 amino acids, about 18 amino acids to about 500 amino acids, about 18 amino acids to about 450 amino acids, about 18 amino acids to about 400 amino acids, about 18 amino acids to about 350 amino acids, about 18 amino acids to about 300 amino acids, about 18 amino acids to about 250 amino acids, about 18 amino acids to about 200 amino acids, about 18 amino acids to about 150 amino acids, about 18 amino acids to about 100 amino acids, about 18 amino acids to about 90 amino acids, about 18 amino acids to about 80 amino acids, about 18 amino acids to about 70 amino acids, about 18 amino acids to about 60 amino acids, about 18 amino acids to about 50 amino acids, about 18 amino acids to about 45 amino acids, about 18 amino acids to about 40 amino acids, about 18 amino acids to about 35 amino acids, about 18 amino acids to about 30 amino acids, about 18 amino acids to about 25 amino acids, about 18 amino acids to about 20 amino acids, about 20 amino acids to about 1400 amino acids, about 20 amino acids to about 1350 amino acids, about 20 amino acids to about 1300 amino acids, about 20 amino acids to about 1250 amino acids, about 20 amino acids to about 1200 amino acids, about 20 amino acids to about 1150 amino acids, about 20 amino acids to about 1100 amino acids, about 20 amino acid to about 1050 amino acids, about 20 amino acids to about 1000 amino acids, about 20 amino acids to about 950 amino acids, about 20 amino acids to about 900 amino acids, about 20 amino acids to about 850 amino acids, about 20 amino acids to about 800 amino acids, about 20 amino acids to about 750 amino acids, about 20 amino acids to about 700 amino acids, about 20 amino acids to about 650 amino acids, about 20 amino acids to about 600 amino acids, about 20 amino acids to about 550 amino acids, about 20 amino acids to about 500 amino acids, about 20 amino acids to about 450 amino acids, about 20 amino acids to about 400 amino acids, about 20 amino acids to about 350 amino acids, about 20 amino acids to about 300 amino acids, about 20 amino acids to about 250 amino acids, about 20 amino acids to about 200 amino acids, about 20 amino acids to about 150 amino acids, about 20 amino acids to about 100 amino acids, about 20 amino acids to about 90 amino acids, about 20 amino acids to about 80 amino acids, about 20 amino acids to about 70 amino acids, about 20 amino acids to about 60 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 1400 amino acids, about 25 amino acids to about 1350 amino acids, about 25 amino acids to about 1300 amino acids, about 25 amino acids to about 1250 amino acids, about 25 amino acids to about 1200 amino acids, about 25 amino acids to about 1150 amino acids, about 25 amino acids to about 1100 amino acids, about 25 amino acid to about 1050 amino acids, about 25 amino acids to about 1000 amino acids, about 25 amino acids to about 950 amino acids, about 25 amino acids to about 900 amino acids, about 25 amino acids to about 850 amino acids, about 25 amino acids to about 800 amino acids, about 25 amino acids to about 750 amino acids, about 25 amino acids to about 700 amino acids, about 25 amino acids to about 650 amino acids, about 25 amino acids to about 600 amino acids, about 25 amino acids to about 550 amino acids, about 25 amino acids to about 500 amino acids, about 25 amino acids to about 450 amino acids, about 25 amino acids to about 400 amino acids, about 25 amino acids to about 350 amino acids, about 25 amino acids to about 300 amino acids, about 25 amino acids to about 250 amino acids, about 25 amino acids to about 200 amino acids, about 25 amino acids to about 150 amino acids, about 25 amino acids to about 100 amino acids, about 25 amino acids to about 90 amino acids, about 25 amino acids to about 80 amino acids, about 25 amino acids to about 70 amino acids, about 25 amino acids to about 60 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 1400 amino acids, about 30 amino acids to about 1350 amino acids, about 30 amino acids to about 1300 amino acids, about 30 amino acids to about 1250 amino acids, about 30 amino acids to about 1200 amino acids, about 30 amino acids to about 1150 amino acids, about 30 amino acids to about 1100 amino acids, about 30 amino acid to about 1050 amino acids, about 30 amino acids to about 1000 amino acids, about 30 amino acids to about 950 amino acids, about 30 amino acids to about 900 amino acids, about 30 amino acids to about 850 amino acids, about 30 amino acids to about 800 amino acids, about 30 amino acids to about 750 amino acids, about 30 amino acids to about 700 amino acids, about 30 amino acids to about 650 amino acids, about 30 amino acids to about 600 amino acids, about 30 amino acids to about 550 amino acids, about 30 amino acids to about 500 amino acids, about 30 amino acids to about 450 amino acids, about 30 amino acids to about 400 amino acids, about 30 amino acids to about 350 amino acids, about 30 amino acids to about 300 amino acids, about 30 amino acids to about 250 amino acids, about 30 amino acids to about 200 amino acids, about 30 amino acids to about 150 amino acids, about 30 amino acids to about 100 amino acids, about 30 amino acids to about 90 amino acids, about 30 amino acids to about 80 amino acids, about 30 amino acids to about 70 amino acids, about 30 amino acids to about 60 amino acids, about 30 amino acids to about 50 amino acids, about 30 amino acids to about 45 amino acids, about 30 amino acids to about 40 amino acids, about 30 amino acids to about 35 amino acids, about 35 amino acids to about 1400 amino acids, about 35 amino acids to about 1350 amino acids, about 35 amino acids to about 1300 amino acids, about 35 amino acids to about 1250 amino acids, about 35 amino acids to about 1200 amino acids, about 35 amino acids to about 1150 amino acids, about 35 amino acids to about 1100 amino acids, about 35 amino acid to about 1050 amino acids, about 35 amino acids to about 1000 amino acids, about 35 amino acids to about 950 amino acids, about 35 amino acids to about 900 amino acids, about 35 amino acids to about 850 amino acids, about 35 amino acids to about 800 amino acids, about 35 amino acids to about 750 amino acids, about 35 amino acids to about 700 amino acids, about 35 amino acids to about 650 amino acids, about 35 amino acids to about 600 amino acids, about 35 amino acids to about 550 amino acids, about 35 amino acids to about 500 amino acids, about 35 amino acids to about 450 amino acids, about 35 amino acids to about 400 amino acids, about 35 amino acids to about 350 amino acids, about 35 amino acids to about 300 amino acids, about 35 amino acids to about 250 amino acids, about 35 amino acids to about 200 amino acids, about 35 amino acids to about 150 amino acids, about 35 amino acids to about 100 amino acids, about 35 amino acids to about 90 amino acids, about 35 amino acids to about 80 amino acids, about 35 amino acids to about 70 amino acids, about 35 amino acids to about 60 amino acids, about 35 amino acids to about 50 amino acids, about 35 amino acids to about 45 amino acids, about 35 amino acids to about 40 amino acids, about 40 amino acids to about 1400 amino acids, about 40 amino acids to about 1350 amino acids, about 40 amino acids to about 1300 amino acids, about 40 amino acids to about 1250 amino acids, about 40 amino acids to about 1200 amino acids, about 40 amino acids to about 1150 amino acids, about 40 amino acids to about 1100 amino acids, about 40 amino acid to about 1050 amino acids, about 40 amino acids to about 1000 amino acids, about 40 amino acids to about 950 amino acids, about 40 amino acids to about 900 amino acids, about 40 amino acids to about 850 amino acids, about 40 amino acids to about 800 amino acids, about 40 amino acids to about 750 amino acids, about 40 amino acids to about 700 amino acids, about 40 amino acids to about 650 amino acids, about 40 amino acids to about 600 amino acids, about 40 amino acids to about 550 amino acids, about 40 amino acids to about 500 amino acids, about 40 amino acids to about 450 amino acids, about 40 amino acids to about 400 amino acids, about 40 amino acids to about 350 amino acids, about 40 amino acids to about 300 amino acids, about 40 amino acids to about 250 amino acids, about 40 amino acids to about 200 amino acids, about 40 amino acids to about 150 amino acids, about 40 amino acids to about 100 amino acids, about 40 amino acids to about 90 amino acids, about 40 amino acids to about 80 amino acids, about 40 amino acids to about 70 amino acids, about 40 amino acids to about 60 amino acids, about 40 amino acids to about 50 amino acids, about 40 amino acids to about 45 amino acids, about 45 amino acids to about 1400 amino acids, about 45 amino acids to about 1350 amino acids, about 45 amino acids to about 1300 amino acids, about 45 amino acids to about 1250 amino acids, about 45 amino acids to about 1200 amino acids, about 45 amino acids to about 1150 amino acids, about 45 amino acids to about 1100 amino acids, about 45 amino acid to about 1050 amino acids, about 45 amino acids to about 1000 amino acids, about 45 amino acids to about 950 amino acids, about 45 amino acids to about 900 amino acids, about 45 amino acids to about 850 amino acids, about 45 amino acids to about 800 amino acids, about 45 amino acids to about 750 amino acids, about 45 amino acids to about 700 amino acids, about 45 amino acids to about 650 amino acids, about 45 amino acids to about 600 amino acids, about 45 amino acids to about 550 amino acids, about 45 amino acids to about 500 amino acids, about 45 amino acids to about 450 amino acids, about 45 amino acids to about 400 amino acids, about 45 amino acids to about 350 amino acids, about 45 amino acids to about 300 amino acids, about 45 amino acids to about 250 amino acids, about 45 amino acids to about 200 amino acids, about 45 amino acids to about 150 amino acids, about 45 amino acids to about 100 amino acids, about 45 amino acids to about 90 amino acids, about 45 amino acids to about 80 amino acids, about 45 amino acids to about 70 amino acids, about 45 amino acids to about 60 amino acids, about 45 amino acids to about 50 amino acids, about 50 amino acids to about 1400 amino acids, about 50 amino acids to about 1350 amino acids, about 50 amino acids to about 1300 amino acids, about 50 amino acids to about 1250 amino acids, about 50 amino acids to about 1200 amino acids, about 50 amino acids to about 1150 amino acids, about 50 amino acids to about 1100 amino acids, about 50 amino acid to about 1050 amino acids, about 50 amino acids to about 1000 amino acids, about 50 amino acids to about 950 amino acids, about 50 amino acids to about 900 amino acids, about 50 amino acids to about 850 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 250 amino acids, about 50 amino acids to about 200 amino acids, about 50 amino acids to about 150 amino acids, about 50 amino acids to about 100 amino acids, about 50 amino acids to about 90 amino acids, about 50 amino acids to about 80 amino acids, about 50 amino acids to about 70 amino acids, about 50 amino acids to about 60 amino acids, about 60 amino acids to about 1400 amino acids, about 60 amino acids to about 1350 amino acids, about 60 amino acids to about 1300 amino acids, about 60 amino acids to about 1250 amino acids, about 60 amino acids to about 1200 amino acids, about 60 amino acids to about 1150 amino acids, about 60 amino acids to about 1100 amino acids, about 60 amino acid to about 1050 amino acids, about 60 amino acids to about 1000 amino acids, about 60 amino acids to about 950 amino acids, about 60 amino acids to about 900 amino acids, about 60 amino acids to about 850 amino acids, about 60 amino acids to about 800 amino acids, about 60 amino acids to about 750 amino acids, about 60 amino acids to about 700 amino acids, about 60 amino acids to about 650 amino acids, about 60 amino acids to about 600 amino acids, about 60 amino acids to about 550 amino acids, about 60 amino acids to about 500 amino acids, about 60 amino acids to about 450 amino acids, about 60 amino acids to about 400 amino acids, about 60 amino acids to about 350 amino acids, about 60 amino acids to about 300 amino acids, about 60 amino acids to about 250 amino acids, about 60 amino acids to about 200 amino acids, about 60 amino acids to about 150 amino acids, about 60 amino acids to about 100 amino acids, about 60 amino acids to about 90 amino acids, about 60 amino acids to about 80 amino acids, about 60 amino acids to about 70 amino acids, about 70 amino acids to about 1400 amino acids, about 70 amino acids to about 1350 amino acids, about 70 amino acids to about 1300 amino acids, about 70 amino acids to about 1250 amino acids, about 70 amino acids to about 1200 amino acids, about 70 amino acids to about 1150 amino acids, about 70 amino acids to about 1100 amino acids, about 70 amino acid to about 1050 amino acids, about 70 amino acids to about 1000 amino acids, about 70 amino acids to about 950 amino acids, about 70 amino acids to about 900 amino acids, about 70 amino acids to about 850 amino acids, about 70 amino acids to about 800 amino acids, about 70 amino acids to about 750 amino acids, about 70 amino acids to about 700 amino acids, about 70 amino acids to about 650 amino acids, about 70 amino acids to about 600 amino acids, about 70 amino acids to about 550 amino acids, about 70 amino acids to about 500 amino acids, about 70 amino acids to about 450 amino acids, about 70 amino acids to about 400 amino acids, about 70 amino acids to about 350 amino acids, about 70 amino acids to about 300 amino acids, about 70 amino acids to about 250 amino acids, about 70 amino acids to about 200 amino acids, about 70 amino acids to about 150 amino acids, about 70 amino acids to about 100 amino acids, about 70 amino acids to about 90 amino acids, about 70 amino acids to about 80 amino acids, about 80 amino acids to about 1400 amino acids, about 80 amino acids to about 1350 amino acids, about 80 amino acids to about 1300 amino acids, about 80 amino acids to about 1250 amino acids, about 80 amino acids to about 1200 amino acids, about 80 amino acids to about 1150 amino acids, about 80 amino acids to about 1100 amino acids, about 80 amino acid to about 1050 amino acids, about 80 amino acids to about 1000 amino acids, about 80 amino acids to about 950 amino acids, about 80 amino acids to about 900 amino acids, about 80 amino acids to about 850 amino acids, about 80 amino acids to about 800 amino acids, about 80 amino acids to about 750 amino acids, about 80 amino acids to about 700 amino acids, about 80 amino acids to about 650 amino acids, about 80 amino acids to about 600 amino acids, about 80 amino acids to about 550 amino acids, about 80 amino acids to about 500 amino acids, about 80 amino acids to about 450 amino acids, about 80 amino acids to about 400 amino acids, about 80 amino acids to about 350 amino acids, about 80 amino acids to about 300 amino acids, about 80 amino acids to about 250 amino acids, about 80 amino acids to about 200 amino acids, about 80 amino acids to about 150 amino acids, about 80 amino acids to about 100 amino acids, about 80 amino acids to about 90 amino acids, about 90 amino acids to about 1400 amino acids, about 90 amino acids to about 1350 amino acids, about 90 amino acids to about 1300 amino acids, about 90 amino acids to about 1250 amino acids, about 90 amino acids to about 1200 amino acids, about 90 amino acids to about 1150 amino acids, about 90 amino acids to about 1100 amino acids, about 90 amino acid to about 1050 amino acids, about 90 amino acids to about 1000 amino acids, about 90 amino acids to about 950 amino acids, about 90 amino acids to about 900 amino acids, about 90 amino acids to about 850 amino acids, about 90 amino acids to about 800 amino acids, about 90 amino acids to about 750 amino acids, about 90 amino acids to about 700 amino acids, about 90 amino acids to about 650 amino acids, about 90 amino acids to about 600 amino acids, about 90 amino acids to about 550 amino acids, about 90 amino acids to about 500 amino acids, about 90 amino acids to about 450 amino acids, about 90 amino acids to about 400 amino acids, about 90 amino acids to about 350 amino acids, about 90 amino acids to about 300 amino acids, about 90 amino acids to about 250 amino acids, about 90 amino acids to about 200 amino acids, about 90 amino acids to about 150 amino acids, about 90 amino acids to about 100 amino acids, about 100 amino acids to about 1400 amino acids, about 100 amino acids to about 1350 amino acids, about 100 amino acids to about 1300 amino acids, about 100 amino acids to about 1250 amino acids, about 100 amino acids to about 1200 amino acids, about 100 amino acids to about 1150 amino acids, about 100 amino acids to about 1100 amino acids, about 100 amino acid to about 1050 amino acids, about 100 amino acids to about 1000 amino acids, about 100 amino acids to about 950 amino acids, about 100 amino acids to about 900 amino acids, about 100 amino acids to about 850 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 250 amino acids, about 100 amino acids to about 200 amino acids, about 100 amino acids to about 150 amino acids, about 150 amino acids to about 1400 amino acids, about 150 amino acids to about 1350 amino acids, about 150 amino acids to about 1300 amino acids, about 150 amino acids to about 1250 amino acids, about 150 amino acids to about 1200 amino acids, about 150 amino acids to about 1150 amino acids, about 150 amino acids to about 1100 amino acids, about 150 amino acid to about 1050 amino acids, about 150 amino acids to about 1000 amino acids, about 150 amino acids to about 950 amino acids, about 150 amino acids to about 900 amino acids, about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 1400 amino acids, about 200 amino acids to about 1350 amino acids, about 200 amino acids to about 1300 amino acids, about 200 amino acids to about 1250 amino acids, about 200 amino acids to about 1200 amino acids, about 200 amino acids to about 1150 amino acids, about 200 amino acids to about 1100 amino acids, about 200 amino acid to about 1050 amino acids, about 200 amino acids to about 1000 amino acids, about 200 amino acids to about 950 amino acids, about 200 amino acids to about 900 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 1400 amino acids, about 250 amino acids to about 1350 amino acids, about 250 amino acids to about 1300 amino acids, about 250 amino acids to about 1250 amino acids, about 250 amino acids to about 1200 amino acids, about 250 amino acids to about 1150 amino acids, about 250 amino acids to about 1100 amino acids, about 250 amino acid to about 1050 amino acids, about 250 amino acids to about 1000 amino acids, about 250 amino acids to about 950 amino acids, about 250 amino acids to about 900 amino acids, about 250 amino acids to about 850 amino acids, about 250 amino acids to about 800 amino acids, about 250 amino acids to about 750 amino acids, about 250 amino acids to about 700 amino acids, about 250 amino acids to about 650 amino acids, about 250 amino acids to about 600 amino acids, about 250 amino acids to about 550 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 1400 amino acids, about 300 amino acids to about 1350 amino acids, about 300 amino acids to about 1300 amino acids, about 300 amino acids to about 1250 amino acids, about 300 amino acids to about 1200 amino acids, about 300 amino acids to about 1150 amino acids, about 300 amino acids to about 1100 amino acids, about 300 amino acid to about 1050 amino acids, about 300 amino acids to about 1000 amino acids, about 300 amino acids to about 950 amino acids, about 300 amino acids to about 900 amino acids, about 300 amino acids to about 850 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 1400 amino acids, about 350 amino acids to about 1350 amino acids, about 350 amino acids to about 1300 amino acids, about 350 amino acids to about 1250 amino acids, about 350 amino acids to about 1200 amino acids, about 350 amino acids to about 1150 amino acids, about 350 amino acids to about 1100 amino acids, about 350 amino acid to about 1050 amino acids, about 350 amino acids to about 1000 amino acids, about 350 amino acids to about 950 amino acids, about 350 amino acids to about 900 amino acids, about 350 amino acids to about 850 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 1400 amino acids, about 400 amino acids to about 1350 amino acids, about 400 amino acids to about 1300 amino acids, about 400 amino acids to about 1250 amino acids, about 400 amino acids to about 1200 amino acids, about 400 amino acids to about 1150 amino acids, about 400 amino acids to about 1100 amino acids, about 400 amino acid to about 1050 amino acids, about 400 amino acids to about 1000 amino acids, about 400 amino acids to about 950 amino acids, about 400 amino acids to about 900 amino acids, about 400 amino acids to about 850 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 1400 amino acids, about 450 amino acids to about 1350 amino acids, about 450 amino acids to about 1300 amino acids, about 450 amino acids to about 1250 amino acids, about 450 amino acids to about 1200 amino acids, about 450 amino acids to about 1150 amino acids, about 450 amino acids to about 1100 amino acids, about 450 amino acid to about 1050 amino acids, about 450 amino acids to about 1000 amino acids, about 450 amino acids to about 950 amino acids, about 450 amino acids to about 900 amino acids, about 450 amino acids to about 850 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 1400 amino acids, about 500 amino acids to about 1350 amino acids, about 500 amino acids to about 1300 amino acids, about 500 amino acids to about 1250 amino acids, about 500 amino acids to about 1200 amino acids, about 500 amino acids to about 1150 amino acids, about 500 amino acids to about 1100 amino acids, about 500 amino acid to about 1050 amino acids, about 500 amino acids to about 1000 amino acids, about 500 amino acids to about 950 amino acids, about 500 amino acids to about 900 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 1400 amino acids, about 550 amino acids to about 1350 amino acids, about 550 amino acids to about 1300 amino acids, about 550 amino acids to about 1250 amino acids, about 550 amino acids to about 1200 amino acids, about 550 amino acids to about 1150 amino acids, about 550 amino acids to about 1100 amino acids, about 550 amino acid to about 1050 amino acids, about 550 amino acids to about 1000 amino acids, about 550 amino acids to about 950 amino acids, about 550 amino acids to about 900 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 1400 amino acids, about 600 amino acids to about 1350 amino acids, about 600 amino acids to about 1300 amino acids, about 600 amino acids to about 1250 amino acids, about 600 amino acids to about 1200 amino acids, about 600 amino acids to about 1150 amino acids, about 600 amino acids to about 1100 amino acids, about 600 amino acid to about 1050 amino acids, about 600 amino acids to about 1000 amino acids, about 600 amino acids to about 950 amino acids, about 600 amino acids to about 900 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 1400 amino acids, about 650 amino acids to about 1350 amino acids, about 650 amino acids to about 1300 amino acids, about 650 amino acids to about 1250 amino acids, about 650 amino acids to about 1200 amino acids, about 650 amino acids to about 1150 amino acids, about 650 amino acids to about 1100 amino acids, about 650 amino acid to about 1050 amino acids, about 650 amino acids to about 1000 amino acids, about 650 amino acids to about 950 amino acids, about 650 amino acids to about 900 amino acids, about 650 amino acids to about 850 amino acids, about 650 amino acids to about 800 amino acids, about 650 amino acids to about 750 amino acids, about 650 amino acids to about 700 amino acids, about 700 amino acids to about 1400 amino acids, about 700 amino acids to about 1350 amino acids, about 700 amino acids to about 1300 amino acids, about 700 amino acids to about 1250 amino acids, about 700 amino acids to about 1200 amino acids, about 700 amino acids to about 1150 amino acids, about 700 amino acids to about 1100 amino acids, about 700 amino acid to about 1050 amino acids, about 700 amino acids to about 1000 amino acids, about 700 amino acids to about 950 amino acids, about 700 amino acids to about 900 amino acids, about 700 amino acids to about 850 amino acids, about 700 amino acids to about 800 amino acids, about 700 amino acids to about 750 amino acids, about 750 amino acids to about 1400 amino acids, about 750 amino acids to about 1350 amino acids, about 750 amino acids to about 1300 amino acids, about 750 amino acids to about 1250 amino acids, about 750 amino acids to about 1200 amino acids, about 750 amino acids to about 1150 amino acids, about 750 amino acids to about 1100 amino acids, about 750 amino acid to about 1050 amino acids, about 750 amino acids to about 1000 amino acids, about 750 amino acids to about 950 amino acids, about 750 amino acids to about 900 amino acids, about 750 amino acids to about 850 amino acids, about 750 amino acids to about 800 amino acids, about 800 amino acids to about 1400 amino acids, about 800 amino acids to about 1350 amino acids, about 800 amino acids to about 1300 amino acids, about 800 amino acids to about 1250 amino acids, about 800 amino acids to about 1200 amino acids, about 800 amino acids to about 1150 amino acids, about 800 amino acids to about 1100 amino acids, about 800 amino acid to about 1050 amino acids, about 800 amino acids to about 1000 amino acids, about 800 amino acids to about 950 amino acids, about 800 amino acids to about 900 amino acids, about 800 amino acids to about 850 amino acids, about 850 amino acids to about 1400 amino acids, about 850 amino acids to about 1350 amino acids, about 850 amino acids to about 1300 amino acids, about 850 amino acids to about 1250 amino acids, about 850 amino acids to about 1200 amino acids, about 850 amino acids to about 1150 amino acids, about 850 amino acids to about 1100 amino acids, about 850 amino acid to about 1050 amino acids, about 850 amino acids to about 1000 amino acids, about 850 amino acids to about 950 amino acids, about 850 amino acids to about 900 amino acids, about 900 amino acids to about 1400 amino acids, about 900 amino acids to about 1350 amino acids, about 900 amino acids to about 1300 amino acids, about 900 amino acids to about 1250 amino acids, about 900 amino acids to about 1200 amino acids, about 900 amino acids to about 1150 amino acids, about 900 amino acids to about 1100 amino acids, about 900 amino acid to about 1050 amino acids, about 900 amino acids to about 1000 amino acids, about 900 amino acids to about 950 amino acids, about 950 amino acids to about 1400 amino acids, about 950 amino acids to about 1350 amino acids, about 950 amino acids to about 1300 amino acids, about 950 amino acids to about 1250 amino acids, about 950 amino acids to about 1200 amino acids, about 950 amino acids to about 1150 amino acids, about 950 amino acids to about 1100 amino acids, about 950 amino acid to about 1050 amino acids, about 950 amino acids to about 1000 amino acids, about 1000 amino acids to about 1400 amino acids, about 1000 amino acids to about 1350 amino acids, about 1000 amino acids to about 1300 amino acids, about 1000 amino acids to about 1250 amino acids, about 1000 amino acids to about 1200 amino acids, about 1000 amino acids to about 1150 amino acids, about 1000 amino acids to about 1100 amino acids, about 1000 amino acid to about 1050 amino acids, about 1050 amino acids to about 1400 amino acids, about 1050 amino acids to about 1350 amino acids, about 1050 amino acids to about 1300 amino acids, about 1050 amino acids to about 1250 amino acids, about 1050 amino acids to about 1200 amino acids, about 1050 amino acids to about 1150 amino acids, about 1050 amino acids to about 1100 amino acids, about 1100 amino acids to about 1400 amino acids, about 1100 amino acids to about 1350 amino acids, about 1100 amino acids to about 1300 amino acids, about 1100 amino acids to about 1250 amino acids, about 1100 amino acids to about 1200 amino acids, about 1100 amino acids to about 1150 amino acids, about 1150 amino acids to about 1400 amino acids, about 1150 amino acids to about 1350 amino acids, about 1150 amino acids to about 1300 amino acids, about 1150 amino acids to about 1250 amino acids, about 1150 amino acids to about 1200 amino acids, about 1200 amino acids to about 1400 amino acids, about 1200 amino acids to about 1350 amino acids, about 1200 amino acids to about 1300 amino acids, about 1200 amino acids to about 1250 amino acids, about 1250 amino acids to about 1400 amino acids, about 1250 amino acids to about 1350 amino acids, about 1250 amino acids to about 1300 amino acids, about 1300 amino acids to about 1400 amino acids, about 1300 amino acids to about 1350 amino acids, or about 1350 amino acids to about 1400 amino acids.

For example, the β-TCP-binding polypeptide can include the amino acid sequence of LLADTTHHRPWTVIGESTHHRPWSIIGESSHHKPFTGLGDTTHHRPWGILAESTHHKPWTATGGSGEGGTGASTGGSAGTGGSGGTTSGEAGGSSGAG (SEQ ID NO: 44). In some instances the β-TCP-binding polypeptide includes an amino acid sequence that has at least 70% (e.g., at least 75%, at least 80%, at least 85%, at least 90%, at least 95%, at least 96%, at least 97%, at least 98%, at least 99%) sequence identity to SEQ ID NO: 44.

For example, the β-TCP-binding polypeptide may include: LLADTTHHRPWTGLGDTTHHRPWGLLADTTHHRPWTGLGDTTHHRPWGLLADTTHHRPWTTGGSGEGGTGASTGGSAGTGGSGGTTSGEAGGSSGAG (SEQ ID NO: 45). In some instances the β-TCP-binding polypeptide includes an amino acid sequence that has at least 70% (e.g., at least 75%, at least 80%, at least 85%, at least 90%, at least 95%, at least 96%, at least 97%, at least 98%, at least 99%) sequence identity to SEQ ID NO: 45.

A variety of different methods known in the art can be used to determine the K_{D} values of any of the β-TCP-binding polypeptides described herein for binding to β-TCP (e.g., any of the types of β-TCP described herein) (e.g., an electrophoretic mobility shift assay, a filter binding assay, surface plasmon resonance, and a biomolecular binding kinetics assay, etc.)

### Nucleic Acids/Vectors

Also provided herein are nucleic acids that encode any of the chimeric polypeptides or β-TCP-binding polypeptides described herein.

Also provided herein are vectors that include any of the nucleic acids provided herein. A “vector” according to the present disclosure is a polynucleotide capable of inducing the expression of a recombinant protein (e.g., any of the chimeric polypeptides or β-TCP-binding polypeptides described) in a host cell. A vector provided herein can be, e.g., in circular or linearized form. Non-limiting examples of vectors include plasmids, SV40 vectors, adenoviral viral vectors, and adeno-associated virus (AAV) vectors. Non-limiting examples of vectors include lentiviral vectors or retroviral vectors, e.g., gamma-retroviral vectors. See, e.g., Carlens et al., Exp. Hematol. 28(10:1137-1146, 2000; Park et al., Trends Biotechnol. 29(11):550-557, 2011; and Alonso-Camino et al., Mol. Ther. Nucleic Acids 2:e93, 2013. Non-limiting examples of retroviral vectors include those derived from Moloney murine leukemia virus, myeloproliferative sarcoma virus, murine embryonic stem cell virus, murine stem cell virus, spleen focus forming virus, or adeno-associated virus. Non-limiting examples of retroviral vectors are described in, e.g., U.S. Pat. Nos. 5,219,740 and 6,207,453; Miller et al., BioTechniques 7:980-990, 1989; Miller, Human Gene Therapy 1:5-14, 1990; Scarpa et al., Virology 180:849-852, 1991; Burns et al., Proc. Natl. Acad. Sci. U.S.A. 90:8033-8037, 1993; and Boris-Lawrie et al., Cur. Opin. Genet. Develop. 3:102-109, 1993. Exemplary lentiviral vectors are described in, e.g., Wang et al., J. Immunother. 35(9):689-701, 2003; Cooper et al., Blood 101:1637-1644, 2003; Verhoeyen et al., Methods Mol. Biol. 506:97-114, 2009; and Cavalieri et al., Blood 102(2):497-505, 2003.

Exemplary vectors, in which any of the nucleic acids provided herein can be inserted, are described in, e.g., Ausubel et al., Eds. “Current Protocols in Molecular Biology” Current Protocols, 1993; and Sambrook et al., Eds. “Molecular Cloning: A Laboratory Manual,” 2nd ed., Cold Spring Harbor Press, 1989.

In some embodiments, the vectors further include a promoter and/or enhancer operably linked to any of the nucleic acids described herein. Non-limiting examples of promoters include promoters from human cytomegalovirus (CMV), mouse phosphoglycerate kinase 1, polyoma adenovirus, thyroid stimulating hormone α, vimentin, simian virus 40 (SV40), tumor necrosis factor, β-globin, α-fetoprotein, γ-globin, β-interferon, γ-glutamyl transferase, human ubiquitin C (UBC), mouse mammary tumor virus (MMTV), Rous sarcoma virus, glyceraldehyde-3-phosphate dehydrogenase, β-actin, metallothionein II (MT II), amylase, human EF1α, cathepsin, MI muscarinic receptor, retroviral LTR (e.g. human T-cell leukemia virus HTLV), AAV ITR, interleukin-2, collagenase, platelet-derived growth factor, adenovirus E2, stromelysin, murine MX, rat insulin, glucose regulated protein 78, human immunodeficiency virus, glucose regulated protein 94, α-2-macroglobulin, MHC class I, HSP70, proliferin, immunoglobulin light chain, T-cell receptor, HLA DQα, HLA DQβ, interleukin-2 receptor, MHC class II, prealbumin (transthyretin), elastase I, albumin, c-fos, neural cell adhesion molecule (NCAM), H2B histone, rat growth hormone, human serum amyloid (SAA), muscle creatinine kinase, troponin I (TN I), and Gibbon Ape Leukemia Virus (GALV). In some embodiments, the promoter may be an inducible promoter or a constitutive promoter. Additional examples of promoters are known in the art.

In some examples, the vectors provided herein further include a poly(A) sequence, which is operably linked and positioned 3′ to the sequence encoding the chimeric polypeptide or β-TCP-binding polypeptide. Non-limiting examples of a poly(A) sequence include those derived from bovine growth hormone (Woychik et al., Proc. Natl. Acad. Sci. U.S.A. 81(13): 3944-3948, 1984, and U.S. Pat. No. 5,122, 458), mouse-β-globin, mouse-α-globin (Orkin et al., EMBO J. 4(2): 453-456, 1985), human collagen, polyoma virus (Batt et al., Mol. Cell Biol. 15(9):4783-4790, 1995), the Herpes simplex virus thymidine kinase gene (HSV TK), IgG heavy chain gene polyadenylation signal (U.S. Pat. Application Publication No. 2006/0040354), human growth hormone (hGH) (Szymanski et al., Mol. Therapy 15(7): 1340-1347, 2007), SV40 poly(A) site, e.g., SV40 late and early poly(A) site (Schek et al., Mol. Cell Biol. 12(12):5386-5393, 1992). In some embodiments, the poly(A) sequence includes a highly conserved upstream element (AATAAA). The this AATAAA sequence can, e.g., be substituted with other hexanucleotide sequences with homology to AATAAA which are capable of signaling polyadenylation, including, e.g., ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, and AATAAG. See, e.g., WO 06012414 A2). A poly(A) sequence can, e.g., be a synthetic polyadenylation site. See, e.g., Levitt el al, Genes Dev. 3(7): 1019-1025, 1989). In some examples, a poly(A) sequence can be the polyadenylation signal of soluble neuropilin-1: AAATAAAATACGAAATG. Additional examples of poly(A) sequences are known in the art. Additional examples and aspects of vectors are also known in the art.

### Methods of Making a Chimeric Polypeptide

Also provided herein are methods of making a chimeric polypeptide (e.g., any of the chimeric polypeptides described herein) or a β-TCP-binding polypeptide (e.g., any of the β-TCP-binding polypeptides described herein) that include: introducing into a cell a nucleic acid sequence encoding the chimeric polypeptide or the β-TCP-binding polypeptide to produce a recombinant cell; and culturing the recombinant cell under conditions sufficient for the expression of the chimeric polypeptide or β-TCP-binding polypeptide. In some embodiments, the introducing step includes introducing into a cell an expression vector including a nucleic acid sequence encoding the chimeric polypeptide or the β-TCP-binding polypeptide to produce a recombinant cell. In some embodiments, the expression vector includes chaperones (e.g., GroES, GroEL) and glutathione to aid with in vitro folding.

A chimeric polypeptide or β-TCP-binding polypeptide described herein can be produced by any cell, e.g., a eukaryotic cell or a prokaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. In some embodiments, the eukaryotic cell is a mammalian cell (e.g., a Chinese Hamster Ovary (CHO) cell). As used herein, the term “prokaryotic cell” refers to a cell that does not have a distinct, membrane-bound nucleus. In some embodiments, the prokaryotic cell is a bacterial cell. In some embodiments, the bacterial cell is a chemically competent E.coli K12 cell (e.g., Shuffle® T7; New England BioLabs) or a BL21(DE3) pLysS chemically competent E.coli cell.

Methods of culturing cells are well known in the art. Cells can be maintained in vitro under conditions that favor proliferation, differentiation, and growth. Briefly, cells can be cultured by contacting a cell (e.g., any cell) with a cell culture medium that includes the necessary growth factors and supplements to support cell viability and growth.

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalection, hydrodynamic delivery, magnetofection, viral transduction (e.g., adenoviral and lentiviral transduction), and nanoparticle transfection.

Provided herein are methods that further include isolation of the chimeric polypeptide or the β-TCP-binding polypeptide from a cell (e.g., a eukaryotic cell) using techniques well-known in the art (e.g., ammonium sulfate precipitation, polyethylene glycol precipitation, cobalt column, heparin column, ion-exchange chromatography (anion or cation), chromatography based on hydrophobic interaction, metal-affinity chromatography, ligand-affinity chromatography, and size exclusion chromatography).

In some embodiments, a hydrophobicity/hydrophilicity plot is constructed to determine variant β-TCP binding sequences that can be included in any of the chimeric polypeptides described herein.

### β-TCP

Sintering of tricalcium phosphate, Ca₃(PO₄)₂, causes its structure to convert to β-TCP. β-TCP is an osteoconductive material that supports bone mineralization by easily dissolving at low pH and serves as a rigid substrate for cell attachment (see, e.g., Muschler et al., J. Bone Joint Surgery 86:1541-1558, 2004; Fleming Jr. et al., “Intraoperative Harvest and Concentration of Human Bone Marrow Osteoprogenitors for Enhancement of Spinal Fusion,” in Orthopedic Tissue Engineering: Basic Science and Practice, 2004).

β-TCP as described herein can be in a variety of different forms. Examples of such forms include a granular form, a porous form, a powder, a putty (e.g., a moldable putty), a paste, a scaffold, and/or a coating on a solid surface (e.g., a coating on a medical device). In addition, the β-TCP can be used in a variety of different shapes (e.g., a cross, a ladder, a sphere, an ellipsoid, a square, a triangular pyramid, a rod, a cone, a torus, or a wedge, or any combination thereof) and sizes (e.g., largest average diameter of about 1 mm, about 2 mm, about 3 mm, about 4 mm, about 5 mm, about 6 mm, about 7 mm, about 8 mm, about 9 mm, about 1 cm, about 2 cm, about 3 cm, about 4 cm, about 5 cm, about 6 cm, about 7 cm, about 8 cm, about 9 cm, or about 10 cm). In some embodiments, β-TCP can be porous (e.g., about 20% to about 80% porous, about 20% to about 75% porous, about 20% to about 70% porous, about 20% to about 65% porous, about 20% to about 60% porous, about 20% to about 55% porous, about 20% to about 50% porous, about 20% to about 45% porous, about 20% to about 40% porous, about 20% to about 35% porous, about 20% to about 30% porous, about 20% to about 25% porous, about 25% to about 80% porous, about 25% to about 75% porous, about 25% to about 70% porous, about 25% to about 65% porous, about 25% to about 60% porous, about 25% to about 55% porous, about 25% to about 50% porous, about 25% to about 45% porous, about 25% to about 40% porous, about 25% to about 35% porous, about 25% to about 30% porous, about 30% to about 80% porous, about 30% to about 75% porous, about 30% to about 70% porous, about 30% to about 65% porous, about 30% to about 60% porous, about 30% to about 55% porous, about 30% to about 50% porous, about 30% to about 45% porous, about 30% to about 40% porous, about 30% to about 35% porous, about 35% to about 80% porous, about 35% to about 75% porous, about 35% to about 70% porous, about 35% to about 65% porous, about 35% to about 60% porous, about 35% to about 55% porous, about 35% to about 50% porous, about 35% to about 45% porous, about 35% to about 40% porous, about 40% to about 80% porous, about 40% to about 75% porous, about 40% to about 70% porous, about 40% to about 65% porous, about 40% to about 60% porous, about 40% to about 55% porous, about 40% to about 50% porous, about 40% to about 45% porous, about 45% to about 80% porous, about 45% to about 75% porous, about 45% to about 70% porous, about 45% to about 65% porous, about 45% to about 60% porous, about 45% to about 55% porous, about 45% to about 50% porous, about 50% to about 80% porous, about 50% to about 75% porous, about 50% to about 70% porous, about 50% to about 65% porous, about 50% to about 60% porous, about 50% to about 55% porous, about 55% to about 80% porous, about 55% to about 75% porous, about 55% to about 70% porous, about 55% to about 65% porous, about 55% to about 60% porous, about 60% to about 80% porous, about 60% to about 75% porous, about 60% to about 70% porous, about 60% to about 65% porous, about 65% to about 80% porous, about 65% to about 75% porous, about 65% to about 70% porous, about 70% to about 80% porous, about 70% to about 75% porous, or about 75% to about 80% porous). For example, β-TCP can have an average pore size of about 1 nm to about 50 mm, about 1 nm to about 45 mm, about 1 nm to about 40 mm, about 1 nm to about 35 mm, about 1 nm to about 30 mm, about 1 nm to about 25 mm, about 1 nm to about 20 mm, about 1 nm to about 15 mm, about 1 nm to about 10 mm, about 1 nm to about 5 mm, about 1 nm to about 1 mm, about 1 nm to about 900 Tm, about 1 nm to about 800 Tm, about 1 nm to about 700 Tm, about 1 nm to about 600 Tm, about 1 nm to about 500 Tm, about 1 nm to about 400 Tm, about 1 nm to about 300 Tm, about 1 nm to about 200 Tm, about 1 nm to about 100 Tm, about 1 nm to about 50 Tm, about 1 nm to about 10 Tm, about 1 nm to about 5 Tm, about 1 nm to about 1 Tm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 300 nm, about 1 nm to about 200 nm, about 1 nm to about 100 nm, about 1 nm to about 50 nm, about 1 nm to about 25 nm, about 1 nm to about 10 nm, about 1 nm to about 5 nm, about 5 nm to about 50 mm, about 5 nm to about 45 mm, about 5 nm to about 40 mm, about 5 nm to about 35 mm, about 5 nm to about 30 mm, about 5 nm to about 25 mm, about 5 nm to about 20 mm, about 5 nm to about 15 mm, about 5 nm to about 10 mm, about 5 nm to about 5 mm, about 5 nm to about 1 mm, about 5 nm to about 900 Tm, about 5 nm to about 800 Tm, about 5 nm to about 700 Tm, about 5 nm to about 600 Tm, about 5 nm to about 500 Tm, about 5 nm to about 400 Tm, about 5 nm to about 300 Tm, about 5 nm to about 200 Tm, about 5 nm to about 100 Tm, about 5 nm to about 50 Tm, about 5 nm to about 10 Tm, about 5 nm to about 5 Tm, about 5 nm to about 1 Tm, about 5 nm to about 900 nm, about 5 nm to about 800 nm, about 5 nm to about 700 nm, about 5 nm to about 600 nm, about 5 nm to about 500 nm, about 5 nm to about 400 nm, about 5 nm to about 300 nm, about 5 nm to about 200 nm, about 5 nm to about 100 nm, about 5 nm to about 50 nm, about 5 nm to about 25 nm, about 5 nm to about 10 nm, about 10 nm to about 50 mm, about 10 nm to about 45 mm, about 10 nm to about 40 mm, about 10 nm to about 35 mm, about 10 nm to about 30 mm, about 10 nm to about 25 mm, about 10 nm to about 20 mm, about 10 nm to about 15 mm, about 10 nm to about 10 mm, about 10 nm to about 5 mm, about 10 nm to about 1 mm, about 10 nm to about 900 Tm, about 10 nm to about 800 Tm, about 10 nm to about 700 Tm, about 10 nm to about 600 Tm, about 10 nm to about 500 Tm, about 10 nm to about 400 Tm, about 10 nm to about 300 Tm, about 10 nm to about 200 Tm, about 10 nm to about 100 Tm, about 10 nm to about 50 Tm, about 10 nm to about 10 Tm, about 10 nm to about 5 Tm, about 10 nm to about 1 Tm, about 10 nm to about 900 nm, about 10 nm to about 800 nm, about 10 nm to about 700 nm, about 10 nm to about 600 nm, about 10 nm to about 500 nm, about 10 nm to about 400 nm, about 10 nm to about 300 nm, about 10 nm to about 200 nm, about 10 nm to about 100 nm, about 10 nm to about 50 nm, about 10 nm to about 25 nm, about 25 nm to about 50 mm, about 25 nm to about 45 mm, about 25 nm to about 40 mm, about 25 nm to about 35 mm, about 25 nm to about 30 mm, about 25 nm to about 25 mm, about 25 nm to about 20 mm, about 25 nm to about 15 mm, about 25 nm to about 10 mm, about 25 nm to about 5 mm, about 25 nm to about 1 mm, about 25 nm to about 900 Tm, about 25 nm to about 800 Tm, about 25 nm to about 700 Tm, about 25 nm to about 600 Tm, about 25 nm to about 500 Tm, about 25 nm to about 400 Tm, about 25 nm to about 300 Tm, about 25 nm to about 200 Tm, about 25 nm to about 100 Tm, about 25 nm to about 50 Tm, about 25 nm to about 10 Tm, about 25 nm to about 5 Tm, about 25 nm to about 1 Tm, about 25 nm to about 900 nm, about 25 nm to about 800 nm, about 25 nm to about 700 nm, about 25 nm to about 600 nm, about 25 nm to about 500 nm, about 25 nm to about 400 nm, about 25 nm to about 300 nm, about 25 nm to about 200 nm, about 25 nm to about 100 nm, about 25 nm to about 50 nm, about 50 nm to about 50 mm, about 50 nm to about 45 mm, about 50 nm to about 40 mm, about 50 nm to about 35 mm, about 50 nm to about 30 mm, about 50 nm to about 25 mm, about 50 nm to about 20 mm, about 50 nm to about 15 mm, about 50 nm to about 10 mm, about 50 nm to about 5 mm, about 50 nm to about 1 mm, about 50 nm to about 900 Tm, about 50 nm to about 800 Tm, about 50 nm to about 700 Tm, about 50 nm to about 600 Tm, about 50 nm to about 500 Tm, about 50 nm to about 400 Tm, about 50 nm to about 300 Tm, about 50 nm to about 200 Tm, about 50 nm to about 100 Tm, about 50 nm to about 50 Tm, about 50 nm to about 10 Tm, about 50 nm to about 5 Tm, about 50 nm to about 1 Tm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, about 50 nm to about 100 nm, about 100 nm to about 50 mm, about 100 nm to about 45 mm, about 100 nm to about 40 mm, about 100 nm to about 35 mm, about 100 nm to about 30 mm, about 100 nm to about 25 mm, about 100 nm to about 20 mm, about 100 nm to about 15 mm, about 100 nm to about 10 mm, about 100 nm to about 5 mm, about 100 nm to about 1 mm, about 100 nm to about 900 Tm, about 100 nm to about 800 Tm, about 100 nm to about 700 Tm, about 100 nm to about 600 Tm, about 100 nm to about 500 Tm, about 100 nm to about 400 Tm, about 100 nm to about 300 Tm, about 100 nm to about 200 Tm, about 100 nm to about 100 Tm, about 100 nm to about 50 Tm, about 100 nm to about 10 Tm, about 100 nm to about 5 Tm, about 100 nm to about 1 Tm, about 100 nm to about 900 nm, about 100 nm to about 800 nm, about 100 nm to about 700 nm, about 100 nm to about 600 nm, about 100 nm to about 500 nm, about 100 nm to about 400 nm, about 100 nm to about 300 nm, about 100 nm to about 200 nm, about 200 nm to about 50 mm, about 200 nm to about 45 mm, about 200 nm to about 40 mm, about 200 nm to about 35 mm, about 200 nm to about 30 mm, about 200 nm to about 25 mm, about 200 nm to about 20 mm, about 200 nm to about 15 mm, about 200 nm to about 10 mm, about 200 nm to about 5 mm, about 200 nm to about 1 mm, about 200 nm to about 900 Tm, about 200 nm to about 800 Tm, about 200 nm to about 700 Tm, about 200 nm to about 600 Tm, about 200 nm to about 500 Tm, about 200 nm to about 400 Tm, about 200 nm to about 300 Tm, about 200 nm to about 200 Tm, about 200 nm to about 100 Tm, about 200 nm to about 50 Tm, about 200 nm to about 10 Tm, about 200 nm to about 5 Tm, about 200 nm to about 1 Tm, about 200 nm to about 900 nm, about 200 nm to about 800 nm, about 200 nm to about 700 nm, about 200 nm to about 600 nm, about 200 nm to about 500 nm, about 200 nm to about 400 nm, about 200 nm to about 300 nm, about 300 nm to about 50 mm, about 300 nm to about 45 mm, about 300 nm to about 40 mm, about 300 nm to about 35 mm, about 300 nm to about 30 mm, about 300 nm to about 25 mm, about 300 nm to about 20 mm, about 300 nm to about 15 mm, about 300 nm to about 10 mm, about 300 nm to about 5 mm, about 300 nm to about 1 mm, about 300 nm to about 900 Tm, about 300 nm to about 800 Tm, about 300 nm to about 700 Tm, about 300 nm to about 600 Tm, about 300 nm to about 500 Tm, about 300 nm to about 400 Tm, about 300 nm to about 300 Tm, about 300 nm to about 200 Tm, about 300 nm to about 100 Tm, about 300 nm to about 50 Tm, about 300 nm to about 10 Tm, about 300 nm to about 5 Tm, about 300 nm to about 1 Tm, about 300 nm to about 900 nm, about 300 nm to about 800 nm, about 300 nm to about 700 nm, about 300 nm to about 600 nm, about 300 nm to about 500 nm, about 300 nm to about 400 nm, about 400 nm to about 50 mm, about 400 nm to about 45 mm, about 400 nm to about 40 mm, about 400 nm to about 35 mm, about 400 nm to about 30 mm, about 400 nm to about 25 mm, about 400 nm to about 20 mm, about 400 nm to about 15 mm, about 400 nm to about 10 mm, about 400 nm to about 5 mm, about 400 nm to about 1 mm, about 400 nm to about 900 Tm, about 400 nm to about 800 Tm, about 400 nm to about 700 Tm, about 400 nm to about 600 Tm, about 400 nm to about 500 Tm, about 400 nm to about 400 Tm, about 400 nm to about 300 Tm, about 400 nm to about 200 Tm, about 400 nm to about 100 Tm, about 400 nm to about 50 Tm, about 400 nm to about 10 Tm, about 400 nm to about 5 Tm, about 400 nm to about 1 Tm, about 400 nm to about 900 nm, about 400 nm to about 800 nm, about 400 nm to about 700 nm, about 400 nm to about 600 nm, about 400 nm to about 500 nm, about 500 nm to about 50 mm, about 500 nm to about 45 mm, about 500 nm to about 40 mm, about 500 nm to about 35 mm, about 500 nm to about 30 mm, about 500 nm to about 25 mm, about 500 nm to about 20 mm, about 500 nm to about 15 mm, about 500 nm to about 10 mm, about 500 nm to about 5 mm, about 500 nm to about 1 mm, about 500 nm to about 900 Tm, about 500 nm to about 800 Tm, about 500 nm to about 700 Tm, about 500 nm to about 600 Tm, about 500 nm to about 500 Tm, about 500 nm to about 400 Tm, about 500 nm to about 300 Tm, about 500 nm to about 200 Tm, about 500 nm to about 100 Tm, about 500 nm to about 50 Tm, about 500 nm to about 10 Tm, about 500 nm to about 5 Tm, about 500 nm to about 1 Tm, about 500 nm to about 900 nm, about 500 nm to about 800 nm, about 500 nm to about 700 nm, about 500 nm to about 600 nm, about 600 nm to about 50 mm, about 600 nm to about 45 mm, about 600 nm to about 40 mm, about 600 nm to about 35 mm, about 600 nm to about 30 mm, about 600 nm to about 25 mm, about 600 nm to about 20 mm, about 600 nm to about 15 mm, about 600 nm to about 10 mm, about 600 nm to about 5 mm, about 600 nm to about 1 mm, about 600 nm to about 900 Tm, about 600 nm to about 800 Tm, about 600 nm to about 700 Tm, about 600 nm to about 600 Tm, about 600 nm to about 500 Tm, about 600 nm to about 400 Tm, about 600 nm to about 300 Tm, about 600 nm to about 200 Tm, about 600 nm to about 100 Tm, about 600 nm to about 50 Tm, about 600 nm to about 10 Tm, about 600 nm to about 5 Tm, about 600 nm to about 1 Tm, about 600 nm to about 900 nm, about 600 nm to about 800 nm, about 600 nm to about 700 nm, about 700 nm to about 50 mm, about 700 nm to about 45 mm, about 700 nm to about 40 mm, about 700 nm to about 35 mm, about 700 nm to about 30 mm, about 700 nm to about 25 mm, about 700 nm to about 20 mm, about 700 nm to about 15 mm, about 700 nm to about 10 mm, about 700 nm to about 5 mm, about 700 nm to about 1 mm, about 700 nm to about 900 Tm, about 700 nm to about 800 Tm, about 700 nm to about 700 Tm, about 700 nm to about 600 Tm, about 700 nm to about 500 Tm, about 700 nm to about 400 Tm, about 700 nm to about 300 Tm, about 700 nm to about 200 Tm, about 700 nm to about 100 Tm, about 700 nm to about 50 Tm, about 700 nm to about 10 Tm, about 700 nm to about 5 Tm, about 700 nm to about 1 Tm, about 700 nm to about 900 nm, about 700 nm to about 800 nm, about 800 nm to about 50 mm, about 800 nm to about 45 mm, about 800 nm to about 40 mm, about 800 nm to about 35 mm, about 800 nm to about 30 mm, about 800 nm to about 25 mm, about 800 nm to about 20 mm, about 800 nm to about 15 mm, about 800 nm to about 10 mm, about 800 nm to about 5 mm, about 800 nm to about 1 mm, about 800 nm to about 900 Tm, about 800 nm to about 800 Tm, about 800 nm to about 700 Tm, about 800 nm to about 600 Tm, about 800 nm to about 500 Tm, about 800 nm to about 400 Tm, about 800 nm to about 300 Tm, about 800 nm to about 200 Tm, about 800 nm to about 100 Tm, about 800 nm to about 50 Tm, about 800 nm to about 10 Tm, about 800 nm to about 5 Tm, about 800 nm to about 1 Tm, about 800 nm to about 900 nm, about 900 nm to about 50 mm, about 900 nm to about 45 mm, about 900 nm to about 40 mm, about 900 nm to about 35 mm, about 900 nm to about 30 mm, about 900 nm to about 25 mm, about 900 nm to about 20 mm, about 900 nm to about 15 mm, about 900 nm to about 10 mm, about 900 nm to about 5 mm, about 900 nm to about 1 mm, about 900 nm to about 900 Tm, about 900 nm to about 800 Tm, about 900 nm to about 700 Tm, about 900 nm to about 600 Tm, about 900 nm to about 500 Tm, about 900 nm to about 400 Tm, about 900 nm to about 300 Tm, about 900 nm to about 200 Tm, about 900 nm to about 100 Tm, about 900 nm to about 50 Tm, about 900 nm to about 10 Tm, about 900 nm to about 5 Tm, about 900 nm to about 1 Tm, about 1 Tm to about 50 mm, about 1 Tm to about 45 mm, about 1 Tm to about 40 mm, about 1 Tm to about 35 mm, about 1 Tm to about 30 mm, about 1 Tm to about 25 mm, about 1 Tm to about 20 mm, about 1 Tm to about 15 mm, about 1 Tm to about 10 mm, about 1 Tm to about 5 mm, about 1 Tm to about 1 mm, about 1 Tm to about 900 Tm, about 1 Tm to about 800 Tm, about 1 Tm to about 700 Tm, about 1 Tm to about 600 Tm, about 1 Tm to about 500 Tm, about 1 Tm to about 400 Tm, about 1 Tm to about 300 Tm, about 1 Tm to about 200 Tm, about 1 Tm to about 100 Tm, about 1 Tm to about 50 Tm, about 1 Tm to about 10 Tm, about 1 Tm to about 5 Tm, about 5 Tm to about 50 mm, about 5 Tm to about 45 mm, about 5 Tm to about 40 mm, about 5 Tm to about 35 mm, about 5 Tm to about 30 mm, about 5 Tm to about 25 mm, about 5 Tm to about 20 mm, about 5 Tm to about 15 mm, about 5 Tm to about 10 mm, about 5 Tm to about 5 mm, about 5 Tm to about 1 mm, about 5 Tm to about 900 Tm, about 5 Tm to about 800 Tm, about 5 Tm to about 700 Tm, about 5 Tm to about 600 Tm, about 5 Tm to about 500 Tm, about 5 Tm to about 400 Tm, about 5 Tm to about 300 Tm, about 5 Tm to about 200 Tm, about 5 Tm to about 100 Tm, about 5 Tm to about 50 Tm, about 5 Tm to about 10 Tm, about 10 Tm to about 50 mm, about 10 Tm to about 45 mm, about 10 Tm to about 40 mm, about 10 Tm to about 35 mm, about 10 Tm to about 30 mm, about 10 Tm to about 25 mm, about 10 Tm to about 20 mm, about 10 Tm to about 15 mm, about 10 Tm to about 10 mm, about 10 Tm to about 5 mm, about 10 Tm to about 1 mm, about 10 Tm to about 900 Tm, about 10 Tm to about 800 Tm, about 10 Tm to about 700 Tm, about 10 Tm to about 600 Tm, about 10 Tm to about 500 Tm, about 10 Tm to about 400 Tm, about 10 Tm to about 300 Tm, about 10 Tm to about 200 Tm, about 10 Tm to about 100 Tm, about 10 Tm to about 50 Tm, about 50 Tm to about 50 mm, about 50 Tm to about 45 mm, about 50 Tm to about 40 mm, about 50 Tm to about 35 mm, about 50 Tm to about 30 mm, about 50 Tm to about 25 mm, about 50 Tm to about 20 mm, about 50 Tm to about 15 mm, about 50 Tm to about 10 mm, about 50 Tm to about 5 mm, about 50 Tm to about 1 mm, about 50 Tm to about 900 Tm, about 50 Tm to about 800 Tm, about 50 Tm to about 700 Tm, about 50 Tm to about 600 Tm, about 50 Tm to about 500 Tm, about 50 Tm to about 400 Tm, about 50 Tm to about 300 Tm, about 50 Tm to about 200 Tm, about 50 Tm to about 100 Tm, about 100 Tm to about 50 mm, about 100 Tm to about 45 mm, about 100 Tm to about 40 mm, about 100 Tm to about 35 mm, about 100 Tm to about 30 mm, about 100 Tm to about 25 mm, about 100 Tm to about 20 mm, about 100 Tm to about 15 mm, about 100 Tm to about 10 mm, about 100 Tm to about 5 mm, about 100 Tm to about 1 mm, about 100 Tm to about 900 Tm, about 100 Tm to about 800 Tm, about 100 Tm to about 700 Tm, about 100 Tm to about 600 Tm, about 100 Tm to about 500 Tm, about 100 Tm to about 400 Tm, about 100 Tm to about 300 Tm, about 100 Tm to about 200 Tm, about 200 Tm to about 50 mm, about 200 Tm to about 45 mm, about 200 Tm to about 40 mm, about 200 Tm to about 35 mm, about 200 Tm to about 30 mm, about 200 Tm to about 25 mm, about 200 Tm to about 20 mm, about 200 Tm to about 15 mm, about 200 Tm to about 10 mm, about 200 Tm to about 5 mm, about 200 Tm to about 1 mm, about 200 Tm to about 900 Tm, about 200 Tm to about 800 Tm, about 200 Tm to about 700 Tm, about 200 Tm to about 600 Tm, about 200 Tm to about 500 Tm, about 200 Tm to about 400 Tm, about 200 Tm to about 300 Tm, about 300 Tm to about 50 mm, about 300 Tm to about 45 mm, about 300 Tm to about 40 mm, about 300 Tm to about 35 mm, about 300 Tm to about 30 mm, about 300 Tm to about 25 mm, about 300 Tm to about 20 mm, about 300 Tm to about 15 mm, about 300 Tm to about 10 mm, about 300 Tm to about 5 mm, about 300 Tm to about 1 mm, about 300 Tm to about 900 Tm, about 300 Tm to about 800 Tm, about 300 Tm to about 700 Tm, about 300 Tm to about 600 Tm, about 300 Tm to about 500 Tm, about 300 Tm to about 400 Tm, about 400 Tm to about 50 mm, about 400 Tm to about 45 mm, about 400 Tm to about 40 mm, about 400 Tm to about 35 mm, about 400 Tm to about 30 mm, about 400 Tm to about 25 mm, about 400 Tm to about 20 mm, about 400 Tm to about 15 mm, about 400 Tm to about 10 mm, about 400 Tm to about 5 mm, about 400 Tm to about 1 mm, about 400 Tm to about 900 Tm, about 400 Tm to about 800 Tm, about 400 Tm to about 700 Tm, about 400 Tm to about 600 Tm, about 400 Tm to about 500 Tm, about 500 Tm to about 50 mm, about 500 Tm to about 45 mm, about 500 Tm to about 40 mm, about 500 Tm to about 35 mm, about 500 Tm to about 30 mm, about 500 Tm to about 25 mm, about 500 Tm to about 20 mm, about 500 Tm to about 15 mm, about 500 Tm to about 10 mm, about 500 Tm to about 5 mm, about 500 Tm to about 1 mm, about 500 Tm to about 900 Tm, about 500 Tm to about 800 Tm, about 500 Tm to about 700 Tm, about 500 Tm to about 600 Tm, about 600 Tm to about 50 mm, about 600 Tm to about 45 mm, about 600 Tm to about 40 mm, about 600 Tm to about 35 mm, about 600 Tm to about 30 mm, about 600 Tm to about 25 mm, about 600 Tm to about 20 mm, about 600 Tm to about 15 mm, about 600 Tm to about 10 mm, about 600 Tm to about 5 mm, about 600 Tm to about 1 mm, about 600 Tm to about 900 Tm, about 600 Tm to about 800 Tm, about 600 Tm to about 700 Tm, about 700 Tm to about 50 mm, about 700 Tm to about 45 mm, about 700 Tm to about 40 mm, about 700 Tm to about 35 mm, about 700 Tm to about 30 mm, about 700 Tm to about 25 mm, about 700 Tm to about 20 mm, about 700 Tm to about 15 mm, about 700 Tm to about 10 mm, about 700 Tm to about 5 mm, about 700 Tm to about 1 mm, about 700 Tm to about 900 Tm, about 700 Tm to about 800 Tm, about 800 Tm to about 50 mm, about 800 Tm to about 45 mm, about 800 Tm to about 40 mm, about 800 Tm to about 35 mm, about 800 Tm to about 30 mm, about 800 Tm to about 25 mm, about 800 Tm to about 20 mm, about 800 Tm to about 15 mm, about 800 Tm to about 10 mm, about 800 Tm to about 5 mm, about 800 Tm to about 1 mm, about 800 Tm to about 900 Tm, about 900 Tm to about 50 mm, about 900 Tm to about 45 mm, about 900 Tm to about 40 mm, about 900 Tm to about 35 mm, about 900 Tm to about 30 mm, about 900 Tm to about 25 mm, about 900 Tm to about 20 mm, about 900 Tm to about 15 mm, about 900 Tm to about 10 mm, about 900 Tm to about 5 mm, about 900 Tm to about 1 mm, about 1 mm to about 50 mm, about 1 mm to about 45 mm, about 1 mm to about 40 mm, about 1 mm to about 35 mm, about 1 mm to about 30 mm, about 1 mm to about 25 mm, about 1 mm to about 20 mm, about 1 mm to about 15 mm, about 1 mm to about 10 mm, about 1 mm to about 5 mm, about 5 mm to about 50 mm, about 5 mm to about 45 mm, about 5 mm to about 40 mm, about 5 mm to about 35 mm, about 5 mm to about 30 mm, about 5 mm to about 25 mm, about 5 mm to about 20 mm, about 5 mm to about 15 mm, about 5 mm to about 10 mm, about 10 mm to about 50 mm, about 10 mm to about 45 mm, about 10 mm to about 40 mm, about 10 mm to about 35 mm, about 10 mm to about 30 mm, about 10 mm to about 25 mm, about 10 mm to about 20 mm, about 10 mm to about 15 mm, about 15 mm to about 50 mm, about 15 mm to about 45 mm, about 15 mm to about 40 mm, about 15 mm to about 35 mm, about 15 mm to about 30 mm, about 15 mm to about 25 mm, about 15 mm to about 20 mm, about 20 mm to about 50 mm, about 20 mm to about 45 mm, about 20 mm to about 40 mm, about 20 mm to about 35 mm, about 20 mm to about 30 mm, about 20 mm to about 25 mm, about 25 mm to about 50 mm, about 25 mm to about 45 mm, about 25 mm to about 40 mm, about 25 mm to about 35 mm, about 25 mm to about 30 mm, about 30 mm to about 50 mm, about 30 mm to about 45 mm, about 30 mm to about 40 mm, about 30 mm to about 35 mm, about 35 mm to about 50 mm, about 35 mm to about 45 mm, about 35 mm to about 40 mm, about 40 mm to about 50 mm, about 40 mm to about 45 mm, or about 45 mm to about 50 mm.

In some embodiments, the β-TCP is about 50% to about 70% porous, with the pores having a mean pore diameter of about 50 Tm to about 70 Tm (e.g., about 55 Tm to about 65 Tm, or about 60 Tm).

The invention is further described in the following examples, which do not limit the scope of the invention described in the claims.

### EXAMPLES

### Example 1. A Preclinical Study of tBMP-2 in a β-TCP Carrier in a Rat Critical Size Femoral Defect Model

The aim of this study was to examine the safety and effect of modified recombinant human bone morphogenetic protein (rhBMP) known as tBMP in a beta-tricalcium phosphate (β-TCP) putty matrix in the replacement and repair of bone in a rat critical size defect model. The tBMP-2 variant exhibits very tight binding to calcium phosphate ceramics and thus allows for targeted delivery of tBMP-2 to implant sites. This was achieved by surgically creating the defect using the RatFix internal fixator system, then filling that defect with tBMP + β -TCP putty, or β -TCP putty alone as a control. The positive control was a dose-adjusted commercially available (Medtronic) preparation for human use of rhBMP with an absorbable collagen sponge (ACS). The animals were monitored clinically and had bi-weekly radiographs until their scheduled end-points at four or eight weeks post-administration of treatment. Ex vivo histology was conducted on organs and tissues, and the treated leg was subjected to µCT, histomorphometry, and/or mechanical strength testing.

This report covers animal arrival, assignment to groups, surgical creation of defect and administration of test, reference or control item. In addition, this report covers the post-operative period up to and including Week 8, and end-point in vivo and ex vivo measurements. Included are the clinical summaries, radiographs, end-point clinical pathology and organ histopathology, histomorphometry of treated legs (odd-numbered animals), and mechanical strength testing of treated legs (even-numbered animals).

On arrival at the Test Facility, all animals underwent a Veterinary health check and were weighed. Based on the weights, the animals were assigned to groups A) tBMP + β -TCP putty; B) rhBMP +ACS; or C) β -TCP putty alone. The animals then had at least five days to acclimatize to their new surroundings and diet.

On the day of surgery, the animals were anaesthetized, the defect was created and the bone supported with the internal fixator following the method supplied by the manufacturer with some refinements to the surgical procedure. The pre-prepared test, reference or control item was then inserted into the defect. The surgical site was closed and the animal went immediately for X-ray. Following completion of the baseline radiograph, post-operative pain relief was administered and the animal was returned to its cage for recovery. Post-operative analgesia and antibiotic therapy were continued for at least two days, and longer when required.

A number of the original animals were removed from the study and replaced prior to surgery as they were getting too large for the fixator system specifications. The second, replacement group also had a thorough Veterinary health check and were weighed prior to being assigned to groups. The allocation of animals to either four or eight-week end-point groups was adjusted to include animals from both weight ranges at both end-points.

A total of 61 animals were successfully administered assigned treatment and recovered from surgery. Of these, one animal was euthanized five days post-operatively due to poor recovery and inappetence. The animal was replaced. Two animals which were found to have dislodged bone plates during their 2-week post-operative radiographs were humanely killed and not replaced. Two animals did not successfully recover from surgery, one died immediately post-surgery, and the other had an unstable bone plate which could not be fixed, and so was humanely killed. Both of these animals were replaced immediately.

The animals were monitored at least once daily, and their clinical signs scored. Any animal with unusual findings, or considered to be not eating and gaining weight normally was monitored more frequently, and veterinary advice was sought. When unusual findings were noted, this resulted in possible treatment with additional antibiotic or analgesic therapy, fluid supplementation), and in four cases euthanasia (three broken bones and one with continued weight loss).

All relevant animals received bone-labelling dyes at 10 days (calcein green) and 3 days (xylenol orange) prior to their scheduled end-point date. All animals had end-point blood samples collected. There were no clinically significant differences found between any of the groups for any biochemical or haematological parameter at either end-point time. Three haematology samples were clotted and could not be analysed.

In all specimens examined, there was evidence of extensive mineralization akin to mature trabecular bone in samples from animals in the tBMP + β -TCP putty group (Group A). This mineralization appears to be lamella (mature) bone rather than the woven bone that occurs in early callus formation (presence of osteocytes). Group A animals also had osteoclasts present (suggesting active remodelling), and red blood cells and adipocytes, indicating angiogenesis and infiltration of cells to most regions of the callus. Whilst there is mineralized bone in the specimens from the rhBMP +ACS group animals, there are extensive regions of un-mineralized fibrous and cartilaginous tissue within the callus region. The callus development in these samples appears to be at an earlier stage than that observed in the Group A animals. The Group C (β -TCP putty) samples show no evidence of mineralization, bone formation or bone remodeling even at eight weeks post-application of treatment.

The mechanical testing analyses showed the tBMP+ β -TCP group animals (Group A) had significantly stronger bones at the four and eight week end-point times than either of the other treatment groups.

In conclusion, the bone specimens form the tBMP+ β -TCP putty group clearly demonstrated vastly improved bone healing at 4 and 8 weeks post-surgery when compared to samples from animals in the rhBMP+ACS or the β -TCP putty alone treatment groups. The aim of this study was to evaluate the performance of a modified variant of recombinant human BMP-2 (rhBMP-2) called tBMP-2. The tBMP-2 variant exhibits very tight binding to calcium phosphate ceramics and thus allows for targeted delivery of tBMP-2 to implant sites. The ability to tether BMP-2 in this manner will allow for longer persistence times, lower doses, and, it is expected, superior outcomes.

### Animals

The rat is a validated animal model for assessing the effect of treatments on critical-sized defects in the femur (4-6). Sixty male rats (plus spares) were required: ten animals in each group per time point. One animal was replaced after successful administration of the test item at day 5 post-surgery as he was losing weight and not eating. Animals that were euthanized or humanely killed after successful administration of the test item were necropsied and had tissue collected for histopathology. The number of animals used in this study was considered sufficient for evaluation of results.

The animals were housed in conventional conditions (targeted temperature 22 ± 3° C., 12/12-hour light/dark cycle) in standard open top cages that satisfy the size requirements specified in the Animal Welfare Regulations (Animal Welfare Act 1985, v 15.10.2015. Attorney General’s Department, Gouverment of South Australia). Animals were housed individually for the entirety of the experiment. The animals had access to standard laboratory rat chow (Specialty Feeds, Glen Forest, Australia) 25-30 g/day. Immediately post-operatively, and when rats were not thriving, the food was soaked in water for easier palatability. Chlorinated tap water was provided to the animals ad libitum. Food and water were not withheld at any time.

Each animal was uniquely identified by a subcutaneously implanted microchip which was scanned using a barcode reader. For the purpose of the study, each animal was also given a number from 1-70 (to include replacement and spared required). As the initial batch of animals gained weight too quickly, some animals were replaced prior to surgery. The replacement animals were given the number of the animal they replaced and the suffix (a) was added. Numbers went up to 68a with a couple of gaps.

Animals were assigned to groups by a weight-ordered distribution. The heavier animals were used first. The animals gained weight rapidly, even on the restricted food allowance. Two weeks into the surgeries, it was deemed necessary to remove some animals from the study (n=16) as these animals were already too heavy (>425 g). These animals were replaced with animals in the 250-300 g range. The replacement animals were also assigned to treatment groups on a weight-ordered distribution. As the first surgical group had originally been assigned to the 8-week end-point group, animals were reassigned to either 4-or 8- week end-points to ensure the lighter animals were evenly distributed amongst the treatment and end-point times.

### Analgesia/Antibiotic Therapy

All interventional procedures were performed under isoflurane in O₂ anaesthesia. Induction and maintenance of surgical depth anesthesia was at 2-4% isoflurane (Baxter International, Sydney, Australia) in a flow of 1-2 L/minute O₂. Post-operatively, after closure of the wound, a topical application of local anesthetic in the form of Marcaine (Bupivacaine, 0.5%, AstraZeneca, Frewville, SA, Australia) up to 2.5 mg/Kg was applied to the area around the wound. The animals received 0.1 mg/kg buprenorphine (Temgesic, Reckitt-Benckiser, Melrose Park, Australia) subcutaneously post-operatively after X-ray. They also received 0.1 mg/kg subcutaneously twice daily for two days post-operatively. Additionally, they were given a non-steroidal anti-inflammatory treatment in the form of Carprieve (Carprofen, 50 mg/mL, Norbrook Laboratories, Tullamarine, Australia) at a dose of 5 mg/kg subcutaneously post-operatively and once daily for at least two days post-surgery.

All animals received cephalosporin (Cefazolin, Hospira Inc, Lake Forest IL, USA) 20 mg/kg subcutaneously intra-operatively and twice daily for two days post-operatively. Any animals that needed re-suturing of their wounds received antibiotic treatment for up to fourteen days post-repair. Antibiotic therapy was ceased if suspected to be causing diarrhea and/or weight loss.

### Microchip Implantation

The microchip was inserted into the scruff of the neck (after clipping hair) using a microchip implanter. The microchip was inserted until completely covered by skin. The wounds were closed with one or two wound clips if required. The area was swabbed with betadine. This was performed under isoflurane in O₂ anaesthesia at the same time as the defect surgery and test- or reference-item placement.

### Critical-Size Defect Creation

The defects were created in the right femur of all rats as described for the RatFix RISystem. Briefly, all anaesthetized rats were placed on a warming pad in lateral recumbency with the right leg facing upwards. The surgical site was shaved and aseptically prepared with iodine or chlorhexidine scrub and solution. A skin incision between the greater trochanter and the knee joint was made and the superficial fascia incised. The intermuscular plane between the vastus lateralis and the biceps femoris was separated and the periosteum of the femur incised. The PEEK plate was fitter into the jug and secured with suture material. The jig-plate assembly was fixed to the craniolateral surface of the femur by pulling the sutures through under the femur, allowing the assembly to be tightened to the femur.

After predilling the holes in the PEEL plate using the supplied drill bit, the plate was attached to the femur by six bicortical titanium screws. Standardised 6-mm defects were created (marked on the plate) using the Gigli wire saw guided by the sawing device of the jig. After defect sawing, the jig and bone piece were removed. The fresh defect was flushed with sterile saline and dried with gauze in preparation of test item or reference item, or vehicle administration into the defect size. In the event that the plate itself was damaged with the cutting wire, reducing stability, that animal would be replaced. No replacements due to plate damage were required.

### Baseline Radiographs

Baseline radiographs were taken immediately after creation of the defect and administration of the test or reference item to show position of treatment articles (where visible), and placement of the plate, whilst the animal was still under anaesthesia.

### Radiographic Assessments

X-rays were taken on the anaesthetized animals (isoflurane) immediately after surgery, and at 2, 4 (end-point for half of the animals), 6 and 8 weeks (end-point for the remaining animals) post-operatively. The radiographs were taken in lateromedial and craiocaudal projections for assessment of bone healing and to exclude implant loosening or failure. All in-life radiographs were taken using a Villa Visitor Mobile X-ray Unit (Villa Sistemi Medicali, Buccinasco, Italy), using a dental image capture device (Soredex, Digora Optime, Tuusula, Finland). The images were transferred to the PIRL picture archiving and communication system (PACS, Carestream Vue Motion, Rochester, USA) by the radiographer for storage and access.

### Bone Labelling

At 10 days prior to their prescribed end-point, all animals received an intraperitoneal injection of calcein green (25 mg/kg in saline). At 3 days prior to their prescribed end-point all animals received an injection of xylenol orange (10 mg/mL, 25 mg/kg, intraperitoneal) to double-label the bone for histomorphometric analyses.

### Blood Collection/ Haematology

All blood collections were performed on anaesthetized animals at their end-point radiograph. Blood was collected via cardiac puncture.

Blood samples were evaluated for the parameters specified in Tables 1 and 2. For Table 1, samples of approximately 2 mL were collected into tubes containing K₂EDTA anticoagulant for haemotological analyses. Analyses were performed on an Abbott Cell Dyn. 3700 (Abbott Laboratories, North Ryde, Australia). For Table 2, samples of approximately 5 mL were collected into tubes containing a clot activator for biochemical analysis. Analyses were performed on a Siemens Advia 1800 (Siemens Healthcare Diagnostics Inc., Flanders, NJ, USA).

TABLE 1

Haematology Parameters to be reported

| Haemoglobin (Hb) | White Cell Count (WCC) |
|---|---|
| Erythrocyte count (RBC) | Mean Corpuscular Volume (MCV) |
| Packed Cell Volume (PCV)/ Haematocrit (HCT) | Mean Corpuscular Haemoglobin (MCH)* |
| Mean Corpuscular Haemoglobin Concentration (MCHC)* | Red Cell Distribution Width (RDW) |
| Platelets (Plt) | |

White Blood Cell Differential: Neutrophils, Lymphocytes, Monocytes, Eosinophils, Basophils
*Calculated values

TABLE 2

Serum Chemistry Parameters to be reported

| | | |
|---|---|---|
| Electrolytes: | Anion Gap* (AG) | Albumin (Alb) |
| Sodium (Sod) | Glucose (Gluc) | Globulin* (Glob) |
| Potassium (Pot) | Urea | Protein (Tot Prot) |
| Chloride (Chl) | Creatinine (Creat) | Total bilirubin (Tot Bili) |
| Bicarbonate (Bicarb) | Cholesterol (Chol) | Lactate Dehydrogenase (LD) |
| Lipid Studies: | Urate (Uric Acid, UA) | Alkaline phosphatase (ALP) |
| Triglycerides (Trig) | Phosphate (Phos) | Total Calcium (Tot. Ca) |
| High Density Lipoproteins (HDL) | Gamma glutamyltransferase (GGT) | Alanine aminotransferase (ALT) |
| Low Density Lipoproteins (LDL) | Asparatate aminotransferase (AST) | |
| Chol/HDL * | *Calculated values | |

### Mechanical Strength Testing

This was performed on half of the animals at each time point. All tissue was placed in 0.9% NaCl-soaked gauzed in 50-mL sterile urine pots to avoid freezer burn and stored frozen at - 20° C. until testing. Tissue was prepared immediately and frozen within one hour of collection to avoid autolysis. Immediately before testing, tissues were thawed, and the internal fixator device carefully cut in the middle section. Tissue was test at room temperature (approximately 23° C.). The ultimate breaking strength was measured by using a load frame (model 5542, Instron, Canton, MA) and a 3-point bend fixture (model 2810-400, Instron) at a crosshead speed of 10 mm/min. The load cell for this testing (Instron 2530-416) had a maximum capacity of 500 N. The data (force in kg and extension in mm) was collected and analysed with a vendor-provided commercial mathematical software package (Bluehill2, Instron).

### Histological Analyses

Specimen were fixed in 10% formalin for 7 days prior to processing. Formalin-fixed bones were cut using a slow-speed saw (Buehler Ltd, IL, USA) along the sagittal plane using a diamond-tipped cutting blade before being submerged in 70% ethanol. Subsequently, bones were processed for resin embedding via several dehydration steps. Briefly, bones were submerged in 2 × 90% ethanol and 1 × 100% ethanol steps over 48 hours. Bones were then transferred into a solution containing methymethacrylate (MMA) and 10% v/v polyethylene glycol (PEG) and stored at room temperature for 10-14 days. Resin embedding then occurs by preparing a solution of MMA, 10% PEG, and 0.4% peroxydicarbonate (Perkadox) and incubation at 37° C. for 24 hours to allow the resin to harden. The exposed cut surface was placed facing down in the tube. The resin-set bones were then removed from their tubes and fixed to stubs for sectioning. For each bone, 5-µm thick sections were cut using a tungsten-carbide blade (Leica RM2255, Wetzlar, GER). Sections were placed on to gel-coated slides and dipped 2 times in a spreading solution (70% ethanol/30% 2-ethoxyethanol) heated to 70° C. To ensure adherence to the slide, sections were flattended, covered by strips of polyethylene and clamped together separated by blotting paper, before being placed into a 37° C. oven. Prior to commencing staining procedures, sections were placed in acetone for 15 minutes, unless otherwise stated. All sections were digitally scanned at 100x magnification (3D Histech scanner, TMA-MASTER01).

### Von Koss + Haemaotoxylin and Eosin (H&E) Staining

Acetone treated sections were rinsed in demineralised water for 2 minutes. Sections were then placed in a 1% silver nitrate solution and placed in front of a UV lamp for 60 minutes. Washed slides were then treated with 2.5% sodium thiosulphate solution for 5 minutes. Washed slides were then counterstained with H&E. To stain for H&E, sections were placed in haematoxylin for 8 minutes to stain cell nuclei before being rinsed in demineralised water for 2 minutes and dipped in acid alcohol (%), typically, 4-5 minutes. After rinsing in demineralised water, sections were dipped in lithium carbonate 4-5 times and placed in eosin for 4 minutes. Once removed, excess eosin was removed with a squeeze bottle of absolute alcohol, sections were hydrated, placed in xylene and mounted in xylene-based mounting medium.

### Tartrate-resistant Acid Phosphatase (TRAP) Stain for Analysis of Osteoclasts

Acetone treated sections underwent a 60-minute incubation in Tris-HCL buffer (pH 9.4) at 37° C. for 60 minutes in an acid phosphatase (AcP) stain prepared by adding 0.0355 g of tartaric acid dissolved in 35 mL of sodium acetate (pH 5.2) to 100 µL basic fucshin in a 100 µL solution containing 0.4 mg of sodium nitrite. This solution was then added to a solution containing 0.04 g Napthol ASBI phosphate (Sigma-Aldrich, Missouri, USA) in 2 mL dimethylformamide. Subsequently, two washed were performed before the sections were counterstained in haematoxylin for 8 minutes, then rinsed in demineralised water, then dipped in acid alcohol 4 times, rinsed again, dipped in lithium carbonate 4 times and then dehydrated, placed in xylene and mounted in DPX. Quantification of osteoclast number per bone perimeter mm was performed by identifying cells (stained pink-red) on the surface of bone (stained blue-purple) and calculated by the OsteoMeasure histomorphometry system.

### Double Fluorochrome Labelling of Bone Sections For Measures of Bone Formation

Acetone sections were immediately placed in xylene and mounted in DPX. Slides were viewed under a fluorescent microscope (Olympus BX53; Olympus, Tokyo, JP).

### Histomorphometry

Those animals which did not have mechanical testing of femurs will undergo histological analysis of the defect and treatments. Following µCT of the femurs, they were opened at the wound site, and the internal fixator device carefully removed. They were placed in 70% ethanol. Femurs were placed in PMMA, polymethyl methacrylate (10% polyethylene glycol [PEG] in methylmethacrylate) for 10-14 days, then polymerized in PMMA containing 10% Perkadox 16 (di[4-tert-butylcyclohexyl] peroxydicarbonate) at 37° C. for 24 hours. Sections were stained with H&E, and tartrate resistant acid phosphatase (TRAP) for analysis.

### Humane Killing

Animals were humanely killed on day 28 ± 2 days (n=30) or day 56 ± 3 (n=30) immediately following their end-point radiograph and blood collection, and whilst still under anaesthesia, with an intracariac injection of a lethal dose of sodium pentobarbitone of 60 mg/mL formulation (Lethabarb®) at 200 mg/kg. Death was confirmed by lack of respiration and palpable heartbeat, loss of corneal reflex and loss of colour of mucous membranes.

### Necropsy

All animals were subjected to a comprehensive necropsy. A comprehensive necropsy is defined as examination of the external surface of the body, all orfices, and the cranial, thoracic, and abdominal cavities, and their contents. If abnormalities were found in tissues or organs other than those listed below, they were also collected for histology.

### Organ and Tissue Collection

Whole organs, sections of the issues listed in Table 3 below were dissected free and fixed in 10% neutral buffered formalin. This was done for twelve animals from each groups (six animals from each group at each time-point). The animals were selected such that at least one from each group was selected at each end-point (staggered as surgeries were staggered).

TABLE 3

Organ and Tissue Samples Collected From Each Animal

| | | |
|---|---|---|
| Adrenal gland | Lesions | Skin (containing implant[s]) |
| Brain | Liver | Spinal cord (cervical, thoracic, and lumbar) |
| Cecum | Lymph node (mesenteric) | Spleen |
| Duodenum | Pancreas | Sternum (+ bone marrow) |
| Eye | Pituitary Gland | Stomach |
| Heart | Rectum | Thymus |
| Ileum | Salivary gland (mandibular) | Tongue |
| Jejunum | Sciatic nerve | Epididymis |
| Kidney | Skeletal muscle (thigh) | |
| Femur with bone marrow (articular surface of the distal end) | Lung (with mainstem bronchi) | Prostate/seminal vesicles, bladder, testes |

### Treated Limb Collection and Storage

Immediately following confirmation of death, the treated leg was removed and placed in either 10% neutral buffered formalin (n=5 animals/group/time point), or wrapped in saline-soaked gauze and placed at -20° C. for at least 48 hours (n=10 animals/group/time point).

### µCt

All legs had µCT measurements (Bruker Skyscan 1076, Brussels, Belgium) of the defect performed to quantify newly mineralized bone volume. This was done on all animals at each end-point time. The bones were either formalin-fixed (105 neutral buffered formalin) or frozen at -20° C. for 3-5 days prior to testing. The plate was adjusted to the longitudinal axis of the device. Scanning parameters and intensity were recorded and were in the vicinity of a source voltage of 70 kV with an intensity of 114 µA. The end-point µCT was manually and subjectively scored as described by Chhabra et al. (2005) using the grading system described in Table 4.

TABLE 4

Radiographic Grading Scale of Fracture Callus Formation

| Grade | Amount of Callus Formation | Grade | Amount of Callus Formation |
|---|---|---|---|
| 0 | No callus | 3 | Bridging periosteal callus |
| 1 | Little-to-moderate callus | 4 | Mature callus with interfragmentary bridging |
| 2 | Profuse callus tissue | 5 | Callus resorption after solid union |

### Study Design

The study was performed as outlined in Table 5. There were three groups with 20 animals/group. All animals underwent surgery to create a critical-size defect (6-mm) in the mid diaphysis of the right femur (RatFix RISystem), and had inserted into the defect test item, reference item or vehicle. The animals were monitored at least once daily and were weighed at least twice weekly. They underwent radiographic evaluation immediately post-operatively, and at 2, 4, 6, and 8 weeks post-surgery. Ten animals from each group (n=30 animals) were scheduled to have end-point data (µCT, mechanical testing, histomorphometry, clinical pathology) collected 4 weeks post-operatively and the remaining animals (n=30 animals) were scheduled for end-point data collection 8-weeks post operatively. Of these, six from each group (chosen such that there was a representative from each group from the majority of surgical days) at each end-point had tissues and organs collected for histology.

TABLE 5

Study design

| Group | Treatment | Nomenclature | End-Point (n) | |
|---|---|---|---|---|
| | | | Week 4 | Week 8 |
| A | tBMP-2 + β-TCP putty | tBMP-2 + β-TCP putty | 10 | 10 |
| B | rhBMP-2 + absorbable collagen sponge (ACS) | rhBMP2 + ACS | 10 | 10 |
| C | β-TCP putty | β-TCP putty | 10 | 10 |

Group A animals received tBMP-2+β-TCP (0.615-0.620 mg tBMP-2 in β-TCP putty to fill defect); Group B animals received InFUSE™ Bone Graft of rhBMP-2 +ACS (2 µg rhBMP-2 on ACS to fill defect); Group C animals received β-TCP to fill the defect. All test and control items were mixed on the day of surgery (tBMP-coated β-TCP to putty or rhBMP to ACS). Following administration of the treatment article, the wounds were closed in two muscle layers with subcutis and intracutaneous vicryl sutures. The wounds were closed exteriorly with intradermal sutures to prevent chewing by the rats. The wounds were washed with liberal amounts chlorhexidine solution. A small amount of tea tree oil was applied to the area surrounding the actual wound to prevent the animals from worrying the suture side.

### Route of Administration

The anticipated route of human administration is by surgical implantation into a bone defect. Therefore, that route was used in this study. The bone defects were surgically created, the area flushed with saline, then the test or control items were placed in the defect immediately.

### Preparation of Test Item (tBMP-2 + β-TCP Putty)

For each rat, 195 µL of tBMP-2 (stock 3.53 mg/mL) were added to a pre-weighed aliquot of 47 mg β -TCP granules and mixed gently for 2-3 hours. All liquid was removed, and retained for analysis. The pellet was washed with 1 mL of sterile phosphate-buffered saline (PBS pH 7.4) and mixed gently to wash away any excess unbound protein. As much as possible of the liquid was removed with a pipette, and sterile pre-weighed putty was added to the protein-coated β -TCP. β-TCP and putty were mixed 1:1. The entire tBMP-2 + β-TCP putty formulation was placed in the surgically-created defect. Assuming approximately 90% binding, the final dose of tBMP which was administered to the rat in the defect was 615-620 µg. Sterility was maintained at all steps. β-TCP binding to tBMP-2 was done no more than 48 hours prior to surgery. If β-TCP was bound/washed the day before surgery, it was stored at 4° C. β-TCP and putty were not mixed more than one hour prior to implantation, as it will dehydrate with time and become less malleable.

### Reference Item

Recombinant human bone morphogenetic protein + absorbable collagen sponge (InFUSE™ Bone Graft; size XX small; Medtronic Sofamor Danek, Inc) was used as per packet insert with modifications as described below. BMP-2 vial contents were reconstituted with the provided sterile water to give 1.5 mg/mL rhBMP-2. This concentration is intended for human use. To bring this concentration to the range typically used in rats, it was diluted 1 in 60 with PBS (25 µg/mL). A defect volume of approximately 75.5 mm³ was assumed, therefore the ACS was trimmed to form a 6-mm × 4-mm block for insertion into the defect. Diluted rhBMP solution (80 µL) were added to the ACS in a dropwise fashion at least 15 minutes prior to insertion into the defect. This resulted in 2 µg tBMP-2 in the defect. Sterility was maintained.

### β-TCP Putty

β-TCP granules (46-48 mg aliquots), carboxymethyl cellulose putty (48-50 mg aliquots). Under sterile conditions, the β-TCP granules were washed with 1 mL of sterile PBS and mixed gently. As much as possible of the liquid was removed using a pipette. Pre-weighed sterile carboxymethyl cellulose (approximately 2 mg more than the β-TCP putty formulation was inserted into the defect.

### Dose Formulation

The rhBMP-2 + absorbable collagen sponge and the tBMP-2 + β-TCP putty were prepared as described above. The final dose each rat received of the BMP-2 formulations was 2 µg rhBMP-2 for the reference item and approximately 615-620 µg tBMP of the test item.

### Animal Assignments and Body Weights

On arrival, the initial animals were numbered 1-63, of which 60 were assigned to the study. The animals were fed ad lib on arrival, but this was reduced to 35-30 g when it was found at their third weighing that they were gaining weight too quickly. The initial surgeries were delayed by one week, and as the animals weights continued to increase, the decision was made to remove the heaviest sixteen animals from the study and get replacement animals. The replacement animals were assigned numbers.

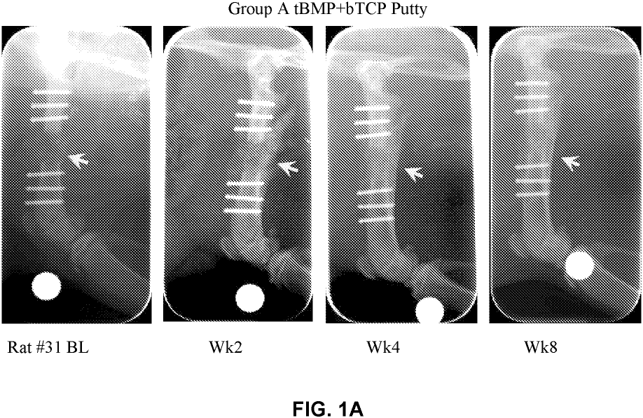

All animals were weighed on arrival and assigned to one of three groups on a weight-ordered distribution. All allocations to group and end-point times were done to ensure there were no significant differences in weight prior to the surgical intervention between any of the groups in terms of treatment to be administered, or time to end-point (either 4- or 8-weeks post-surgery). There were no significant differences in weights at assignment to study between any of the groups for either the initial group of animals or the replacement group, or at any time for the duration of the study (FIG. 1A, FIG. 1B and FIG. 1C).

### Example 2. Radiographic, Mechanical, Histomorphometric and Histological Analyses of 4 and 8 Week Post-Fracture Healing in Three Experimental Rat Fracture Groups (A, B andC)

To assess the extent of mineralized bone material within the callus region of fractures in three experimental rat groups (A, B and C), radiographic scoring of callus formation, mechanical testing of the callus strength, high resolution micro computed tomography (µCT), and quantitative histological analyses of bone formation and resorption were conducted.

### Radiographic Assessment

Sagittal radiographic images were generated using a Faxitron X-ray with fixed exposure settings. Radiographic scoring specifically on callus formation healing was based on Chhabra et al. (2005) J Orthop Trauma 19(9): 629-634, and modified from Marino et al. (1979) Clin Orthop Relat Res (145): 239-244 and Makley et al. (1967) J Bone Joint Surg Am 49(5): 903-914 (Table 4). Scoring was performed in one session on de-identified, randomised radiographs. Both the posterior and anterior aspect of the cortex at the site of fracture was scored. The average score for each specimen was recorded.

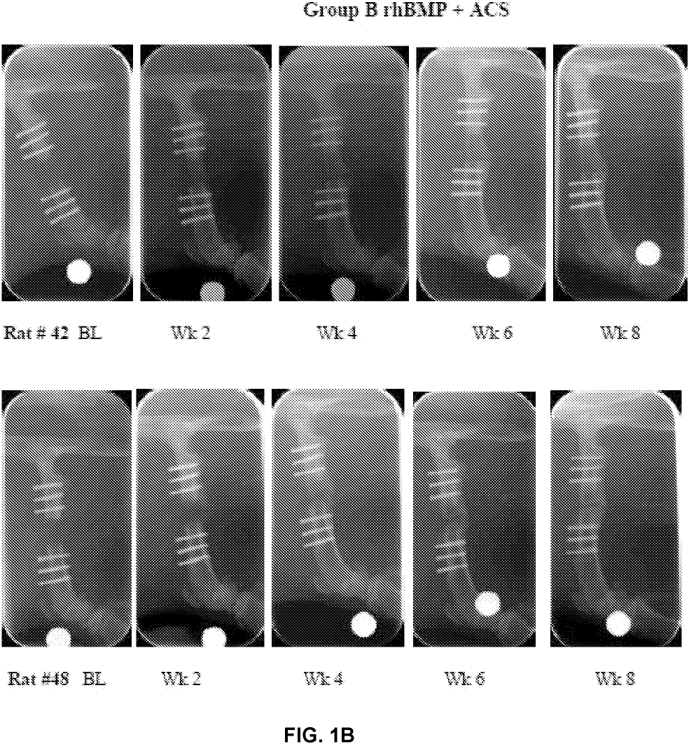

Baseline radiographs were taken on all animals as soon as possible after surgery whilst still under anaesthetic, at two weeks, four weeks (end-point for half of the animals), six weeks and eight weeks (end-point for remaining animals) post-surgery. They were taken in lateromedial and craniocaudal views. FIG. 2 shows representative radiographs of animals from all groups at 0, 2, 4, 6, and 8 weeks post-surgery. The defect was only visible in Group B animals that received the rhBMP + ACS. The putty (with or without protein) in animals from Groups A and C appear to be outside the confines of the defect (more obvious in mediolateral view). This was the case in all animals, even when the surgeon reported that the putty appeared to be completely contained within the defect, and touching the muscle tissue had been completely avoided on insertion of the test or control items.

Ex vivo radiographs, as with the in vivo radiographs showed that the putty and/or bone growth was not confined to the defect area in Groups A and C, as it was in Group B images (FIG. 2).

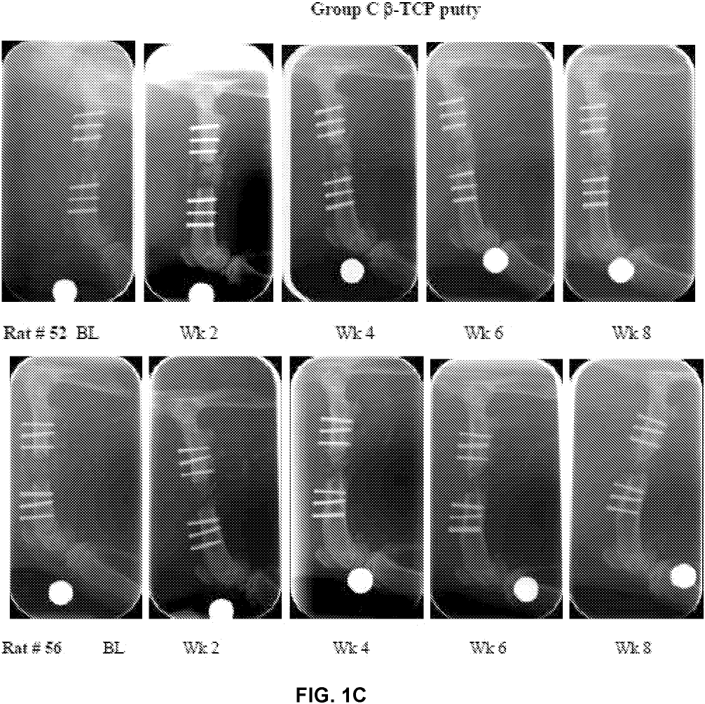

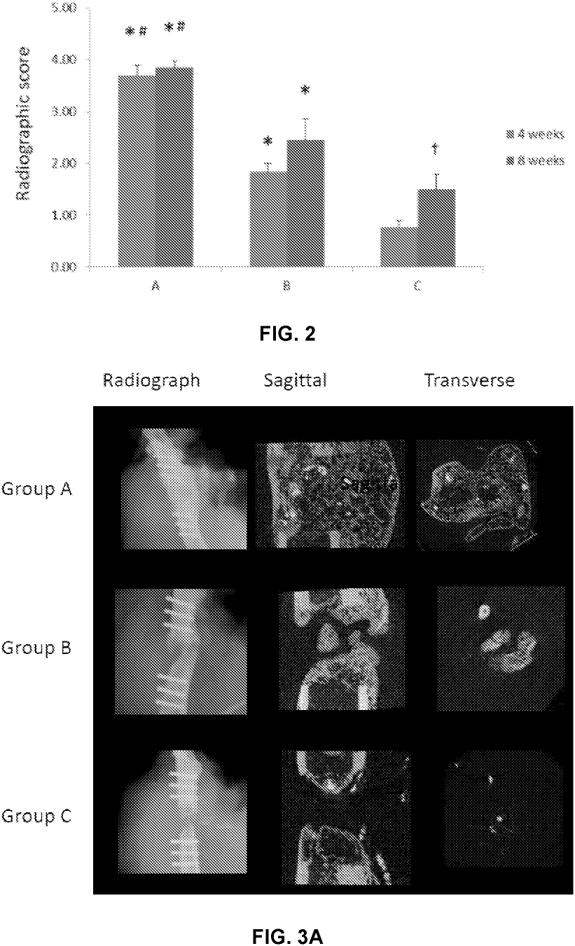

At 4-weeks post-surgery, Group A routinely demonstrated bridging of the periosteal callus and frequently demonstrated mature callus with interfragmentary bridging (FIG. 3A). This as in clear constrast to Groups B and C where only callus tissue and little or no callus scores were observed respectively. At 8-weeks post-surgery, while there appears to be no significant radiographic change in the scoring of the mature callus, Group A remained advanced in the healing stage when compared to Group B and C, both of which only marginally improved their scores in the intervening 4 weeks (FIG. 3B). It is worth noting that in Group A, frequent larger boney callus volumes were observed intact but distal to the fracture site suggesting that the treatment in this group was not contained to the fracture region. Representative images from each group at 4- and 8-weeks post-surgery are located in FIGS. 3A and 3B. A schematic of the varied callus formations is represented in FIG. 4.

### Micro-Computed Tomography

To quantify the total callus and bone mineral volumes, each specimen was scanned by high resolution µCT (Skyscan, Model 1174, Bruker microCT). Specimens were wrapped in PBS-soaked gauze and secured within a humidified tube prior to scanning. All scans were performed at 6.4 µm voxel resolution and X-ray tube potential of 50 kV and 800 µA. Images were acquired over 180 degrees with intervals of 0.8 degrees and frames were averaged from 2 images taken at each step. For each scan, the images were then reconstructed into a z-stack of images to represent the transverse plane of the femur (N-Recon software, Bruker microCT). All reconstructions used a modified Feldkamp cone-beam algorithm with a smoothing setting of 1, a ring artefact reduction level set to 12, a beam hardening level of 20% and a post-alignment balue of no greater than ± 1.0. Reconstructions of the callus region excluded the adjacent screws which secured the rat-fix plate, in order to remove the interfering signal of the titanium screws from subsequent analyses. Uniform realignment of datasets were performed using Dataviewer v.1.5.1 (Bruker, BEL). Reconstructed z-stack images for each specimen were then analysed using CTan software (Bruker microCT). Analyses of bone volume in the fracture region were performed by three manually defined volumes of interest (VOI’s). Prior to 3D analyses, all volumes of interest binarised using an adaptive thresholding technique using a specimen-specific hydroxyapatite standard control. 3D analyses were performed to establish total volume and bone volume fractions in each volume of interest.

During necropsy, all treated legs were removed, and placed in either 10% neutral-buffered formalin and kept at room temperature, or gauze-soaked saline and frozen at -20° C., for µCT imaging and mechanical strength testing. All legs were also X-rayed (Faxitron X-ray Imager) and the images graded as per Table 3 above. FIG. 4 shows a graphical representation of bridging of periosteal callus. Moderate callus was observed in Group B samples, whereas Group C samples showed little or no callus.

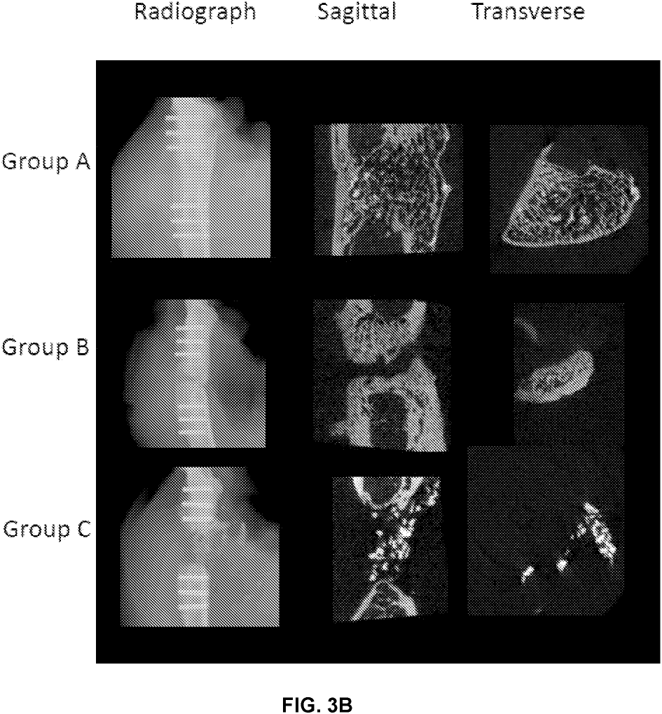

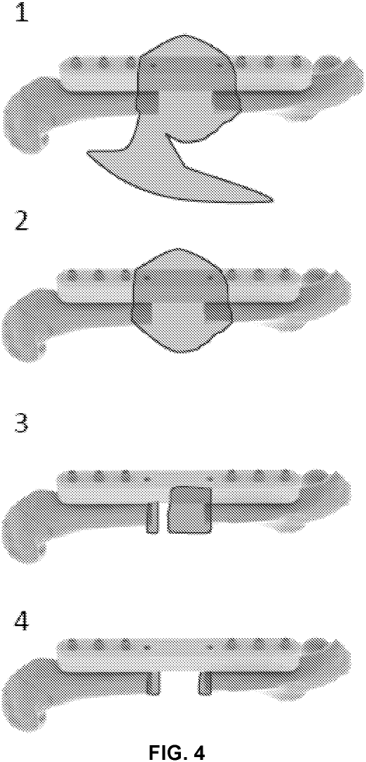

Analyses of the µCT images for bone volume in the fracture region showed that the total callus volume and the bone mineral volume were significantly higher in Group A (tBMP + β-TCP putty-treated) animals than in either the β-TCP putty (Group C) or the rhBMP + ACS-treated animals (p<0.01 in both cases). There were no significant differences between the Group B and Group C animals for either total callus volume or bone mineral volume. These are shown in FIG. 5A and FIG. 5B. When restricting the region to the interpolated shaft region, in both Group A and Group B, the bone mineral volume as a percentage of the tissue volume (BV/TV) was approximately 2-fold greater than Group C (P<0.05). The BV/TV for Group A was trending to be increased by 15% when compared to Group B (P=0.059) (FIG. 6A). When restricting the region to the interpolated cortical bone region only, in Group A, the cortical bone mineral volume, in BV/TV terms, was 4-fold greater than levels in Group B (P<0.05) but not significantly greater than in Group C (FIG. 6B).

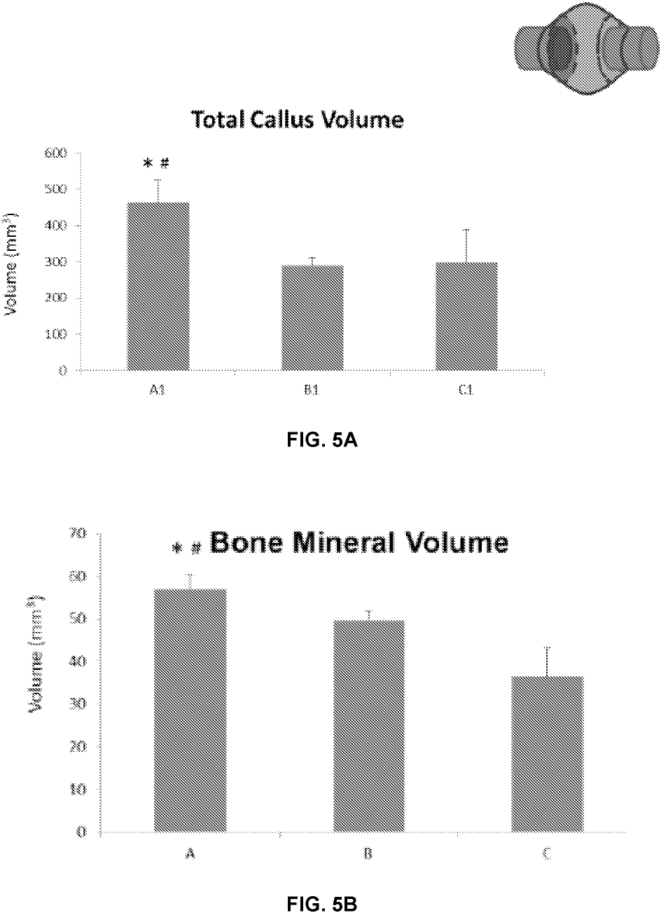

At 8-weeks post-surgery, TCB was markedly increased in Group A when compared to Groups B and C (FIG. 7A and FIG. 7B). However, BMV in Group A is not significantly increased when compared to Group B. These data suggested that while a larger bony callus existed in Group A, the density of bone mineral was more comparable to Group B.

### Mechanical Strength Testing of Treated Femur

The mechanical strength testing of femoral callus was performed using 3 point bending method (5940 and BlueHill 3 software, v3.25, Instron, MA, USA). Each specimen was stored in PBS-soaked gauze at -80° C. until ready for testing. Prior to testing, specimens were gradually thawed to 4° C. over three days to maintain tissue integrity. On the day of testing, samples were scanned by micro-CT in a humidified chamber and kept cool prior to allowing specimens to reach room temperature for mechanical testing. For each specimen, the rat-fix plate was cut immediately prior to testing using a slow speed rotary diamond tipped saw lubricated with cooled phosphate buffered saline. The mid-point of the rat-fix plate was cut to remove the support of the plate in the fracture stabilization. This cut was done so as to cause minimal disturbance to the surrounding callus. After cutting the plate, each bone was then loaded into the 3-point jig with the rat-fix plate in contact with the low anvils with a 10 mm span such that the upper anvil was positioned directly above the cut-point on the plate. The downward motion of the upper anvil tests the resistance, and thus the strength, of the callus. As the bone deforms during testing, the plate separates at the mid-point cut, minimizing the contribution of the plate to the measure of Ultimate Load to Failure (ULF). T Compression testing was performed by gradually increasing the force applied, at a rate of 0.01 mm per second. ULF was determined by the peak force immediately prior to the failure of callus as determined by the force-displacement curve.

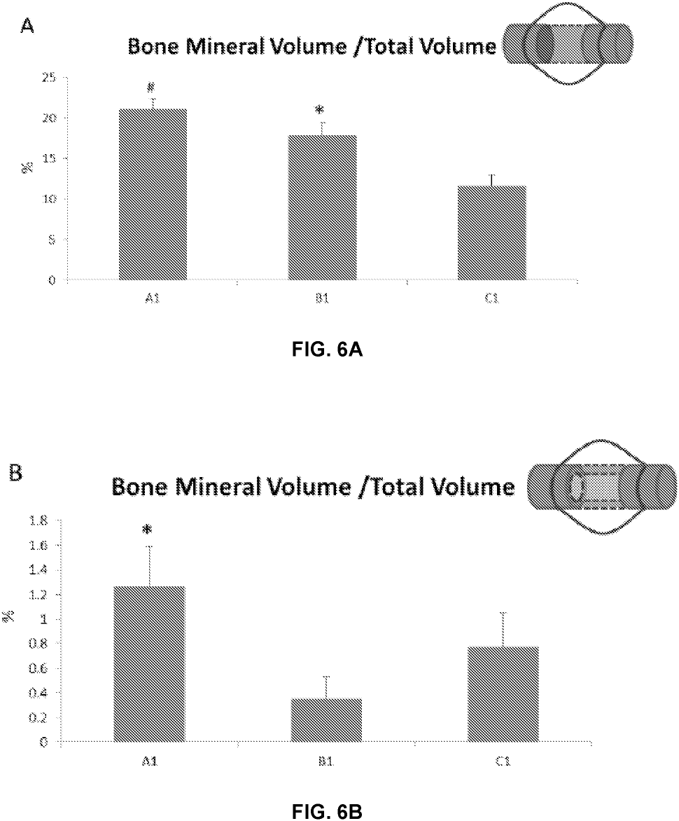

The mechanical testing of femoral strength in each fracture specimen required that the contribution of the rat-fix plate be removed without disturbing the callus. Typically this would involve removing the rat-fix plate altogether. However, in numerous samples, in particular in Group A, the extent of callus enveloped the plate making removal impractical. Thus, the compromise was to cut the plate exactly at the mid-point such that it no longer contributed to femoral support. The cut was done in such a way as to only cause minimal damage to the callus. The downward motion of the upper anvil thus tests the resistance, and thus the strength, of the callus. Group A exhibited markedly increased ULF at 4 and 8 week post-surgery when compared to Groups B and C (FIG. 8). At 8 weeks post-surgery, Group A exhibited 2.5-fold increase in ULF when compared to Group B at the same time point (P<0.001). Group B demonstrated increased ULF when compared to Group C and both time points. At 8 weeks post-surgery, in Group B, a trend for increased ULF was observed when compared to 4 weeks post-surgery (P=0.059).

### Histomorphometry of Treated Femur

At ten days prior to end-point, all animals were administered calcein green (25 mg/kg of 10 mg/mL), with the exception of Rat # 6, 8, 9, 10, 18, 20, 23, 30 and 50, which were inadvertently administered 12.5 mg/Kg of 10 mg/mL solution. All rats received 100 mg/Kg of xylenol orange (100 mg/mL solution) at three days prior to end-point. All administrations were intraperitoneal.

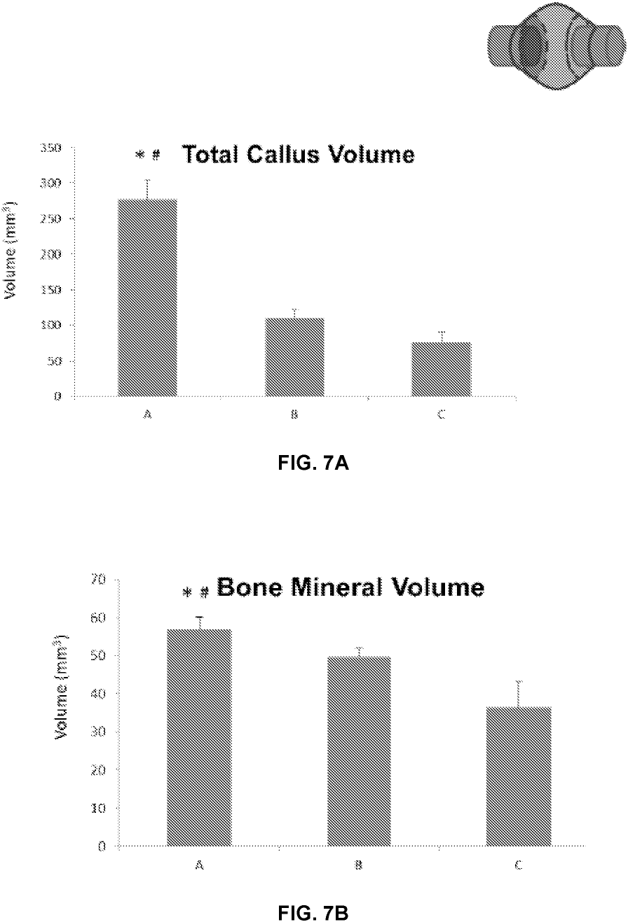

At 4 weeks post-surgery, Group A exhibits extensive evidence of mineralization akin to mature trabecular bone (FIG. 9A). Significant remodelling of bone was observed by the presence of osteoclasts (FIG. 9B, white arrows), and the presence of osteocytes in Group A specimens (FIG. 9B, yellow arrows) which is indicative of mature mineralized bone. Osteoblastic activity was observed in the form of double-fluorochrome labelled mineral accretion. The osteoblastic bone formation was frequently observed as ‘lines’ colored green and red which are in association with each other (FIG. 9B, blue arrows). This represented the formation of lamella (mature) bone, rather than the ad-hoc mineralization of woven bone that occurs in early callus formation, a common observation in Group B. Furthermore, the presence of red-blood cells, and adipocytes indicated that an angiogenesis has occurred and suggested good infiltration of cells to most regions of the callus in Group A specimens.

In Group B, while bone mineral was observed, there were extensive regions of fibrous and cartilage tissue which was unmineralized within the callus region (FIG. 9A). Where new bone mineral occurs, double labels were observed indicating bone formation. However, these double labels were frequently disorganized and often occured as single labels suggesting that woven bone formation (i.e. initial bone callus formation) was continuing to be formed at this time point (FIG. 9B). While not quantified, it appeared that osteoclasts predominantly existed in regions between the unmineralized and mineralized portions of the callus which suggested an earlier stage of callus development than what was observed for Group A animals.

In Group C, there was no evidence of mineralization, bone formation or bone remodelling. Indeed, there was no evidence of cartilage formation. A fibrous-like material with foreign mineralized spicules existed within the fracture site. There were some instances where osteoclasts as present on the surface of the foreign material, suggesting that this material could be resorbed (FIG. 9B).

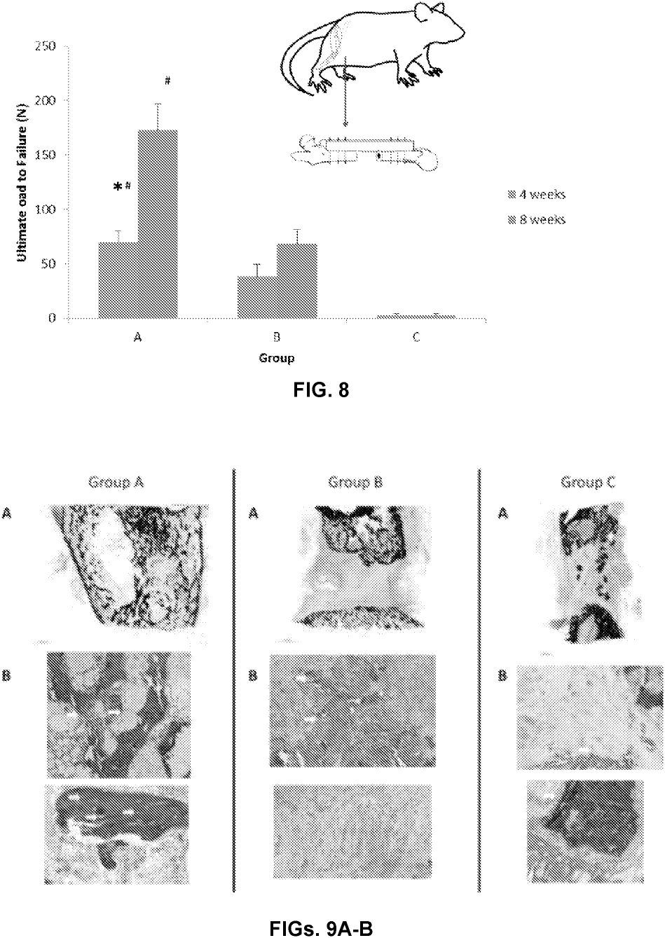

At 8 weeks post-surgery, the same pattern of activity between the groups was continued (FIG. 10A and FIG. 10B). Of note, however, in Group A, there appeared to be greater distribution of mature bone across all sections, consistent with µCT analyses. Also, a degree of intra-trabecular, or intra-cortical labelling of mineral (FIG. 10B, orange arrows) suggested a further maturing of bone consistent with late-stage bone healing. Also of note, Group B specimens appeared to exhibit all stages of callus formation and remodelling. That is, there was evidence of lack of periosteal bridging at times, the presence of cartilage, woven bone, lamellae bone, and evidence of remodelling as well. In Group C, the persistent lack of healing suggested that this group contained a treatment which was most comparable to a critical-sized defect model without any scaffold of healing agents.

In summary, sixty-one animals were successfully treated with one of three test items following surgical creation of a critical size defect in the right femur. The post operative recovery was unremarkable in the majority of animals. The test items were all placed within the defects, but on X-ray, it could be seen that the compounds containing the β-TCP putty were spread out of the defect area and into the surrounding tissue. As the collagen sponge was not visible on X-ray, it was not possible to judge if the sponge had moved out of the defect area.

Most animals recovered from surgery well and in most cases were moving as per usual within 24 hours of surgery. A number of the animals were larger than the recommended weight for the plates, but the femurs were not so large that the screws of the internal fixator system were not going through the whole bone, so there should not have been loss of stability while new bone was forming at the site. Two animals had evidence of displaced bones on the two week radiographs, these animals were not heavier than others. One animal was in the rhBMP +ACS group, the other was in the β-TCP putty group. Another animal (Group C) had a broken screw and displaced bone at the six-week post-operative time point. There was no obvious reason why this had occurred.

There were no differences between any of the groups in any clinical pathology parameters or histological analyses of organs and tissues.

Radiographic and histological examination of the treated femurs showed that the Group A animals (tBMP-2+β-TCP putty) had the most evidence of formation of mature bone in the defect site compared to the other two groups. This was shown by bridging of the periosteal callus, increased mineralization, presence of osteocytes, active remodelling of the bone (as shown by the presence of osteoclasts) and evidence of angiogenesis (shown by the presence of red blood cells and adipocytes). Whilst some evidence of mineralization of callus is present in the Group B (rhBMP+ACS) samples, this appeared to be less developed than for Group A. Group C (β-TCP putty) showed no evidence of mineralization, suggesting that having a scaffold alone could not induce new bone growth within eight weeks of application into a critical-sized defect.

Group A animals also had significantly stronger bones than either Group B or Group C animals as evidenced by ultimate load to failure tests. This was evident at both four and eight weeks post-treatment.

The differences in callus formation in the process of healing are distinct between all three groups, clearly indicating that there were unambiguous differences in the treatments of fracture in each group.

In Group C, a frank lack of periosteal bridging and minimal bony callus formation at both 4 and 8 weeks was associated with a lack of cellular activity within the fracture. These observations were consistent with the near complete absence of mechanical integrity at either time point.

In Group B, the mineralized callus was more extensive than the levels in Group C both 4 and 8 weeks post-surgery. This was evident in the radiographic and histomorphometric analyses. Furthermore, while periosteal bridging was observed only in some Group B specimens at 8 week post-surgery, there was a clear and overt difference in the histological assessment at both time points with regards to bone formation and cellular activity. Unlike in Group C, the presence of bone formation, frequently in the form of woven bone and occasionally as lamellae bone, was associated with increase in mechanical strength when compared to Group C. Interestingly, neither radiographic nor mechanical strength measures were significantly improved at 8 weeks when compared to 4 weeks post-surgery. This suggested that the majority of activity in callus formation and structural integrity occurred in the first 4 weeks.

Group A exhibited the most advanced healing when compared to the other groups. At 4 weeks post-surgery radiographic evidence of periosteal bridging was a measure that no samples achieved in Group C and often was not observed in Group B, even at 8 weeks post-surgery. Interestingly, while radiographic scores at 8 weeks post-surgery were comparable to 4 week scores, the mechanical strength of the callus was more than double the levels at 4 weeks post-surgery and at least 3-fold greater than levels in Group B, suggesting that considerable bone mineralization occurred within the callus region in the second 4 week period. This observation was consistent with high levels of lamellae bone formation in Group A at 4 and 8 weeks post-surgery, which was distinct from the weaker woven bone formation that occurred in Group B, even at 8 weeks post-surgery. It is important to note that callus volume at 8 weeks of age was lower than levels at 4 weeks post-surgery, suggesting that callus volume was being remodelled into a more compact callus at the site of fracture. In Group A, this was consistent with fewer observations of extraneous mineralized callus distal to the fracture site at 8 weeks post-surgery. With regards to the histological assessment of Group A specimens, observations of osteoclastic none resorption adjacent to regions of bone undergoing bone formation is entirely typical of bone undergoing remodelling which is known to be done to provide a stronger and more efficient structure for skeletal integrity. The additional observations of the presence of osteocytes as well as other cellular structures such as vascular structures and adipocytes clearly makes the bone often indistinguishable from endogenous trabecular micro-anatomy.

In conclusion, the bone specimens form the tBMP-2 +β-TCP putty group clearly demonstrated vastly improved bone healing at 4- and 8-weeks post-surgery when compared to samples from animals in the rhBMP +ACS or the β-TCP putty alone treatment groups.

### Example 3. Overview of Chimeric P-TCP Polypeptides

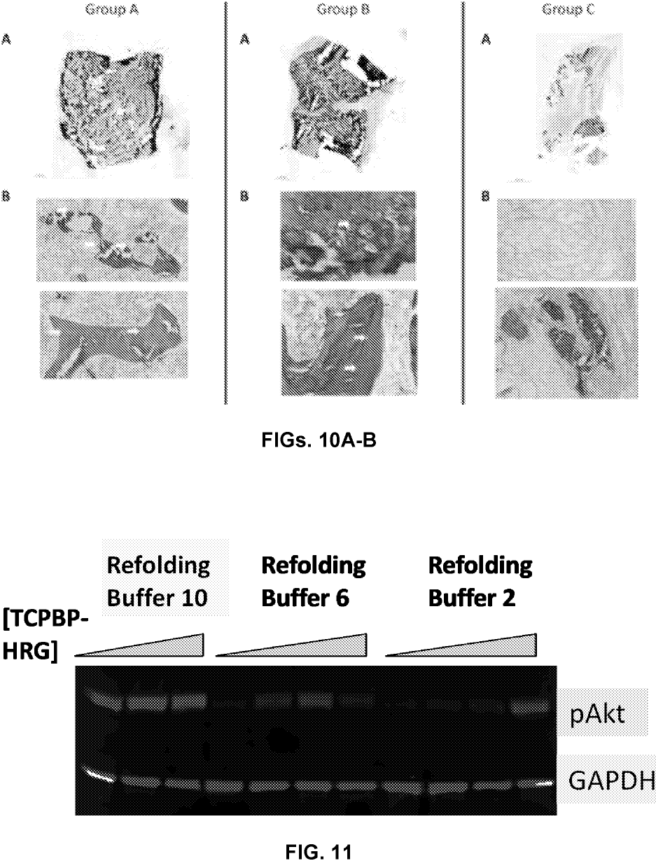

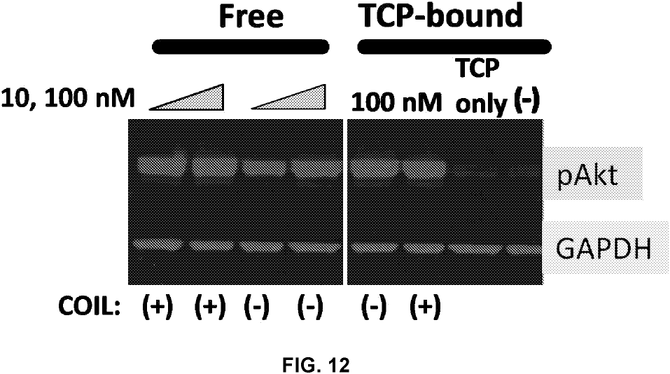

The present inventors found that chimeric polypeptides maintained activity of the tethered protein. The chimeric polypeptide (10x β-TCP-histidine rich glycoprotein HRG) refolded at 22° C. in buffer 10, and following incubation of the chimeric polypeptide with MCF-7 breast cancer cells, Akt activity was detected (FIG. 11). This result indicated that the chimeric polypeptide stimulated signalling activity in the target cell and was able to cause Akt phosphorylation. Next, the Akt activity was determined in 10x β-TCP-HRG bound to a β-TCP peptide (FIG. 12). The β-TCP peptide on its own was unable to cause Akt phosphorylation in MCF-7 cells, however, incubation of MCF-7 cells with β-TCP peptide bound to 10 × β-TCP-HRG led to Akt phosphorylation. Interestingly, the extent of Akt phosphorylation was not affected whether the chimeric polypeptide was bound to a β-TCP peptide or not.

### Example 4. Binding Affinity of tBMP2 to Various Substrate Materials

Binding assays were performed to quantify the affinity of tBMP2 protein to various substrates, including commercially available bone graft materials, ceramic powders, 3D printable scaffold materials, and a plasma-sprayed hydroxyapatite coating. First, 10 mg of each material was washed with deionized water followed by acetate buffer. Then either a 150 µg or 250 µg load of tBMP2 protein was applied to each material and allowed to bind under light agitation for 90 -120 minutes. The flow through was aspirated (removing the tBMP2 which did not bind to the substrate material), and a wash was conducted with acetate buffer. A subsequent overnight elution was done by immersing the substrate material in a sodium phosphate buffer (100 mM NaPhos, 4 M Urea, 1 M NaCl, 10% 1,6 Hexanediol, pH=8) to remove the bound protein from the substrate for measurement.

The bound tBMP2 was quantified by either 1) running non-reducing SDS Page gel electrophoresis and comparing the eluted tBMP2 band to the tBMP2 load band with Image J Gel Analysis tool, or 2) conducting a Bradford assay (with a BSA standard curve) on the eluted tBMP2 and tBMP2 load. Dividing the eluted tBMP2 quantity by the original tBMP2 load quantity and multiplying by 100 gave the % of tBMP2 load that was strongly tethered to the substrate after the binding incubation step.

Table 6 summarizes the materials that were evaluated as well as the % of tBMP2 load which was effectively tethered to the substrate (% Bound).

TABLE 6

Summary of Binding Activity

| Product | Description | Manufacturer | Load tBMP2 Applied to Material | Method of measurement | % of tBMP2 load bound to material |
|---|---|---|---|---|---|
| Mastergraft Strip | Biphasic TCP (85% β-Tricalcium Phosphate: 15% Hydroxyapatite )/Type I bovine collagen composite | Medtronic | 15 ug tBMP2/mg material | Bradford Assay | 69% |
| Vitoss Foam Pack | β-Tricalcium Phosphate/Type I bovine collagen composite | Stryker | 15 ug tBMP2/mg material | Bradford Assay | 82% |
| chronOS Strip | β-Tricalcium Phosphate/poly (lactide co-ε-caprolactone) composite | DePuy Synthes | 15 ug tBMP2/mg material | Image J Gel Analysis Tool | 75% |
| Vitoss Micromors els | β-Tricalcium Phosphate granules (1 mm-2 mm) | Stryker | 15 ug tBMP2/mg material | Image J Gel Analysis Tool | 74% |
| LifeInk 500 | Calcium Phosphate Cement 3D printable ink | Advanced Biomatrix | 15 ug tBMP2/mg material | Image J Gel Analysis Tool | 87% |
| Hyperelasti c Bone | Hydroxapatite/ Poly(lactic-co-glycolic acid) 3D printable ink | Dimension Inx | 15 ug tBMP2/mg material | Image J Gel Analysis Tool | 58% |
| Bioactive Glass | Combeite 45S5 Bioactive Glass powder | Stryker | 25 ug tBMP2/mg material | Image J Gel Analysis Tool | 49% |
| Silicon Nitride Powder | Si₃ N₄ powder, 99% purity | Chemsavers | 25 ug tBMP2/mg material | Image J Gel Analysis Tool | 54% |
| β-TCP Powder | β-Tricalcium Phosphate powder, 3-5 µm particle size | CaP Biomaterials | 25 ug tBMP2/mg material | Image J Gel Analysis Tool | 79% |
| β-TCP Spray Dried Powder | β-Tricalcium Phosphate spray dried powder, <38 µm particle size | CaP Biomaterials | 25 ug tBMP2/mg material | Image J Gel Analysis Tool | 78% |
| Hydroxyap atite Powder | Hydroxyapatite powder, 3-5 µm particle size | CaP Biomaterials | 25 ug tBMP2/mg material | Image J Gel Analysis Tool | 57% |
| HA-coated Bone Screw | Hydroxyapatite plasma spray-coated stainless steel bone screw (6 mm dia x 5 mm long piece cut from screw) | Citieffe | 25 ug tBMP2/mg material | Bradford Assay | 44% |
| β-TCP Granules | β-Tricalcium Phosphate granules, 250-1000 µm particle size | CaP Biomaterials | 15 ug tBMP2/mg material | Bradford Assay | 70% |
| Hydroxyap atite Granules | Hydroxyapatite granules, 250-1000 µm particle size | CaP Biomaterials | 15 ug tBMP2/mg material | Bradford Assay | 52% |
| ReBOSSIS | Cotton-like bone void filler. Main ingredients are β-Tricalcium Phosphate, bioabsorbable polymer, and SiV (silicon-containing calcium carbonate) | Orthorebirth | 15 ug tBMP2/mg material | Bradford Assay | 83% |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising:
(a) a chimeric polypeptide comprising (i) one or more β-tricalcium phosphate (β-TCP)-binding sequence(s) and (ii) a mammalian growth factor; and
(b) a graft material,
wherein the chimeric polypeptide binds specifically to the graft material.

2. The composition of claim 1, wherein the at least one of the one or more (β-TCP-binding sequence(s) is selected from the group consisting of: VIGESTHHRPWS (SEQ ID NO: 2), LIADSTHHSPWT (SEQ ID NO: 3), ILAESTHHKPWT (SEQ ID NO: 4), ILAETTHHRPWS (SEQ ID NO: 5), IIGESSHHKPFT (SEQ ID NO: 6), GLGDTTHHRPWG (SEQ ID NO: 7), VLGDTTHHKPWT (SEQ ID NO: 8), IVADSTHHRPWT (SEQ ID NO: 9); STADTSHHRPS (SEQ ID NO: 10), TSGGESTHHRPS (SEQ ID NO: 11), TSGGESSHHKPS (SEQ ID NO: 12), TGSGDSSHHRPS (SEQ ID NO: 13), GSSGESTHHKPST (SEQ ID NO: 14), VGADSTHHRPVT (SEQ ID NO: 15), GAADTTHHRPVT (SEQ ID NO: 16), AGADTTHHRPVT (SEQ ID NO: 17), GGADTTHHRPAT (SEQ ID NO: 18), and GGADTTHHRPGT (SEQ ID NO: 19).

3. The composition of claim 1 or 2, wherein the chimeric polypeptide comprises two or more β-TCP-binding sequences.

4. The composition of claim 3, wherein each neighboring pair of the two or more (β-TCP binding sequences directly abut each other.

5. The composition of claim 3, wherein each neighboring pair of the two or more (β-TCP binding sequences are separated by a linker sequence.

6. The composition of claim 3, wherein the chimeric polypeptide comprises at least two different β-TCP-binding sequences.

7. The composition of claim 3, wherein the chimeric polypeptide comprises two or more copies of the same β-TCP-binding sequence.

8. The composition of claim 6, wherein at least one of the two or more β-TCP-binding sequences is LLADTTHHRPWT (SEQ ID NO: 1) or GQVLPTTTPSSP (SEQ ID NO: 19).

9. The composition of claim 6, wherein the at least two different β-TCP-binding sequences comprise LLADTTHHRPWT (SEQ ID NO: 1) and VIGESTHHRPWS (SEQ ID NO: 2).

10. The composition of claim 9, wherein the chimeric polypeptide comprises the sequence of LLADTTHHRPWTVIGESTHHRPWS (SEQ ID NO: 20).

11. The composition of claim 6, wherein the at least two different β-TCP-binding sequences comprise LLADTTHHRPWT (SEQ ID NO: 1), VIGESTHHRPWS (SEQ ID NO: 2), and IIGESSHHKPFT (SEQ ID NO: 6).

12. The composition of claim 11, wherein the chimeric polypeptide comprises the sequence of LLADTTHHRPWTVIGESTHHRPWSIIGESSHHKPFT (SEQ ID NO: 21).

13. The composition of claim 6, wherein the at least two different β-TCP-binding sequences comprise LLADTTHHRPWT (SEQ ID NO: 1), VIGESTHHRPWS (SEQ ID NO: 2), IIGESSHHKPFT (SEQ ID NO: 6), GLGDTTHHRPWG (SEQ ID NO: 7), and ILAESTHHKPWT (SEQ ID NO: 4).

14. The composition of claim 13, wherein the chimeric polypeptide comprises the sequence of LLADTTHHRPWTVIGESTHHRPWSIIGESSHHKPFTGLGDTTHHRPWGILAESTHHKP WT (SEQ ID NO: 22).

15. The composition of claim 6, wherein the at least two β-TCP-binding sequences comprise LLADTTHHRPWT (SEQ ID NO: 1) and ILAESTHHKPWT (SEQ ID NO: 4).

16. The composition of claim 15, wherein the chimeric polypeptide comprises at least one copy of the sequence LLADTTHHRPWTILAESTHHKPWT (SEQ ID NO: 23).

17. The composition of claim 16, wherein the chimeric polypeptide comprises the sequence of LLADTTHHRPWTILAESTHHKPWTLLADTTHHRPWTILAESTHHKPWTLLADTTHH RPWT (SEQ ID NO: 24).

18. The composition of claim 6, wherein the at least two β-TCP-binding sequences comprise LLADTTHHRPWT (SEQ ID NO: 1) and GLGDTTHHRPWG (SEQ ID NO: 7).

19. The composition of claim 18, wherein the chimeric polypeptide comprises at least one copy of the sequence of LLADTTHHRPWTGLGDTTHHRPWG (SEQ ID NO: 25).

20. The composition of claim 19, wherein the chimeric polypeptide comprises the sequence of LLADTTHHRPWTGLGDTTHHRPWGLLADTTHHRPWT (SEQ ID NO: 26).

21. The composition of claim 19, wherein the chimeric polypeptide comprises the sequence of LLADTTHHRPWTGLGDTTHHRPWGLLADTTHHRPWTGLGDTTHHRPWGLLADTTH HRPWT (SEQ ID NO: 27).

22. The composition of claim 19, wherein the chimeric polypeptide comprises the sequence of LLADTTHHRPWTGLGDTTHHRPWGLLADTTHHRPWTGLGDTTHHRPWGLLADTTH HRPWTGLGDTTHHRPWGLLADTTHHRPWT (SEQ ID NO: 28).

23. The composition of claim 6, wherein the at least two β-TCP-binding sequences comprise STADTSHHRPS (SEQ ID NO: 10), TSGGESTHHRPS (SEQ ID NO: 11), TSGGESSHHKPS (SEQ ID NO: 12), TGSGDSSHHRPS (SEQ ID NO: 13), and GSSGESTHHKPST (SEQ ID NO: 14).

24. The composition of claim 23, wherein the chimeric polypeptide comprises the sequence of STADTSHHRPSTSGGESTHHRPSTSGGESSHHKPSTGSGDSSHHRPSGSSGESTHHKPS T (SEQ ID NO: 29).

25. The composition of claim 6, wherein the at least two β-TCP-binding sequences comprise VGADSTHHRPVT (SEQ ID NO: 15), GAADTTHHRPVT SEQ ID NO: 16), AGADTTHHRPVT (SEQ ID NO: 17), GGADTTHHRPAT (SEQ ID NO: 18) and GGADTTHHRPGT (SEQ ID NO: 19).

26. The composition of claim 25, wherein the chimeric polypeptide comprises the sequence of VGADSTHHRPVTGAADTTHHRPVTAGADTTHHRPVTGGADTTHHRPATGGADTTH HRPGT (SEQ ID NO: 30).

27. The composition of claim 6, wherein the at least two different β-TCP-binding sequences comprise STADTSHHRPS (SEQ ID NO: 10), LLADTTHHRPWT (SEQ ID NO: 1), TSGGESTHHRPS (SEQ ID NO: 11), VGADSTHHRPVT (SEQ ID NO: 15), TSGGESSHHKPS (SEQ ID NO: 12), GAADTTHHRPVT (SEQ ID NO: 16), TGSGDSSHHRPS (SEQ ID NO: 13), GSSGESTHHKPS (SEQ ID NO: 14), and TGGADTTHHRPAT (SEQ ID NO: 18).

28. The composition of claim 27, wherein the chimeric polypeptide comprises the sequence of STADTSHHRPSLLADTTHHRPWTTSGGESTHHRPSVGADSTHHRPVTTSGGESSHHK PSGAADTTHHRPVTTGSGDSSHHRPSGSSGESTHHKPSTGGADTTHHRPAT (SEQ ID NO: 31).

29. The composition of any one of claims 1-28, wherein the mammalian growth factor is selected from the group consisting of: epidermal growth factor (EGF), platelet derived growth factor (PDGF), insulin like growth factor (IGF-1), fibroblast growth factor (FGF), fibroblast growth factor 2 (FGF2), fibroblast growth factor 18 (FGF18), transforming growth factor alpha (TGF-α), transforming growth factor beta (TGF-β), transforming growth factor beta 1 (TGF-β1), transforming growth factor beta 3 (TGF-β3), osteogenic protein 1 (OP-1), osteogenic protein 2 (OP-2), osteogenic protein 3 (OP-3), bone morphogenetic protein 2 (BMP-2), bone morphogenetic protein 3 (BMP-3), bone morphogenetic protein 4 (BMP-4), bone morphogenetic protein 5 (BMP-5), bone morphogenetic protein 6 (BMP-6), bone morphogenetic protein 7 (BMP-7), bone morphogenetic protein (BMP-9), bone morphogenetic protein 10 (BMP-10), bone morphogenetic protein 11 (BMP-11), bone morphogenetic protein 12 (BMP-12) bone morphogenetic protein 13 (BMP-13), bone morphogenetic protein 15 (BMP-15), dentin phosphoprotein (DPP), vegetal related growth factor (Vgr), growth differentiation factor 1 (GDF-1), growth differentiation factor 3 (GDF-3), growth differentiation factor 5 (GDF-5), growth differentiation factor 6 (GDF-6), growth differentiation factor 7 (GDF-7), growth differentiation factor 8 (GDF8), growth differentiation factor 11 (GDF11), growth differentiation factor 15 (GDF15), vascular endothelial growth factor (VEGF), hyaluronic acid binding protein (HABP), collagen binding protein (CBP), fibroblast growth factor 18 (FGF-18), keratinocyte growth factor (KGF), tumor necrosis factor alpha (TNFα), tumor necrosis factor (TNF)- related apoptosis inducing ligand (TRAIL), wnt family member 1 (WNT1), wnt family member 2 (WNT2), wnt family member 2B (WNT2B), wnt family member 3 (WNT3), wnt family member 3A (WNT3A), wnt family member 4 (WNT4), wnt family member 5A (WNT5A), wnt family member 5B (WNT5B), wnt family member 6 (WNT6), wnt family member 7A (WNT7A), wnt family member 7B (WNT7B), wnt family member 8A (WNT8A), wnt family member 8B (WNT8B), wnt family member 9A (WNT9A), wnt family member 9B (WNT9B), wnt family member 10A (WNT10A), wnt family member 10B (WNT10B), wnt family member 11 (WNT11), and wnt family member 16 (WNT16).

30. The composition of claim 29, wherein the mammalian growth factor is BMP-2.

31. The composition of claim 30, wherein the BMP-2 comprises an amino acid sequence of MVAGTRCLLALLLPQVLLGGAAGLVPELGRRKFAAASSGRPSSQPSDEVLSEFELRL LSMFGLKQRPTPSRDAVVPPYMLDLYRRHSGQPGSPAPDHRLERAASRANTVRSFH HEESLEELPETSGKTTRRFFFNLSSIPTEEFITSAELQVFREQMQDALGNNSSFHHRINI YEIIKPATANSKFPVTRLLDTRLVNQNASRWESFDVTPAVMRWTAQGHANHGFVVE VAHLEEKQGVSKRHVRISRSLHQDEHSWSQIRPLLVTFGHDGKGHPLHKREKRQAK HKQRKRLKSSCKRHPLYVDFSDVGWNDWIVAPPGYHAFYCHGECPFPLADHLNSTN HAIVQTLVNSVNSKIPKACCVPTELSAISMLYLDENEKVVLKNYQDMVVEGCGCR (SEQ ID NO: 32).

32. The composition of claim 31, wherein the BMP-2 consists of the amino acid of SEQ ID NO: 32.

33. The composition of any one of claims 1-32, wherein the chimeric polypeptide further comprises a linker sequence between a neighboring pair of a β-TCP-binding sequence and the growth factor.

34. The composition of claim 33, wherein the linker sequence comprises TGGSGEGGTGASTGGSAGTGGSGGTTSGEAGGSSGAG (SEQ ID NO: 33).

35. The composition of claim 34, wherein the linker sequence consists of SEQ ID NO: 33.

36. The composition of claim 33, wherein the linker sequence comprises GAGTG (SEQ ID NO: 34).

37. The composition of claim 36, wherein the linker sequence consists of SEQ ID NO: 34.

38. The composition of any one of claims 1-37, wherein the chimeric polypeptide binds to β-TCP with a K_{D} of about 1 picomolar to about 100 micromolar.

39. The composition of any one of claims 1-38, wherein the graft material is a bone graft material.

40. The composition of claim 39, wherein the bone graft material is a bone allograft.

41. The composition of claim 39, wherein the bone graft material is an autologous bone graft.

42. The composition of claim 39, wherein the bone graft material is a demineralized bone matrix.

43. The composition of claim 39, wherein the bone graft material is a comminuted bone material.

44. The composition of claim 43, wherein the comminuted bone graft material comprises pulverized bone.

45. The composition of claim 39, wherein the bone graft material comprises at least a portion of a fresh bone, freeze-dried bone, frozen bone, cadaveric bone, or a combination thereof.

46. The composition of claim 39, wherein the bone graft material is a cortical bone graft material.

47. The composition of claim 39, wherein the bone graft material is a cancellous bone graft material.

48. The composition of claim 39, wherein the bone graft material is shaped as a disc, a wafer, a wedge, or a custom shape designed to fill a void in a bone.

49. The composition of any one of claims 1-38, wherein the composition is a powder, a putty, a paste, a coating, an aerosol, a scaffold, a substrate, a liquid, or any combinations thereof.

50. The composition of any one of claims 1-38, wherein the graft material is a soft tissue graft.

51. The composition of any one of claims 1-38, wherein the graft material is a synthetic graft.

52. The composition of any one of claims 1-38, wherein the graft material is a hybrid graft comprising one or more synthetic materials and one or more naturally-derived materials.

53. The composition of any one of claims 1-38, wherein the graft material is a xenograft.

54. The composition of claim 1, wherein the graft material comprises hydroxyapatite.

55. A method of promoting bone or cartilage formation in a subject in need thereof, the method comprising administering to the subject a therapeutically effective amount of any of the compositions of claims 1-53.

56. A method of replacing and/or repairing bone or cartilage in a subject in need thereof, the method comprising administering to the subject a therapeutically effective amount of any of the compositions of claims 1-53.

57. A method of treating a bone fracture or bone loss in a subject in need thereof, the method comprising administering to the subject a therapeutically effective amount of any of the compositions of claims 1-53.

58. The method of any one of claims 54-56, wherein the subject has a bone fracture.

59. The method of any one of claims 54-56, wherein the subject has a bone defect.

60. The method of claim 54 or 55, wherein the subject has a cartilage tear or cartilage defect.

61. The method of claim 54 or 55, wherein the subject has cartilage loss.

62. A kit comprising a composition of any one of claims 1-53.
